SEQUENCE LISTING (6 sequences)

SEQ ID NO: 1 - DNA, 21 bp, synthetic construct
  source 1..21: mol_type = other DNA
tacccctttaatcccaaaccc

SEQ ID NO: 2 - DNA, 41 bp, synthetic construct
  source 1..41: mol_type = other DNA
atctaactgctgcgccgccgggaaaatactgtacggttaga

SEQ ID NO: 3 - DNA, 51 bp, synthetic construct
  source 1..51: mol_type = other DNA
acttattcaattcctgtgggaaggctatagaggggccagtctatgaataag

SEQ ID NO: 4 - DNA, 53 bp, synthetic construct
  source 1..53: mol_type = other DNA
tgactgatttacggctatgggtggtctggttgggattggccccgggagctggc

SEQ ID NO: 5 - DNA, 11 bp, synthetic construct
  source 1..11: mol_type = other DNA
aaaaaaaaaaa

SEQ ID NO: 6 - DNA, 13 bp, synthetic construct
  source 1..13: mol_type = other DNA
ttttttttttttt

(19) United States
(12) Patent Application Publication
(10) Pub. No.: US 2023/0304020 A1
(43) Pub. Date: Sep. 28, 2023

(54) LENTIVIRAL-FREE CYTOSOLIC DELIVERY OF PAYLOADS VIA APTAMERS

(71) Applicant: 10x Genomics, Inc., Pleasanton, CA (US)

(72) Inventor: Wyatt James MCDONNELL, Pleasanton, CA (US)

(21) Appl. No.: 18/061,711

(22) Filed: Dec. 5, 2022

Related U.S. Application Data
(60) Provisional application No. 63/285,962, filed on Dec. 3, 2021.

(51) Int. Cl. C12N 15/115; C12N 9/22; C12Q 1/6818

(52) U.S. Cl. CPC C12N 15/115; C12N 9/22; C12Q 1/6818; C12N 2310/16; C12N 2310/20

(57) ABSTRACT

Provided herein are compositions, systems, methods, and kits for internalizing a payload into a cell via aptamer-mediated deliver. Payloads can comprise molecules capable of hybridizing to nucleic acid sequences directly or through intermediaries (e.g. payload handles). More specifically, payloads can comprise gene editing machinery. A non-limiting example of gene editing machinery can comprise Clustered Regularly Interspaced Short Palindromic Repeats (“CRISPR”) RNP complexes or the subcomponents to those complexes (e.g. gRNA molecules and endonucleases).

### CROSS-REFERENCE TO RELATED APPLICATIONS

This application claims benefit of priority to U.S. Provisional Application Ser. No. 63/285,962, filed Dec. 3, 2021, the entire contents of which are incorporated herein by reference for all purposes.

### SEQUENCE LISTING

The instant application contains a Sequence Listing which has been submitted electronically in xml format and is hereby incorporated by reference in its entirety. Said xml file, created on May 20, 2023, is named 057862-614001US.xml and is 6,388 bytes in size.

### FIELD OF THE INVENTION

This description is generally directed towards compositions, systems, methods, and kits for internalizing a payload into a cell via aptamer-mediated delivery.

### BACKGROUND

Currently, delivery of gene editing machinery (e.g. CRISPR—RNPs, gRNAs, and endonucleases) typically relies on lentiviral vectors. Unfortunately, there are significant drawbacks to lentiviral-mediated delivery systems. Specifically, drawbacks of these systems include low transfection efficiencies, low cell viability after transfection, and other issues reducing the success rate of introducing material into cells. Accordingly, there is a need for technologies that enable delivery of a variety of different payload types using systems that reduce or eliminate these shortcomings. The present disclosure addresses these and other needs.

### SUMMARY

Aspects of the disclosure comprise a composition for internalizing a payload into a cell according to various embodiments. In various embodiments, the composition can comprise a first aptamer comprising, a 3′ end, a 5′ end, a cell surface binding domain, and a hybridization sequence; and a second aptamer comprising, a 3′ end, a 5′ end, a payload binding domain, and a hybridization sequence. In various embodiments, the hybridization sequence of the first aptamer is configured to hybridize to the hybridization sequence of the second aptamer.

In some embodiments of this aspect of the disclosure, the first aptamer may further comprise a detectable label. In some embodiments, the second aptamer may comprise a detectable label. In some embodiments, the detectable label comprises a fluorescent molecule. In some embodiments, the detectable label comprises a quenching agent configured to interact with the fluorescent molecule and decrease fluorescence. In some embodiments, the detectable label is activated upon internalization of at least one of the aptamers into a cell. In some embodiments, the detectable label is activated upon binding of the second aptamer to a payload. In some embodiments, the detectable label is activated upon binding of the first aptamer to a cell surface molecule.

In some embodiments, at least one of the aptamers comprises an aptamer barcode sequence. In some embodiments, the aptamer barcode sequence is located adjacent to the 3′ end of the at least one aptamer. In some embodiments, the aptamer barcode sequence is located adjacent to the 5′ end of the at least one aptamer. In some embodiments, the aptamer barcode sequence is located within 50 nucleotides of either the 3′ end or the 5′ end of the at least one aptamer. In some embodiments, the aptamer barcode sequence is located at a position on the at least one aptamer that does not comprise a secondary structure.

In some embodiments, the hybridization sequence of the first aptamer comprises a first aptamer barcode sequence and the hybridization sequence of the second aptamer comprises a second aptamer barcode sequence, wherein the first aptamer barcode sequence is a reverse complement of the second aptamer barcode sequence. In some embodiments, hybridization of the first aptamer barcode sequence to the second aptamer barcode sequence is detectable and indicates correct aptamer pairing.

In some embodiments, the aptamer barcode sequences are configured to indicate internalization of the one or more aptamers into a cell.

In some embodiments, the hybridization sequence of the first aptamer is positioned between the cell surface binding domain and the 3′ end of the first aptamer. In some embodiments, the hybridization sequence of the first aptamer is positioned between the cell surface binding domain and the 5′ end of the first aptamer. In some embodiments, the hybridization sequence of the first aptamer is positioned between the cell surface binding domain and the 3′ end of the first aptamer. In some embodiments, the hybridization sequence of the first aptamer is positioned between the cell surface binding domain and the 5′ end of the first aptamer.

In some embodiments, the hybridization sequence of the second aptamer is positioned between the payload binding domain and the 3′ end of the second aptamer. In some embodiments, the hybridization sequence of the second aptamer is positioned between the payload binding domain and the 5′ end of the second aptamer. In some embodiments, each of the hybridization sequences comprises between 12-20 nucleotides.

In some embodiments, the composition further comprises a cell surface molecule bound to the cell surface binding domain. In some embodiments, the cell surface molecule is part of an internalization complex. In some embodiments, the cell surface molecule comprises a receptor tyrosine kinase. In some embodiments, the cell surface molecule comprises a transmembrane protein.

In some embodiments, the payload binding domain further comprises a payload handle. In some embodiments, the payload handle comprises a protein recognition sequence. In some embodiments, the payload handle comprises a biotinylated structure. In some embodiments, payload handle comprises a poly-A tail capture sequence.

Some embodiments further comprise a payload bound to the payload binding domain. In some embodiments, the payload comprises a Cas molecule. In some embodiments, the payload comprises a gRNA molecule. In some embodiments, the payload comprises an RNP complex, wherein the RNP complex comprises a Cas molecule associated with a gRNA molecule. In some embodiments, the payload comprises an mRNA molecule. In some embodiments, the payload comprises an oligonucleotide. In some embodiments, the payload comprises a protein. In some embodiments, the payload comprises an RNAi molecule.

In some embodiments, the cell surface binding domain comprises a secondary structure. In some embodiments, the secondary structure of the cell surface binding domain comprises a stem loop.

In some embodiments, the cell surface binding domain comprises a tertiary structure.

In some embodiments, the payload binding domain comprises a secondary structure. In some embodiments, the secondary structure of the payload binding domain comprises a stem loop.

In some embodiments, the payload binding domain comprises a tertiary structure.

Some embodiments further comprise a bead comprising an oligonucleotide, wherein the oligonucleotide comprises: a nucleic acid barcode molecule comprising a bead specific barcode and a capture sequence, wherein the capture sequence is complementary to at least a portion of at least one of the aptamers. In some embodiments, the oligonucleotide further comprises a unique molecular identifier (UMI).

Aspects of the disclosure comprise a composition for internalizing a plurality of payloads into a cell according to various embodiments. In various embodiments, the composition can comprise a first aptamer comprising, 3′ end, a 5′ end, a cell surface binding domain, and a hybridization sequence; a second aptamer comprising a 3′ end, a 5′ end, a first hybridization sequence, a second hybridization sequence, and a payload binding domain; and a third aptamer comprising a 3′ end, a 5′ end, a payload binding domain, and a hybridization sequence. In various embodiments, the hybridization sequence of the first aptamer is configured to hybridize to the first hybridization sequence of the second aptamer. In various embodiments, the hybridization sequence of the third aptamer is configured to hybridize to the second hybridization sequence of the second aptamer.

In some embodiments in this aspect of the disclosure, the first aptamer further comprises a detectable label. In some embodiments, the second aptamer further comprises a detectable label. In some embodiments, the third aptamer further comprises a detectable label. In any of these embodiments, the detectable label comprises a fluorescent molecule. In some embodiments, the detectable label comprises a quenching agent configured to interact with the fluorescent molecule and decrease fluorescence. In some embodiments, the detectable label is activated upon internalization of at least one of the aptamers into a cell. In some embodiments, the detectable label is activated upon the second aptamer binding a payload. In some embodiments, the detectable label is activated upon the third aptamer binding a payload. In some embodiments, the detectable label is activated upon the first aptamer binding a cell surface molecule.

In some embodiments, at least one of the aptamers comprises an aptamer barcode sequence. In some embodiments, the aptamer barcode sequence is located adjacent to the 3′ end of the at least one aptamer. In some embodiments, the aptamer barcode sequence is located adjacent to the 5′ end of the at least one aptamer. In some embodiments, the aptamer barcode sequence is located within 50 nucleotides of either the 3′ end or the 5′ end of the at least one aptamer. In some embodiments, the aptamer barcode sequence is located at a position on the at least one aptamer that does not comprise a secondary structure.

In some embodiments, the hybridization sequence of the first aptamer comprises a first aptamer barcode sequence and the hybridization sequence of the second aptamer comprises a second aptamer barcode sequence, wherein the first aptamer barcode sequence is a reverse complement of the second aptamer barcode sequence. In some embodiments, hybridization of the first aptamer barcode sequence to the second aptamer barcode sequence is detectable and indicates correct aptamer pairing.

In some embodiments, the second hybridization sequence of the second aptamer comprises a first aptamer barcode sequence and the hybridization sequence of the third aptamer comprises a second aptamer barcode sequence, wherein the first aptamer barcode sequence is a reverse complement to the second aptamer barcode sequence. In some embodiments, hybridization of the first aptamer barcode sequence to the second aptamer barcode sequence is detectable and indicates correct aptamer pairing.

In some embodiments, the aptamer barcode sequences are configured to indicate internalization of the one or more aptamers into a cell.

In some embodiments, the hybridization sequence of the first aptamer is positioned between the cell surface binding domain and the 3′ end of the first aptamer. In some embodiments, the hybridization sequence of the first aptamer is positioned between the cell surface binding domain and the 5′ end of the first aptamer.

In some embodiments, the hybridization sequence of the second aptamer is positioned between the payload binding domain and the 3′ end of the second aptamer. In some embodiments, the hybridization sequence of the second aptamer is positioned between the payload binding domain and the 5′ end of the second aptamer.

In some embodiments, the hybridization sequence of the third aptamer is positioned between the payload binding domain and the 3′ end of the third aptamer. In some embodiments, the hybridization sequence of the third aptamer is positioned between the payload binding domain and the 5′ end of the third aptamer.

In some embodiments, each of the hybridization sequences comprises between 12-20 nucleotides.

Some embodiments further comprise a cell surface molecule bound to the cell surface binding domain. In some embodiments, the cell surface molecule is part of an internalization complex. In some embodiments, the cell surface molecule comprises a receptor tyrosine kinase. In some embodiments, the cell surface molecule comprises a transmembrane protein.

In some embodiments, the at least one of the payload binding domains further comprises a payload handle. In some embodiments, the payload handle comprises a protein recognition sequence. In some embodiments, the payload handle comprises a biotinylated structure. In some embodiments, the payload handle comprises a poly-A tail capture sequence.

Some embodiments further comprise a payload bound to at least one the payload binding domains. In some embodiments, the payload comprises a Cas molecule. In some embodiments, the payload comprises a gRNA molecule. In some embodiments, the payload comprises an RNP complex, wherein the RNP complex comprises a Cas molecule associated with a gRNA molecule. In some embodiments, the payload comprises an mRNA molecule. In some embodiments, the payload comprises an oligonucleotide. In some embodiments, the payload comprises a protein. In some embodiments, payload comprises an RNAi molecule.

In some embodiments, the cell surface binding domain comprises a secondary structure. In some embodiments, the secondary structure of the cell surface binding domain comprises a stem loop.

In some embodiments, the cell surface binding domain comprises a tertiary structure.

In some embodiments, at least one of the payload binding domains comprises a secondary structure. In some embodiments, the secondary structure of the payload binding domain comprises a stem loop.

In some embodiments, at least one of the payload binding domains comprise a tertiary structure.

Some embodiments further comprise a payload bound to each of the payload binding domains. In some embodiments, a payload of the second aptamer is different from a payload of the third aptamer. In some embodiments, a payload of the second aptamer is the same as the payload of the third aptamer.

In some embodiments, a sequence similarity between the hybridization sequence of the first aptamer and the second hybridization sequence of the second aptamer is less than 25%, a sequence similarity between the hybridization sequence of the first aptamer and the hybridization sequence of the third aptamer is less than 25%, and a sequence similarity between the hybridization sequence of the third aptamer and the first hybridization sequence of the second aptamer is less than 25%.

In some embodiments, a sequence similarity between the hybridization sequence of the first aptamer and the second hybridization sequence of the second aptamer is less than 20%, a sequence similarity between the hybridization sequence of the first aptamer and the hybridization sequence of the third aptamer is less than 20%, and a sequence similarity between the hybridization sequence of the third aptamer and the first hybridization sequence of the second aptamer is less than 20%.

In some embodiments, a sequence similarity between the hybridization sequence of the first aptamer and the second hybridization sequence of the second aptamer comprises fewer than 8 consecutive complementary bases, a sequence similarity between the hybridization sequence of the first aptamer and the hybridization sequence of the third aptamer comprises fewer than 8 consecutive complementary bases, and a sequence similarity between the hybridization sequence of the third aptamer and the first hybridization sequence of the second aptamer comprises fewer than 8 consecutive complementary bases.

In some embodiments, the hybridization sequence of the first aptamer and the first hybridization sequence of the second aptamer have a sequence complementarity that ranges from 75% to 100%.

In some embodiments, the hybridization sequence of the third aptamer and the second hybridization sequence of the second aptamer have a sequence complementarity that ranges from 75% to 100%.

Aspects of the disclosure comprise a composition for internalizing a plurality of payloads into a cell according to various embodiments. In various embodiments, the composition can comprise a first aptamer comprising, a 3′ end, a 5′ end, a cell surface binding domain, a first hybridization sequence, and a second hybridization sequence; a second aptamer comprising, a 3′ end, a 5′ end, a hybridization sequence, and a payload binding domain; and a third aptamer comprising, a 3′ end, a 5′ end, a hybridization sequence, and a payload binding domain. In various embodiments, the first hybridization sequence is configured to hybridize to the hybridization sequence of the second aptamer. In various embodiments, the second hybridization sequence is configured to hybridize to the hybridization sequence of the third aptamer.

In some embodiments of this aspect of the disclosure, the first aptamer further comprises a detectable label. In some embodiments, the second aptamer further comprises a detectable label. In some embodiments, third aptamer further comprises a detectable label. In some embodiments, the detectable label comprises a quenching agent configured to interact with the fluorescent molecule and decrease fluorescence. In some embodiments, the detectable label is activated upon internalization of at least one of the aptamers into a cell. In some embodiments, the detectable label is activated upon the second aptamer binding a payload. In some embodiments, the detectable label is activated upon the third aptamer binding a payload. In some embodiments, the detectable label is activated upon the first aptamer binding a cell surface molecule.

In some embodiments, at least one of the aptamers comprises an aptamer barcode sequence. In some embodiments, the aptamer barcode sequence is located adjacent to the 3′ end of the at least one aptamer. In some embodiments, the aptamer barcode sequence is located adjacent to the 5′ end of the at least one aptamer. In some embodiments, the aptamer barcode sequence is located within 50 nucleotides of either the 3′ end or the 5′ end of the at least one aptamer. In some embodiments, the aptamer barcode sequence is located at a position on the at least one aptamer without secondary structure.

In some embodiments, the first hybridization sequence of the first aptamer comprises a first aptamer barcode sequence and the hybridization sequence of the second aptamer comprises a second aptamer barcode sequence, wherein the first aptamer barcode sequence is a reverse complement to the second aptamer barcode sequence. In some embodiments, the first aptamer barcode sequence hybridized to the second aptamer barcode sequence is detectable and indicates correct aptamer pairing.

In some embodiments, the second hybridization sequence of the first aptamer comprises a first aptamer barcode sequence and the hybridization sequence of the third aptamer comprises a second aptamer barcode sequence, wherein the first aptamer barcode sequence is a reverse complement to the second aptamer barcode sequence. In some embodiments, the first aptamer barcode sequence hybridized to the second aptamer barcode sequence is detectable and indicates correct aptamer pairing.

In some embodiments, the aptamer barcode sequences are configured to indicate internalization of the one or more aptamers into a cell.

In some embodiments, the hybridization sequence of the first aptamer is positioned between the cell surface binding domain and the 3′ end of the first aptamer. In some embodiments, the hybridization sequence of the first aptamer is positioned between the cell surface binding domain and the 5′ end of the first aptamer.

In some embodiments, the hybridization sequence of the second aptamer is positioned between the payload binding domain and the 3′ end of the second aptamer. In some embodiments, the hybridization sequence of the second aptamer is positioned between the payload binding domain and the 5′ end of the second aptamer.

In some embodiments, the hybridization sequence of the third aptamer is positioned between the payload binding domain and the 3′ end of the third aptamer. In some embodiments, the hybridization sequence of the third aptamer is positioned between the payload binding domain and the 5′ end of the third aptamer.

In some embodiments, each of the hybridization sequences comprises between 12-20 nucleotides.

Some embodiments further comprise a cell surface molecule bound to the cell surface binding domain. In some embodiments, the cell surface molecule is part of an internalization complex. In some embodiments, the cell surface molecule comprises a receptor tyrosine kinase. In some embodiments, the cell surface molecule comprises a transmembrane protein.

In some embodiments, the at least one of the payload binding domains further comprises a payload handle. In some embodiments, the payload handle comprises a protein recognition sequence. In some embodiments, the payload handle comprises a biotinylated structure. In some embodiments, the payload handle comprises a poly-A tail capture sequence.

Some embodiments further comprise a payload bound to at least one of the payload binding domains. In some embodiments, the payload comprises a Cas molecule. In some embodiments, the payload comprises a gRNA molecule. In some embodiments, the payload comprises an RNP complex, wherein the RNP complex comprises a Cas molecule associated with a gRNA molecule. In some embodiments, the payload comprises an mRNA molecule. In some embodiments, the payload comprises an oligonucleotide. In some embodiments, the payload comprises a protein. In some embodiments, the payload comprises an RNAi molecule.

In some embodiments, the cell surface binding domain comprises a secondary structure. In some embodiments, the cell surface binding domain secondary structure comprises a stem loop.

In some embodiments, the cell surface binding domain comprises a tertiary structure.

In some embodiments, at least one of the payload binding domains comprise a secondary structure. In some embodiments, the payload binding domain secondary structure comprises a stem loop.

In some embodiments, at least one of the payload binding domains comprise a tertiary structure.

Some embodiments further comprise a payload bound to each of the payload binding domains.

In some embodiments, a payload of the second aptamer is different than a payload of the aptamer. In some embodiments, a payload of the second aptamer is the same as the payload of the third aptamer.

In some embodiments, a sequence similarity between the first hybridization sequence of the first aptamer and the hybridization sequence of the third aptamer is less than 25%, a sequence similarity between the second hybridization sequence of the first aptamer and the hybridization sequence of the second aptamer is less than 25%, and a sequence similarity between the hybridization sequence of the second aptamer and the hybridization sequence of the third aptamer is less than 25%.

In some embodiments, a sequence similarity between the first hybridization sequence of the first aptamer and the hybridization sequence of the third aptamer is less than 20%, a sequence similarity between the second hybridization sequence of the first aptamer and the hybridization sequence of the second aptamer is less than 20%, and a sequence similarity between the hybridization sequence of the second aptamer and the hybridization sequence of the third aptamer is less than 20%.

In some embodiments, a sequence similarity between the first hybridization sequence of the first aptamer and the hybridization sequence of the third aptamer comprises fewer than 8 consecutive complementary bases, a sequence similarity between the second hybridization sequence of the first aptamer and the hybridization sequence of the second aptamer comprises fewer than 8 consecutive complementary bases, and a sequence similarity between the hybridization sequence of the second aptamer and the hybridization sequence of the third aptamer comprises fewer than 8 consecutive complementary bases.

In some embodiments, the first hybridization sequence of the first aptamer and the hybridization sequence of the second aptamer have a sequence complementarity that ranges from 75% to 100%.

In some embodiments, the second hybridization sequence of the first aptamer and the hybridization sequence of the third aptamer have a sequence complementarity that ranges from 75% to 100%.

Aspects of the disclosure comprise a system for internalizing one or more payload molecules into a cell according to various embodiments. In various embodiments, the system can comprise a plurality of first internalization complexes, each comprising a first aptamer comprising, a 3′ end, a 5′ end, a cell surface binding domain, and a hybridization sequence, and a second aptamer comprising a 3′ end, a 5′ end, a payload binding domain, and a hybridization sequence. In various embodiments, the hybridization sequence of the first aptamer of one of the plurality of first internalization complexes is configured to hybridize to the hybridization sequence of the second aptamer of the one of the plurality of first internalization complexes.

Some embodiments of these aspects of the disclosure further comprise a plurality of second internalization complexes, each comprising a first aptamer comprising a 3′ end, a 5′ end, a cell surface binding domain, and a hybridization sequence; a second aptamer comprising a 3′ end, a 5′ end, a first hybridization sequence, a second hybridization sequence and a payload binding domain; and a third aptamer comprising a 3′ end, a 5′ end, a payload binding domain and a hybridization sequence, where the hybridization sequence of the first aptamer of one of the plurality of second internalization complexes is configured to hybridize to the first hybridization sequence of the second aptamer of the one of the plurality of second internalization complexes, and the hybridization sequence of the third aptamer of the one of the plurality of second internalization complexes is configured to hybridize to the second hybridization sequence of the second aptamer of the one of the plurality of second internalization complexes.

Some embodiments of these aspects of the disclosure further comprise a plurality of third internalization complexes, each comprising, a first aptamer comprising a 3′ end, a 5′ end, a cell surface binding domain, a first hybridization sequence and a second hybridization sequence; a second aptamer comprising a 3′ end, a 5′ end, a hybridization sequence and a payload binding domain; and a third aptamer comprising a 3′ end, a 5′ end, a hybridization sequence and a payload binding domain, where the first hybridization sequence of the first aptamer of one of the plurality of third internalization complexes is configured to hybridize to the hybridization sequence of the second aptamer of the one of the plurality of third internalization complexes, and the second hybridization sequence of the first aptamer of the one of the plurality of third internalization complexes is configured to hybridize to the hybridization sequence of the third aptamer of the one of the plurality of third internalization complexes.

In some embodiments, at least one of the aptamers of the plurality of first, plurality of second or plurality of third internalization complexes further comprises a detectable label. In some embodiments, the detectable label comprises a fluorescent molecule. In some embodiments, the detectable label comprises a quenching agent configured to interact with the fluorescent molecule and decrease fluorescence. In some embodiments, the detectable label is activated upon internalization of at least one of the aptamers into a cell. In some embodiments, the detectable label is activated upon the second aptamer binding a payload. In some embodiments, the detectable label is activated upon the first aptamer binding a cell surface molecule.

In some embodiments, at least one of the aptamers comprises an aptamer barcode sequence. In some embodiments, the aptamer barcode sequence is located adjacent to the 3′ end of the at least one aptamer. In some embodiments, the aptamer barcode sequence is located adjacent to the 5′ end of the at least one aptamer. In some embodiments, the aptamer barcode sequence is located within 50 nucleotides of either the 3′ end or the 5′ end of the at least one aptamer. In some embodiments, the aptamer barcode sequence is located at a position on the at least one aptamer without secondary structure.

In some embodiments, a hybridization sequence of at least one of the aptamers comprises a first aptamer barcode sequence and specifically targets a hybridization sequence of at least another aptamer comprising a second aptamer barcode sequence, wherein the first aptamer barcode sequence is a reverse complement to the second aptamer barcode sequence. In some embodiments, the first aptamer barcode sequence hybridized to the second aptamer barcode sequence is detectable and indicates correct aptamer pairing.

In some embodiments, the aptamer barcode sequences are configured to indicate internalization of the one or more aptamers into a cell.

In some embodiments, each of the hybridization sequences comprises between 12-20 nucleotides.

Some embodiments further comprise a cell surface molecule bound to the cell surface binding domain. In some embodiments, the cell surface molecule is part of an internalization complex. In some embodiments, the cell surface molecule comprises a receptor tyrosine kinase. In some embodiments, the cell surface molecule comprises a transmembrane protein.

In some embodiments, at least one of the payload binding domains further comprises a payload handle. In some embodiments, the payload handle comprises a protein recognition sequence. In some embodiments, the payload handle comprises a biotinylated structure. In some embodiments, the payload handle comprises a poly-A tail capture sequence.

Some embodiments further comprise a payload bound to the payload binding domain. In some embodiments, the payload comprises a Cas molecule. In some embodiments, the payload comprises a gRNA molecule. In some embodiments, the payload comprises an RNP complex, wherein the RNP complex comprises a Cas molecule associated with a gRNA molecule. In some embodiments, the payload comprises an mRNA molecule. In some embodiments, the payload comprises an oligonucleotide. In some embodiments, the payload comprises a protein. In some embodiments, the payload comprises an RNAi molecule.

In some embodiments, the cell surface binding domain comprises a secondary structure. In some embodiments, the cell surface binding domain secondary structure comprises a stem loop.

In some embodiments, the cell surface binding domain comprises a tertiary structure.

In some embodiments, the payload binding domain comprises a secondary structure. In some embodiments, the payload binding domain secondary structure comprises a stem loop.

In some embodiments, the payload binding domain comprises a tertiary structure.

Some embodiments further comprise a bead comprising an oligonucleotide, where the oligonucleotide comprises a nucleic acid barcode molecule comprising a bead specific barcode; and a capture sequence, wherein the capture sequence is complementary to at least a portion of at least one of the aptamers. In some embodiments, the oligonucleotide further comprises a unique molecular identifier (UMI).

Aspects of the disclosure comprise a system for internalizing a plurality of payloads into a cell according to various embodiments. In various embodiments, the system comprises a plurality of internalization complexes, each comprising, a first aptamer comprising a 3′ end, a 5′ end, a cell surface binding domain, and a hybridization sequence; a second aptamer comprising, a 3′ end, a 5′ end, a first hybridization sequence, a second hybridization sequence, and a payload binding domain; and a third aptamer comprising a 3′ end, a 5′ end, a payload binding domain, and a hybridization sequence. In various embodiments, the hybridization sequence of the first aptamer of one of the plurality of first internalization complexes is configured to hybridize to the first hybridization sequence of the second aptamer of the one of the plurality of first internalization complexes. In various embodiments, the hybridization sequence of the third aptamer of the one of the plurality of first internalization complexes is configured to hybridize to the second hybridization sequence of the second aptamer of the one of the plurality of first internalization complexes.

Some embodiments of these aspects of the disclosure further comprise a plurality of second internalization complexes, where each of the second internalization complexes comprises a first aptamer comprising a 3′ end, a 5′ end, a cell surface binding domain, and a hybridization sequence; and a second aptamer comprising a 3′ end, a 5′ end, a payload binding domain and a hybridization sequence, where the hybridization sequence of the first aptamer of one of the plurality of second internalization complexes is configured to hybridize to the hybridization sequence of the second aptamer of the one of the plurality of second internalization complexes.

Some embodiments of these aspects of the disclosure further comprise a third plurality of internalization complexes, where each of the third internalization complexes comprises a first aptamer comprising a 3′ end, a 5′ end, a cell surface binding domain, a first hybridization sequence and a second hybridization sequence; a second aptamer comprising a 3′ end, a 5′ end, a hybridization sequence and a payload binding domain; and a third aptamer comprising a 3′ end, a 5′ end, a hybridization sequence and a payload binding domain, where the first hybridization sequence of one of the second internalization complexes is configured to hybridize to the hybridization sequence of the second aptamer of the one of the plurality of second internalization complexes, and the second hybridization sequence the third aptamer of the one of the second internalization complexes is configured to hybridize to the hybridization sequence of the third aptamer of the one of the plurality of second internalization complexes.

In some embodiments, at least one of the aptamers of the plurality of first, plurality of second or plurality of third internalization complexes further comprises a detectable label. In some embodiments, the detectable label comprises a fluorescent molecule. In some embodiments, the detectable label comprises a quenching agent configured to interact with the fluorescent molecule and decrease fluorescence. In some embodiments, the detectable label is activated upon internalization of at least one of the aptamers into a cell. In some embodiments, the detectable label is activated upon the second aptamer binding a payload. In some embodiments, the detectable label is activated upon the third aptamer binding a payload. In some embodiments, the detectable label is activated upon the first aptamer binding a cell surface molecule.

In some embodiments, at least one of the aptamers comprises an aptamer barcode sequence. In some embodiments, the aptamer barcode sequence is located adjacent to the 3′ end of the at least one aptamer. In some embodiments, the aptamer barcode sequence is located adjacent to the 5′ end of the at least one aptamer. In some embodiments, the aptamer barcode sequence is located within 50 nucleotides of either the 3′ end or the 5′ end of the at least one aptamer. In some embodiments, the aptamer barcode sequence is located at a position on the at least one aptamer without secondary structure.

In some embodiments, a hybridization sequence of at least one of the aptamers comprises a first aptamer barcode sequence and specifically targets a hybridization sequence of at least another aptamer comprising a second aptamer barcode sequence, wherein the first aptamer barcode sequence is a reverse complement to the second aptamer barcode sequence. In some embodiments, the first aptamer barcode sequence hybridized to the second aptamer barcode sequence is detectable and indicates correct aptamer pairing.

In some embodiments, the aptamer barcode sequences are configured to indicate internalization of the one or more aptamers into a cell.

In some embodiments, each of the hybridization sequences comprises between 12-20 nucleotides.

Some embodiments further comprise a cell surface molecule bound to the cell surface binding domain. In some embodiments, the cell surface molecule is part of an internalization complex. In some embodiments, the cell surface molecule comprises a receptor tyrosine kinase. In some embodiments, the cell surface molecule comprises a transmembrane protein.

In some embodiments, at least one of the payload binding domains further comprises a payload handle. In some embodiments, the payload handle comprises a protein recognition sequence. In some embodiments, the payload handle comprises a biotinylated structure. In some embodiments, the payload handle comprises a poly-A tail capture sequence.

Some embodiments further comprise a payload bound to the payload binding domain. In some embodiments, the payload comprises a Cas molecule. In some embodiments, the payload comprises a gRNA molecule. In some embodiments, the payload comprises an RNP complex, wherein the RNP complex comprises a Cas molecule associated with a gRNA molecule. In some embodiments, the payload comprises an mRNA molecule. In some embodiments, the payload comprises an oligonucleotide. In some embodiments, the payload comprises a protein. In some embodiments, payload comprises an RNAi molecule.

In some embodiments, the cell surface binding domain comprises a secondary structure. In some embodiments, the cell surface binding domain secondary structure comprises a stem loop.

In some embodiments, the cell surface binding domain comprises a tertiary structure.

In some embodiments, the payload binding domain comprises a secondary structure. In some embodiments, the payload binding domain second structure comprises a stem loop.

In some embodiments, the payload binding domain comprises a tertiary structure.

Some embodiments further comprise a bead comprising an oligonucleotide, where the oligonucleotide comprises a nucleic acid barcode molecule comprising a bead specific barcode and a capture sequence, where the capture sequence is complementary to at least a portion of at least one of the aptamers. In some embodiments, the oligonucleotide further comprises a unique molecular identifier (UMI).

Aspects of the disclosure comprise a system for internalizing a plurality of payloads into a cell according to various embodiments. In various embodiments, the system comprises a plurality of first internalization complexes, each comprising a first aptamer comprising, a 3′ end, a 5′ end, a cell surface binding domain, a first hybridization sequence, and a second hybridization sequence; a second aptamer comprising, a 3′ end, a 5′ end, a hybridization sequence, and a payload binding domain; and a third aptamer comprising, a 3′ end, a 5′ end, a hybridization sequence, and a payload binding domain. In various embodiments, the first hybridization sequence of one of the plurality of first internalization complexes is configured to hybridize to the hybridization sequence of the second aptamer of the one of the plurality of first internalization complexes. In various embodiments, the second hybridization sequence of the one of the plurality of first internalization complexes is configured to hybridize to the hybridization sequence of the third aptamer of the one of the plurality of first internalization complexes.

Some embodiments of this aspect of the disclosure further comprise a plurality of second internalization complexes, each comprising a first aptamer comprising a 3′ end, a 5′ end, a cell surface binding domain and a hybridization sequence; and a second aptamer comprising a 3′ end, a 5′ end, a payload binding domain, and a hybridization sequence, wherein the hybridization sequence of the first aptamer of one of the second internalization complexes is configured to hybridize to the hybridization sequence of the second aptamer of the one of the second internalization complexes.

Some embodiments of this aspect of the disclosure further comprise a plurality of third internalization complexes, each comprising a first aptamer comprising a 3′ end, a 5′ end, a cell surface binding domain and a hybridization sequence; a second aptamer comprising a 3′ end, a 5′ end, a first hybridization sequence, a second hybridization sequence, and a payload binding domain; and a third aptamer comprising a 3′ end, a 5′ end, a payload binding domain, and a hybridization sequence, wherein the hybridization sequence of the first aptamer of one of the plurality of third internalization complexes is configured to hybridize to the first hybridization sequence of the second aptamer of the one of the plurality of third internalization complexes, and the hybridization sequence of the third aptamer of the one of the plurality of third internalization complexes is configured to hybridize to the second hybridization sequence of the second aptamer of the one of the plurality of third internalization complexes.

In some embodiments, at least one of the aptamers of the plurality of first, plurality of second or plurality of third internalization complexes further comprises a detectable label. In some embodiments, the detectable label comprises a fluorescent molecule. In some embodiments, the detectable label comprises a quenching agent configured to interact with the fluorescent molecule and decrease fluorescence. In some embodiments, the detectable label is activated upon internalization of at least one of the aptamers into a cell. In some embodiments, the detectable label is activated upon the second aptamer binding a payload. In some embodiments, the detectable label is activated upon the third aptamer binding a payload. In some embodiments, the detectable label is activated upon the first aptamer binding a cell surface molecule.

In some embodiments, at least one of the aptamers comprises an aptamer barcode sequence. In some embodiments, the aptamer barcode sequence is located adjacent to the 3′ end of the at least one aptamer. In some embodiments, the aptamer barcode sequence is located adjacent to the 5′ end of the at least one aptamer. In some embodiments, the aptamer barcode sequence is located within 50 nucleotides of either the 3′ end or the 5′ end of the at least one aptamer. In some embodiments, the aptamer barcode sequence is located at a position on the at least one aptamer without secondary structure.

In some embodiments, a hybridization sequence of at least one of the aptamers comprises a first aptamer barcode sequence and specifically targets a hybridization sequence of at least another aptamer comprising a second aptamer barcode sequence, wherein the first aptamer barcode sequence is a reverse complement to the second aptamer barcode sequence. In some embodiments, the first aptamer barcode sequence hybridized to the second aptamer barcode sequence is detectable indicates correct aptamer pairing.

In some embodiments, the aptamer barcode sequences are configured to indicate internalization of the one or more aptamers into a cell.

In some embodiments, each of the hybridization sequences comprises between 12-20 nucleotides.

Some embodiments further comprise a cell surface molecule bound to the cell surface binding domain. In some embodiments, the cell surface molecule is part of an internalization complex. In some embodiments, the cell surface molecule comprises a receptor tyrosine kinase. In some embodiments, the cell surface molecule comprises a transmembrane protein.

In some embodiments, at least one of the payload binding domains further comprises a payload handle. In some embodiments, the payload handle comprises a protein recognition sequence. In some embodiments, the payload handle comprises a biotinylated structure. In some embodiments, the payload handle comprises a poly-A tail capture sequence.

Some embodiments further comprise a payload bound to the payload binding domain. In some embodiments, the payload comprises a Cas molecule. In some embodiments, the payload comprises a gRNA molecule. In some embodiments, the payload comprises an RNP complex, wherein the RNP complex comprises a Cas molecule associated with a gRNA molecule. In some embodiments, the payload comprises an mRNA molecule. In some embodiments, the payload comprises an oligonucleotide. In some embodiments, the payload comprises a protein. In some embodiments, the payload comprises an RNAi molecule.

In some embodiments, the cell surface binding domain comprises a secondary structure. In some embodiments, the secondary structure comprises a stem loop.

In some embodiments, the cell surface binding domain comprises a tertiary structure.

In some embodiments, the payload binding domain comprises a secondary structure. In some embodiments, the secondary structure comprises a stem loop.

In some embodiments, the payload binding domain comprises a tertiary structure.

Some embodiments further comprise a bead comprising an oligonucleotide, where the oligonucleotide comprises a nucleic acid barcode molecule comprising a bead specific barcode and a capture sequence, where the capture sequence is complementary to at least a portion of at least one of the aptamers. In some embodiments, the oligonucleotide further comprises a unique molecular identifier (UMI)

Aspects of the disclosure comprise a method for internalization of a payload into a cell according to various embodiments. In various embodiments, the method comprises contacting a cell with an internalization complex, the internalization complex comprising (i) a first aptamer comprising a 3′ end, a 5′ end, a cell surface binding domain, and a first hybridization sequence, (ii) a second aptamer comprising a 3′ end, a 5′ end, a payload binding domain, and a second hybridization sequence, and (iii) a payload, wherein the first hybridization sequence is hybridized to the second hybridization sequence, and wherein the payload is bound to the payload binding domain of the second aptamer. In various embodiments, the method comprises binding the cell surface binding domain to a cell surface molecule of the cell. In various embodiments, the method comprises internalizing the internalization complex into a cell.

Some embodiments of these aspects of the disclosure further comprise a step of activating the detectable label upon binding the payload binding domain to the payload. Some embodiments of these aspects of the disclosure further comprise a step of activating the detectable label upon binding the cell surface binding domain to the cell surface molecule. Some embodiments of these aspects of the disclosure further comprise a step of activating a detectable label upon internalization of the internalization complex. In some embodiments, the first aptamer comprises the detectable label. In some embodiments, the second aptamer comprises the detectable label. Some embodiments further comprise a step of emitting a light signal from the detectable label using a fluorescent molecule. Some embodiments further comprise a step of quenching the emitted light signal from the detectable label by interacting a quenching agent with the fluorescent molecule. In some embodiments, the fluorescent molecule is quenched prior to activating. In some embodiments, the fluorescent molecule is quenched after activating. Some embodiments further comprise a step of confirming internalization of the internalization complex.

In some embodiments, at least one of the first aptamer and the second aptamer comprises an aptamer barcode sequence. Some embodiments further comprise a step of sequencing the at least one aptamer barcode sequence.

Some embodiments further comprise a step of confirming correct aptamer pairing. In some embodiments, the step of confirming correct aptamer pairing comprises a fluorescence in situ hybridization method.

Some embodiments further comprise steps of partitioning the cell into a partition with a plurality of nucleic acid barcode molecules, wherein a nucleic acid barcode molecule of the plurality of nucleic acid barcode molecules comprises a partition-specific barcode sequence and a capture sequence that is complementary to at least a portion of at least one of the first aptamer, the second aptamer, and the third aptamer; and hybridizing the capture sequence of the nucleic acid barcode molecule to the at least a portion of the one of the aptamers; and using the nucleic acid barcode molecule and the one of the aptamers to generate a barcoded product comprising the partition-specific barcode sequence or a complement thereof and the aptamer barcode sequence or a complement thereof. In some embodiments, at the step of partitioning the cell, the cell is partitioned with a bead wherein the bead comprises the plurality of nucleic acid barcode molecules. In some embodiments, a nucleic acid barcode molecule of the plurality of nucleic acid barcode molecules further comprises a unique molecular identifier (UMI). Some embodiments further comprise, after the step of using the nucleic acid barcode molecule and one of the aptamers to generate a barcoded product, steps of determining the sequence of the barcoded product or a portion thereof; and confirming internalization of the internalization complex in the cell if the sequence of the barcoded product or portion thereof contains (i) the partition-specific barcode sequence or complement thereof and (ii) the aptamer barcode sequence or complement thereof.

Some embodiments further comprise, prior to the step of contacting a cell with an internalization complex, a step of generating one or more of the aptamers with a polynucleotide synthesis procedure.

Aspects of the disclosure comprise a method for internalization of a plurality of payloads into a cell according to various embodiments. In various embodiments, the method comprises contacting a cell with an internalization complex, the internalization complex comprising (i) a first aptamer comprising a 3′ end, a 5′ end, a cell surface binding domain, and a hybridization sequence, (ii) a second aptamer comprising a 3′ end, a 5′ end, first hybridization sequence, a second hybridization sequence, and a payload binding domain, (iii) a third aptamer comprising a 3′ end, a 5′ end, a payload binding domain, and a hybridization sequence. In various embodiments, the hybridization sequence of the first aptamer is configured to hybridize to the first hybridization sequence of the second aptamer. In various embodiments, the hybridization sequence of the third aptamer is configured to hybridize to the second hybridization sequence of the second aptamer. In various embodiments, the method comprises hybridizing the hybridization sequence of the first aptamer to the first hybridization sequence of the second aptamer and hybridizing the hybridization sequence of the third aptamer to the second hybridization sequence of the second aptamer to create an internalization complex. In various embodiments, the method comprises contacting the payload binding domain of the second aptamer to a first payload and contacting the payload binding domain of the third aptamer to a second payload.

Some embodiments of these aspects of the disclosure further comprise steps of: contacting a cell with the internalization complex; binding the cell surface binding domain to a cell surface molecule; and internalizing the internalization complex. Some embodiments of these aspects of the disclosure further comprise a step of activating a detectable label upon binding one of the payload binding domains to one of the payloads. Some embodiments of these aspects of the disclosure further comprise a step of activating the detectable label upon binding the cell surface binding domain to the cell surface molecule. Some embodiments of these aspects of the disclosure further comprise a step of activating a detectable label upon internalization of the internalization complex. In some embodiments, the first aptamer comprises the detectable label. In some embodiments, the second aptamer comprises the detectable label. In some embodiments, the third aptamer comprises the detectable label. Some embodiments further comprise a step of emitting a light signal from the detectable label using a fluorescent molecule. Some embodiments further comprise a step of quenching the emitted light signal from the detectable label by interacting a quenching agent with the fluorescent molecule. In some embodiments, the fluorescent molecule is quenched prior to activating. In some embodiments, the fluorescent molecule is quenched after activating.

Some embodiments further comprise a step of confirming internalization of the internalization complex.

In some embodiments, at least one of the first aptamer, the second aptamer, and the third aptamer comprises an aptamer barcode sequence. Some embodiments further comprise a step of sequencing the aptamer barcode sequence.

Some embodiments further comprise a step of confirming correct aptamer pairing. In some embodiments, the step of confirming correct aptamer pairing comprises a fluorescence in situ hybridization method.

Some embodiments further comprise steps of: partitioning the cell into a partition with a plurality of nucleic acid barcode molecules, wherein a nucleic acid barcode molecule of the plurality of nucleic acid barcode molecules comprises a partition-specific barcode sequence and a capture sequence that is complementary to at least a portion of at least one of the first aptamer, the second aptamer, or the third aptamer one of the aptamers; and hybridizing the capture sequence of the nucleic acid barcode molecule to the at least a portion of the one of the aptamers; and using the nucleic acid barcode molecule and the one of the aptamers to generate a barcoded product comprising the partition-specific barcode sequence or a complement thereof and the aptamer barcode sequence or a complement thereof. In some embodiments, at the step of partitioning the cell, the cell is partitioned with bead wherein the bead comprises the plurality of nucleic acid barcode molecules. In some embodiments, the nucleic acid barcode molecule of the plurality of nucleic acid barcode molecules further comprises a unique molecular identifier (UMI). Some embodiments further comprise, after the step of using the nucleic acid barcode molecule and one of the aptamers to generate a barcoded product, steps of determining the sequence of the barcoded product or a portion thereof; and confirming internalization of the internalization complex in the cell if the sequence of the barcoded product or portion thereof contains (i) the partition-specific barcode sequence or complement thereof and (ii) the aptamer barcode sequence or complement thereof.

Some embodiments further comprise a step of generating one or more of the aptamers with a polynucleotide synthesis procedure.

Aspects of the disclosure comprise a method for internalization of a plurality of payloads into a cell according to various embodiments. In various embodiments, the method comprise contacting a cell with an internalization complex, the internalization complex comprising (i) a first aptamer comprising a 3′ end, a 5′ end, a cell surface binding domain, a first hybridization sequence, and a second hybridization sequence, (ii) a second aptamer comprising a 3′ end, a 5′ end, a hybridization sequence, and a payload binding domain, and (iii) a third aptamer comprising a 3′ end, a 5′ end, a hybridization sequence, and a payload binding domain. In various embodiments, the first hybridization sequence of the first aptamer is configured to hybridize to the hybridization sequence of the second aptamer. In various embodiments, the second hybridization sequence of the first aptamer is configured to hybridize to the hybridization sequence of the third aptamer. In various embodiments, the method comprises hybridizing the first hybridization sequence of the first aptamer to the hybridization sequence of the second aptamer and hybridizing the second hybridization sequence of the first aptamer to the hybridization sequence of the third aptamer to create an internalization complex. In various embodiments, the method comprises binding the payload binding domain of the second aptamer to a first payload and binding the payload binding domain of the third aptamer to a second payload.

Some embodiments of these aspects of the disclosure further comprise steps of contacting a cell with the internalization complex, binding the cell surface binding domain to a cell surface molecule; and internalizing the internalization complex. Some embodiments of these aspects of the disclosure further comprise a step of activating a detectable label upon binding one of the payload binding domains to one of the payloads. Some embodiments of these aspects of the disclosure further comprise a step of activating a detectable label upon binding the cell surface binding domain to the cell surface molecule. Some embodiments of these aspects of the disclosure further comprise a step of activating a detectable label upon internalization of the internalization complex. In some embodiments, the first aptamer comprises the detectable label. In some embodiments, the second aptamer comprises the detectable label. In some embodiments, the third aptamer comprises the detectable label. Some embodiments further comprise a step of emitting a light signal from the detectable label using a fluorescent molecule. Some embodiments further comprise a step of quenching the emitted light signal from the detectable label by interacting a quenching agent with the fluorescent molecule. In some embodiments, the fluorescent molecule is quenched prior to activating. In some embodiments, the fluorescent molecule is quenched after activating.

Some embodiments further comprise a step of confirming internalization of the internalization complex.

In some embodiments, at least one of the first aptamer, the second aptamer, and the third aptamer comprises an aptamer barcode sequence. Some embodiments further comprise a step of sequencing the aptamer barcode sequence.

Some embodiments further comprise a step of confirming correct aptamer pairing. In some embodiments, the step of confirming correct aptamer pairing comprises a fluorescence in situ hybridization method.

Some embodiments further comprise steps of partitioning the cell into a partition with a plurality of nucleic acid barcode molecules, wherein a nucleic acid barcode molecule of the plurality of nucleic acid barcode molecules comprises a partition-specific barcode sequence and a capture sequence that is complementary to at least a portion of at least one of the first aptamer, the second aptamer, or the third aptamer one of the aptamers; and hybridizing the capture sequence of the nucleic acid barcode molecule to the at least a portion of the one of the aptamers; and using the nucleic acid barcode molecule and the one of the aptamers to generate a barcoded product comprising the partition-specific barcode sequence or a complement thereof and the aptamer barcode sequence or a complement thereof. In some embodiments, at the step of partitioning the cell, the cell is partitioned with a bead wherein the bead comprises the plurality of nucleic acid barcode molecules. In some embodiments, the nucleic acid barcode molecule of the plurality of nucleic acid barcode molecules further comprises a unique molecular identifier (UMI). Some embodiments further comprise, after the step of using the nucleic acid barcode molecule and one of the aptamers to generate a barcoded product, steps of determining the sequence of the barcoded product or a portion thereof; and confirming internalization of the internalization complex in the cell if the sequence of the barcoded product or portion thereof contains (i) the partition-specific barcode sequence or complement thereof and (ii) the aptamer barcode sequence or complement thereof.

Some embodiments further comprise a step of generating one or more of the aptamers with a polynucleotide synthesis procedure.

Aspects of the disclosure comprise a kit for internalizing a payload into a cell according to various embodiments. In various embodiments, the kit comprises first aptamer comprising a 3′ end, a 5′ end, a cell surface binding domain, and a hybridization sequence; and a second aptamer comprising, a 3′ end, a 5′ end, a payload binding domain, and a hybridization sequence. In various embodiments, the hybridization sequence of the first aptamer is configured to hybridize to the hybridization sequence of the second aptamer. In various embodiments, the kit comprises a payload.

In some embodiments of these aspects of the disclosure, the first aptamer further comprises a detectable label. In some embodiments, the second aptamer comprises a detectable label. In some embodiments, the detectable label comprises a fluorescent molecule. In some embodiments, the detectable label comprises a quenching agent configured to interact with the fluorescent molecule and decrease fluorescence.

In some embodiments, at least one of the aptamers comprises an aptamer barcode sequence.

In some embodiments, the payload binding domain further comprises a payload handle. In some embodiments, the payload handle comprises a protein recognition sequence. In some embodiments, the payload handle comprises a biotinylated structure. In some embodiments, the payload handle comprises a poly-A tail capture sequence.

In some embodiments, the payload comprises a Cas molecule. In some embodiments, the payload comprises a gRNA molecule. In some embodiments, the payload comprises an RNP complex, wherein the RNP complex comprises a Cas molecule associated with a gRNA molecule. In some embodiments, the payload comprises an mRNA molecule. In some embodiments, the payload comprises an oligonucleotide. In some embodiments, the payload comprises a protein. In some embodiments, payload comprises an RNAi molecule.

Some embodiments further comprise a bead comprising an oligonucleotide, where the oligonucleotide comprises a nucleic acid barcode molecule comprising a bead specific barcode and a capture sequence, where the capture sequence is complementary to at least a portion of at least one of the aptamers. In some embodiments, the oligonucleotide further comprises a unique molecular identifier (UMI).

Aspects of the disclosure comprise a kit for internalizing a plurality of payloads into a cell according to various embodiments. In various embodiments, the kit comprises a first aptamer comprising a 3′ end, a 5′ end, a cell surface binding domain, and a hybridization sequence; a second aptamer comprising a 3′ end, a 5′ end, a first hybridization sequence, a second hybridization sequence, and a payload binding domain; and a third aptamer comprising, a 3′ end, a 5′ end, a payload binding domain, and a hybridization sequence. In various embodiments, the hybridization sequence of the first aptamer is configured to hybridize to the first hybridization sequence of the second aptamer. In various embodiments, the hybridization sequence of the third aptamer is configured to hybridize to the second hybridization sequence of the second aptamer. In various embodiments, the kit can comprise a plurality of payloads.

In some embodiments of these aspects of the disclosure, the first aptamer further comprises a detectable label. In some embodiments, the second aptamer further comprises a detectable label. In some embodiments, the third aptamer further comprises a detectable label. In some embodiments, the detectable label comprises a fluorescent molecule. In some embodiments, the detectable label comprises a quenching agent configured to interact with the fluorescent molecule and decrease fluorescence.

In some embodiments, at least one of the aptamers comprises an aptamer barcode sequence.

In some embodiments, the payload binding domain further comprises a payload handle. In some embodiments, the payload handle comprises a protein recognition sequence. In some embodiments, the payload handle comprises a biotinylated structure. In some embodiments, the payload handle comprises a poly-A tail capture sequence.

In some embodiments, the payload comprises a Cas molecule. In some embodiments, the payload comprises a gRNA molecule. In some embodiments, the payload comprises an RNP complex, wherein the RNP complex comprises a Cas molecule associated with a gRNA molecule. In some embodiments, the payload comprises an mRNA molecule. In some embodiments, the payload comprises an oligonucleotide. In some embodiments, the payload comprises a protein. In some embodiments, payload comprises an RNAi molecule.

Some embodiments further comprise a bead comprising an oligonucleotide, where the oligonucleotide comprises a nucleic acid barcode molecule comprising a bead specific barcode and a capture sequence, where the capture sequence is complementary to at least a portion of at least one of the aptamers. In some embodiments, the oligonucleotide further comprises a unique molecular identifier (UMI).

Aspects of the disclosure comprise a kit for internalizing a plurality of payloads into a cell according to various embodiments. In various embodiments, the kit comprises a first aptamer comprising a 3′ end, a 5′ end, a cell surface binding domain, a first hybridization sequence, and a second hybridization sequence; a second aptamer comprising, a 3′ end, a 5′ end, a hybridization sequence, and a payload binding domain; and a third aptamer comprising, a 3′ end, a 5′ end, hybridization sequence, and a payload binding domain. In various embodiments, the first hybridization sequence is configured to hybridize to the hybridization sequence of the second aptamer. In various embodiments, the second hybridization sequence is configured to hybridize to the hybridization sequence of the third aptamer. In various embodiments, the kit comprises a plurality of payloads.

In some embodiments of these aspects of the disclosure, the first aptamer further comprises a detectable label. In some embodiments, the second aptamer further comprises a detectable label. In some embodiments, the third aptamer further comprises a detectable label. In some embodiments, the detectable label comprises a fluorescent molecule. In some embodiments, the detectable label comprises a quenching agent configured to interact with the fluorescent molecule and decrease fluorescence.

In some embodiments, at least one of the aptamers comprises an aptamer barcode sequence.

In some embodiments, the payload binding domain further comprises a payload handle. In some embodiments, the payload handle comprises a protein recognition sequence. In some embodiments, the payload handle comprises a biotinylated structure. In some embodiments, the payload handle comprises a poly-A tail capture sequence.

In some embodiments, the payload comprises a Cas molecule. In some embodiments, the payload comprises a gRNA molecule. In some embodiments, the payload comprises an RNP complex, wherein the RNP complex comprises a Cas molecule associated with a gRNA molecule. In some embodiments, the payload comprises an mRNA molecule. In some embodiments, the payload comprises an oligonucleotide. In some embodiments, the payload comprises a protein. In some embodiments, payload comprises an RNAi molecule.

Some embodiments further comprise a bead comprising an oligonucleotide, where the oligonucleotide comprises a nucleic acid barcode molecule comprising a bead specific barcode and a capture sequence, where the capture sequence is complementary to at least a portion of at least one of the aptamers. In some embodiments, the oligonucleotide further comprises a unique molecular identifier (UMI).

Aspects of the disclosure comprise a method of preparing an internalization complex for internalization of a payload into a cell according to various embodiments. In various embodiments, the method comprises hybridizing a hybridization sequence of a first aptamer to a hybridization sequence of a second aptamer. In various embodiments, the first aptamer comprises a 3′ end, a 5′ end, a cell surface binding domain, and the hybridization sequence of the first aptamer. In various embodiments, the second aptamer comprises a 3′ end, a 5′ end, a payload binding domain, and the hybridization sequence of the second aptamer.

Aspects of the disclosure comprise a method of preparing an internalization complex for internalization of a payload into a cell. In various embodiments, the method comprises hybridizing a hybridization sequence of a first aptamer to a first hybridization sequence of a second aptamer. In various embodiments, the method comprises hybridizing a hybridization sequence of a third aptamer to a second hybridization sequence of the second aptamer. In various embodiments, the first aptamer comprises a 3′ end, a 5′ end, a cell surface binding domain, and the hybridization sequence of the first aptamer, wherein the second aptamer comprises a 3′ end, a 5′ end, the first hybridization sequence of the second aptamer, the second hybridization sequence of the second aptamer, and a payload binding domain. In various embodiments, the third aptamer comprises a 3′ end, a 5′ end, the hybridization sequence of the third aptamer, and a payload binding domain of the third aptamer.

Some embodiments of these aspects of the disclosure further comprise binding the payload binding domain of the second aptamer to a payload, thereby preparing the internalization complex for internalization of the payload into the cell. Some embodiments further comprise binding the payload binding domain of the third aptamer to an additional payload.

Aspects of the disclosure comprise a method of preparing an internalization complex for internalization of a payload into a cell. In various embodiments, the method comprises hybridizing a first hybridization sequence of a first aptamer to a hybridization sequence of a second aptamer. In various embodiments, the method comprises hybridizing a second hybridization sequence of the first aptamer to a hybridization sequence of the third aptamer. In various embodiments, the first aptamer comprises a 3′ end, a 5′ end, a cell surface binding domain, the first hybridization sequence, and the second hybridization sequence. In various embodiments, the second aptamer comprises a 3′ end, a 5′ end, the hybridization sequence of the second aptamer, and a payload binding domain. In various embodiments, the third aptamer comprises a 3′ end, a 5′ end, the hybridization sequence of the third aptamer, and a payload binding domain of the third aptamer.

Some embodiments of these aspects of the disclosure further comprise binding the payload binding domain of the second aptamer to a payload, thereby preparing the internalization complex for internalization of the payload into the cell. Some embodiments further comprise binding the payload binding domain of the third aptamer to an additional payload.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF FIGURES

The novel features of the technology are set forth with particularity in the appended claims. A better understanding of the features and advantages of the present technology will be obtained by reference to the following detailed description that sets forth illustrative embodiments, in which the principles of the technology are utilized, and the accompanying drawings (also “Figure” and “FIG.” herein). The accompanying drawings are not intended to be drawn to scale. Like reference numbers and designations in the various drawings indicate like elements. For purposes of clarity, not every component can be labeled in every drawing. In the drawings:

FIG. 1 is a graphical illustration showing aptamer-mediated delivery systems according to some embodiments.

FIG. 2 is a graphical illustration showing a non-branched aptamer folding to create a three-dimensional structure that later binds to a cell surface molecule.

FIG. 3 shows an example of a microfluidic channel structure for partitioning individual analyte carriers according to some embodiments.

FIG. 4 shows an example of a microfluidic channel structure for the controlled partitioning of beads into discrete droplets according to some embodiments.

FIG. 5 is an illustration of an example of a barcode carrying bead according to some embodiments.

FIG. 6 is an illustration of another example of a barcode carrying bead according to some embodiments.

FIG. 7 is a schematic illustration of an example microwell array according to some embodiments.

FIG. 8 is a schematic illustration an example workflow for processing nucleic acid molecules according to some embodiments.

FIG. 9 is a schematic illustration of example labelling agents with nucleic acid molecules attached thereto according to some embodiments.

FIG. 10A is a schematic illustration of example of labelling agents according to some embodiments. FIG. 10B schematically shows another example workflow for processing nucleic acid molecules according to some embodiments. For example, workflow using a primer 1051 having a, e.g., polyT (SEQ ID NO:6), sequence complementary to an RNA molecule 1060, e.g., polyA (SEQ ID NO: 5), sequence. FIG. 10C schematically shows another example workflow for processing nucleic acid molecules, e.g., RNA molecule using its polyA sequence (SEQ ID NO: 5), according to some embodiments.

FIG. 11 schematically shows another example of a barcode-carrying bead according to some embodiments.

FIG. 12 is a schematic illustration showing aptamer-mediated delivery systems according to some embodiments.

FIG. 13 is an illustration depicting an internalization complex comprising two aptamers with a payload bound to one aptamer and a cell surface molecule bound to another aptamer according to various embodiments.

FIG. 14 is an illustration depicting an internalization complex comprising three aptamers in a non-branched format with two payloads each bound to an aptamer and a cell surface molecule bound to another aptamer according to various embodiments.

FIG. 15 is an illustration depicting an internalization complex comprising three aptamers in a branched format with two payloads each bound to an aptamer and a cell surface molecule bound to another aptamer according to various embodiments.

FIG. 16 is a flow diagram depicting a method for generating internalization complexes comprising two aptamers that can deliver a payload to a cell according to various embodiments.

FIG. 17 is a flow diagram depicting a method for generating internalization complexes comprising three aptamers in a non-branched format that can deliver multiple payloads to a cell according to various embodiments.

FIG. 18 is a flow diagram depicting a method for generating internalization complexes comprising three aptamers in a branched format that can deliver multiple payloads to a cell according to various embodiments.

FIG. 19 shows an exemplary microfluidic channel structure for delivering barcode carrying beads to droplets.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes various exemplary embodiments of methods, systems, kits, and compositions for delivering payloads to cells using aptamer-mediated transport pathways (e.g. without the use of lentiviral vectors). Payloads in accordance with embodiments of the invention can comprise any molecule capable of being bound to an aptamer directly or indirectly (e.g. via a payload handle). In some embodiments, gene editing machinery (e.g. CRISPR complexes/components) can be delivered to cells using aptamer-mediated transport pathways. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Headings, e.g., (a), (b), (i) etc., are presented merely for ease of reading the specification and claims. The use of headings in the specification or claims does not require the steps or elements be performed in alphabetical or numerical order or the order in which they are presented.

It should be understood that any uses of subheadings herein are for organizational purposes and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary descriptions of specific features are used, largely for informational purposes, and not in any way to limit the design, subfeature, and functionality of the specifically described feature.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those available and commonly used in the art.

### Definitions

As used herein, the terms “adaptor(s)”, “adapter(s)” and “tag(s)” may be used synonymously. An adaptor or tag can be coupled to a polynucleotide sequence to be “tagged” by any approach, including ligation, hybridization, or other approaches.

As used herein, the term “analyte” refers to a species of interest for detection. An analyte may be biological analyte, such as a nucleic acid molecule or protein. An analyte may be an atom or molecule. An analyte may be a subunit of a larger unit, such as, e.g., a given sequence of a polynucleotide sequence or a sequence as part of a larger sequence. An analyte of the present disclosure includes a secreted analyte, a soluble analyte, and/or an extracellular analyte.

Analytes may be derived from cells and may include at least a portion of a targeting genomic locus, a reverse complement thereof, or a derivative thereof. Analytes may include at least a portion of a tagging genomic locus, a reverse complement thereof, or a derivative thereof.

As used herein, the term “aptamer barcode sequence” refers to a label, or identifier, that conveys information about an aptamer. An aptamer barcode sequence can be part of an aptamer. An aptamer barcode sequence can be independent of an aptamer. An aptamer barcode sequence can comprise an oligonucleotide sequence. An aptamer barcode sequence can be a tag attached to an aptamer. An aptamer barcode sequence can uniquely identify a specific aptamer.

As used herein, the term “barcode” means a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. The nucleic acid barcode molecule may be coupled to or attached to the nucleic acid molecule comprising the nucleic acid sequence. For example, in the methods and compositions described herein, hybridization and reverse transcription of a nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

As used herein, the term “bead” generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “binding agent” generally refers to a molecule capable of binding to one or more other molecules (e.g., analytes, receptors, other binding agents, etc.) and that comprises one or more portions. In some cases, a binding agent comprises at least one, at least two, at least three, or at least four portions. Each portion may comprise a polypeptide. The polypeptide of a specific portion may be capable of binding one or more molecules. For example, a polypeptide of a specific portion may bind to a molecule located on a surface of a cell, such as a cell surface protein or receptor (e.g., a CD surface marker such as CD45). A polypeptide of another portion of a binding agent may bind a molecule that may be secreted from a cell (e.g., T cell, B-cell, dendritic cell, etc.). The one or more portions of a binding agent may be directly or indirectly linked to, conjugated to, or fused to one another. For example, a first portion of a binding agent may be directly or indirectly linked to, conjugated to, or fused to a second portion of the binding agent. Moreover, the terms “binding agent,” “polypeptide,” and “antibody” may be used interchangeably herein.

As used herein, the term “biological particle” generally refers to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

As used herein, the term “cell bead” generally refers to a hydrogel, polymeric, or crosslinked material that comprises (e.g., encapsulates, contains, etc.) a biological particle (e.g., a cell, a nucleus, a fixed cell, a cross-linked cell), a virus, components of or macromolecular constituents of or derived from a cell or virus. For example, a cell bead may comprise a virus and/or a cell. In some cases, a cell bead comprises a single cell. In some cases, a cell bead may comprise multiple cells adhered together. A cell bead may include any type of cell, including without limitation prokaryotic cells, eukaryotic cells, bacterial, fungal, plant, mammalian, or other animal cell types, mycoplasmas, normal tissue cells, tumor cells, immune cells, e.g., a T-cell (e.g., CD4 T-cell, CD4 T-cell that comprises a dormant copy of human immunodeficiency virus (HIV)), a B cell, or a dendritic cell, a fixed cell, a cross-linked cell, a rare cell from a population of cells, or any other cell type, whether derived from single cell or multicellular organisms. Furthermore, a cell bead may comprise a live cell, such as, for example, a cell may be capable of being cultured. Moreover, in some examples, a cell bead may comprise a derivative of a cell, such as one or more components of the cell (e.g., an organelle, a cell protein, a cellular nucleic acid, genomic nucleic acid, messenger ribonucleic acid, a ribosome, a cellular enzyme, etc.). In some examples, a cell bead may comprise material obtained from a biological tissue, such as, for example, obtained from a subject. In some cases, cells, viruses or macromolecular constituents thereof are encapsulated within a cell bead. Encapsulation can be within a polymer or gel matrix that forms a structural component of the cell bead. In some cases, a cell bead is generated by fixing a cell in a fixation medium or by cross-linking elements of the cell, such as the cell membrane, the cell cytoskeleton, etc.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

As used herein, the terms “coupled,” “linked,” “conjugated,” “associated,” “attached,” “connected” or “fused,” may be used interchangeably and generally refer to one molecule (e.g., polypeptide, receptor, analyte, etc.) being attached or connected (e.g., bound) to another molecule (e.g., polypeptide, receptor, analyte, etc.).

Two molecules may be “covalently linked” or “covalently attached” to one another when at least one atom in the first molecule shares at least one electron pair with at least one atom in the second molecule. In some embodiments, a covalent linkage between two molecules can involve one or more intermediary molecules. For example, a first molecule and a second molecule may be considered covalently linked, if they are each covalently linked to a linker molecule. In such a circumstance, all three molecules (the first molecule, the second molecule, and the linker molecule) are covalently linked to one another.

As used herein, the term “detectable label” generally mean anything that can be detected. More specifically, detectable labels can comprise fluorescent molecules such as fluorophores or barcodes. Detectable labels can be coupled to macromolecular constituents such as proteins, antibodies, aptamers, or amino acids using enzymatic, chemical or other known labeling methods. Detectable labels can be encoded into genomes using gene editing technologies such as CRISPR and then expressed or chemically added/coupled onto existing macromolecular constitutes. Detectable labels can be exogenous or endogenous. In some applications, detectable labels can comprise quenching agents for reducing a signal intensity being emitted by another molecule (e.g. a fluorophore).

As used herein, the term “DNA” ((deoxyribonucleic acid) means a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

As used herein, the term “macromolecular constituent” generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

As used herein, the term “molecular tag” generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

As used herein, the term “partition” generally refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

As used herein, the term “payload” generally means any molecule that can be bound to another molecule for delivery to a specified location (e.g. the interior of a cell, nucleus, or mitochondria). A payload can comprise oligonucleotides or peptides. In some embodiments disclosed herein, aptamers can selectively bind payload(s) including internalization complexes and/or CRISPR complexes or individual CRISPR components for delivery into the interior region of a cell (e.g. cytoplasm or nucleoplasm).

As used herein, the terms “polynucleotide”, “nucleic acid”, or “oligonucleotide” refer to a non-branched polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

As used herein, the term “polynucleotide synthesis” or “oligonucleotide synthesis” refers to generation of oligonucleotides through artificial means. In some embodiments, polynucleotide synthesis can be organochemical synthesis of oligonucleotides. In some embodiments, polynucleotide synthesize can comprise methods using enzymes (e.g. polymerase). Polynucleotide synthesis can comprise techniques for producing custom sequences of oligonucleotides. Polynucleotide synthesis can comprise producing polymers of nucleotides. In some embodiments, a polynucleotide synthesis process can comprise using a phosphoramidite method and phosphoramidite building blocks. In some embodiments, polynucleotide synthesis can comprise synthesis of aptamers having predetermined sequences.

As used herein, the term “quenching agent” generally refers to any molecule that can decrease the fluorescence intensity of a substance. Processes that result in quenching comprise energy transfer, complex-formation, and collisional quenching.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

As used herein, the term “sequencing” generally refers to methods and technologies for determining the sequence of nucleotide bases on one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

As used herein, the term “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those available and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those available and commonly used in the art.

The terms “a,” “an,” and “the,” as used herein, generally refers to singular and plural references unless the context clearly dictates otherwise. “A and/or B” is used herein to include all of the following alternatives: “A”, “B”, “A or B”, and “A and B”.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

### I. Overview

Aptamer-mediated delivery systems can be used to introduce payload molecules into cells. Such delivery systems overcome several drawbacks in the current state of the art, including low transfection efficiencies, low cell viability after transfection, and other issues reducing the success rate of introducing material into cells. Lentiviral delivery systems exemplify these drawbacks and are most commonly used in gene therapy delivery systems. Aptamer delivery systems can deliver gene editing expression systems comprising components (e.g. RNP complexes) and subcomponents (e.g. gRNA molecules and endonuclease) of such systems. Expression of the CRISPR/Cas machinery can then be used to modify the genomes of cells.

In various embodiments, aptamers can behave similarly to antibodies in that they can bind a variety of targets based on their sequences, secondary structures, tertiary structures, and/or higher order structures. Further, aptamers can be synthesized using known oligonucleotide sequencing systems and methods, which makes them ideal for experimentation and optimization. In various embodiments, aptamers can hybridize to other oligonucleotide sequences in single-stranded regions as well as to themselves to create stem loops and other secondary structures. Aptamers can comprise many tertiary structures that can bind protein molecules with high affinity and specificity using molecular interactions, such as, e.g., van der Waals interactions, hydrogen bonding, and electrostatic interactions, to most small-molecule, peptide, or protein targets, with KD values ranging from 10 pM to 10 nM. Aptamers can range in length from very short sequences (e.g. 50-60 nucleotides) to longer sequences (e.g. 300 nucleotides).

### Aptamer Structure and Function

Naturally-occurring aptamers can be found in riboswitches, which are regulatory segments of mRNA. This gives mRNA the ability to regulate its own activity by binding to a variety of different regulatory elements.

Aptamers can also be produced artificially with relative ease because they comprise single-stranded oligonucleotides. Aspects of the disclosure include aptamers having both lower and higher order structures. Systematic evolution of ligands by exponential enrichment (SELEX) is a combinatorial chemistry technique that can be used for producing oligonucleotides (e.g. aptamers) that specifically bind to target ligands. A non-limiting example of aptamer production using the SELEX technique includes production of spinach aptamers. Lower order structures can allow aptamers to hybridize to other oligonucleotides, including other aptamers. Higher order structures can allow aptamers to bind to target molecules, such as cell surface molecules.

Example sequences of aptamers produced using the SELEX technique include Biotin-TACCCCTTTAATCCCAAACCC (SEQ ID NO: 1) (Biotin-Rb1), Biotin-S-S-S-S-ATCTAACTGCTGCGCCGC CGGGAAAATACTGTACGGTTAGA (SEQ ID NO: 2) (Biotin-sgc8c), and Biotin-S-S-ACTTATTCAATTCCT GTGGGAAGGCTATAGAGGGGCCAGTCTATGAATAAG (SEQ ID NO: 3) (Biotin-sgc3b) are described in J Proteome Res. 2008 May; 7(5): 2133-2139, the disclosure of which is hereby incorporated by reference in its entirety.

In various embodiments, aptamer libraries can be generated, and target specificity can be determined and optimized for use in a variety of different applications. A non-limiting example may comprise generation of an aptamer capable of binding to a specific payload to be internalized into a cell. The aptamer in the example can also include a hybridization sequence to bind other aptamers or may comprise a higher order structure for binding either directly or indirectly with a cell surface molecule such as a transmembrane protein. The embodiments described herein build upon these themes and others.

In various embodiments, internalization complexes can be internalized via pinocytosis. In various embodiments, internalization complexes can be internalized via receptor-mediated pathways. In some embodiments, receptor-mediated pathways can be pH-mediated, allowing for control of internalization. In some embodiments, internalization can be impacted by environmental conditions (e.g. temperature).

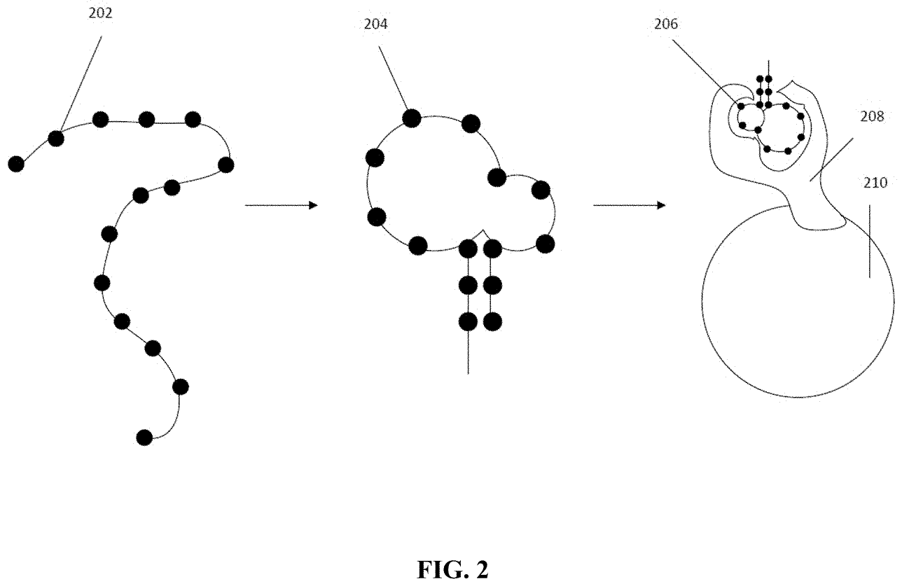

FIG. 2 is a graphical illustration showing a single-stranded oligonucleotide as an unfolded aptamer sequence 202 folding to create a three-dimensional structure that can bind to a target 208 (e.g., a payload, a cell surface molecule of a cell) according to various embodiments. In various embodiments, an unfolded aptamer sequence 202 can be artificially synthesized using known methods in the art. The unfolded aptamer sequence 202 can be placed in an appropriate solution causing the unfolded aptamer sequence 202 to become a folded aptamer 204, thereby, producing pre-determined secondary and tertiary structures. In various embodiments, aptamers can be pH sensitive, and their conformations can be modulated by adjusting a pH. In some embodiments, libraries of oligonucleotides can be generated to produce different folded aptamers 204 comprising any desired secondary, tertiary, or higher order structures.

In various embodiments, a folded aptamer can become a bound aptamer 206. In such embodiments, binding involves a folded aptamer 204 becoming a bound aptamer 206 to a target 208. In some embodiments, the target 208 comprises a cell surface transmembrane protein embedded in a target cell 201. In the various embodiments disclosed herein, the properly folded aptamer 204 can also bind payloads or other aptamers that can also be bound and internalized into the target cell 210.

### Aptamer-Mediated Payload Delivery Systems and Methods

As described above, aptamers have the ability to hybridize or bind a variety of different target molecules. In some embodiments, a first aptamer can hybridize or bind to a second aptamer, including but not limited to, themselves.

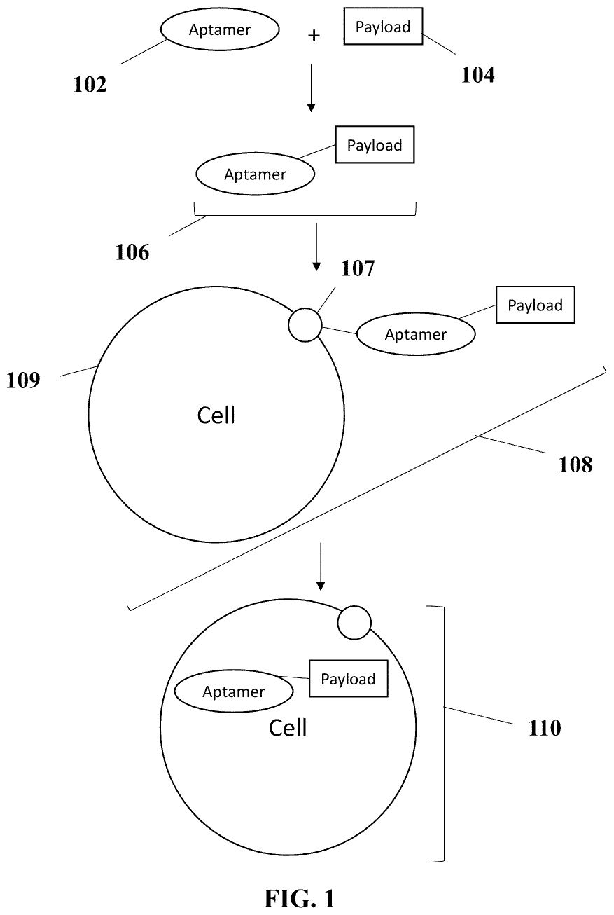

In a variety of embodiments described herein, an internalization complex can be or comprise an aptamer-payload complex 106 (FIG. 1).

FIG. 1 depicts exemplary aptamer-mediated delivery systems according to some embodiments. In various embodiments, an aptamer 102 can serve as a carrier to transport a payload 104. In such embodiments, an aptamer 102 can bind to a payload to form an aptamer-payload complex 106.

An internalization complex (e.g., an aptamer-payload complex 106) can comprise a single aptamer 102 and a single payload 104. In some embodiments, an aptamer-payload complex 106 can comprise more than one aptamer 102. In some embodiments, an aptamer-payload complex 106 can comprise more than one payload 104. In several embodiments, aptamer-payload complexes 106 can comprise any number of aptamers 102 and any number of payloads 104. In such embodiments, aptamers 106 can be hybridized to one another and the aptamers 106 can bind to a variety of different or the same payload 104 molecules. In a variety of embodiments, other molecules may be present to mediate the interactions between the aptamers 102 and payloads 104 in the aptamer-payload complexes 106 such as payload handles.

In various embodiments, an internalization complex (e.g., aptamer-payload complex 106) can contact a cell 109. In some embodiments, the internalization complex (e.g., aptamer-payload complex 106) can contact a cell 109 thereby forming a complex 108 of the cell 109 and the internalization complex. The complex 108 can comprise one or more aptamers 102, one or more payloads 104, and a cell surface molecule 107. In some embodiments, internalization complex can comprise an aptamer-payload complex 106. In other embodiments, internalization complex can comprise an aptamer-payload complex 106 and a cell surface molecule 107. In some embodiments, complex 108 can comprise an internalization complex. In various embodiments, a cell surface molecule 107 can comprise one or more proteins, including transmembrane proteins. In various embodiments, a cell surface molecule 107 can comprise a carbohydrate. In various embodiments, a cell surface molecule 107 can comprise any molecule associated with a cell membrane. In some embodiments, a cell surface molecule 107 can comprise a tyrosine kinase receptor.

Aspects of the disclosure include methods that result in an interaction between a cell surface molecule 107 and an aptamer 102, which interaction facilitates internalization of an internalization complex (e.g., aptamer-payload complex 106, complex 108) into the cell. In some aspects, a method results in internalization 110, wherein a payload 104 can be transported across a cell membrane or a nuclear membrane of cell 109.

### Aptamer-Mediated CRISPR Component Delivery Systems and Methods

Various applications described herein relate to internalization of payload molecules comprising gene editing machinery.

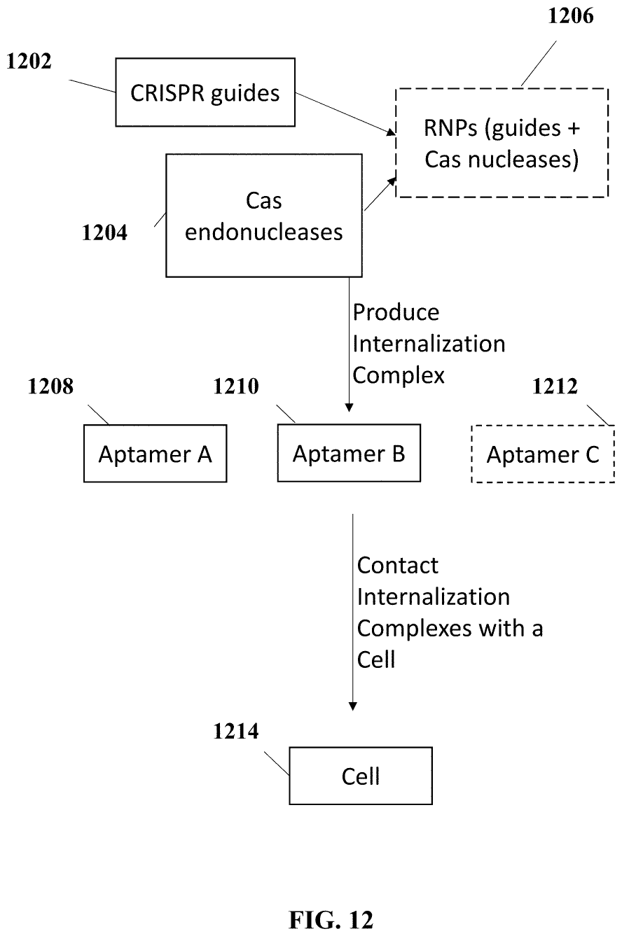

FIG. 12 is a graphical illustration showing aptamer-mediated delivery systems and methods according to various embodiments. In various embodiments, CRISPR components (e.g. RNPs, gRNAs, and endonucleases) can be engineered and manufactured for later delivery into a cell's interior (e.g. cytoplasm, nucleoplasm, mitochondrial matrix, etc.) via aptamer delivery systems. CRISPR guides 1202 and endonucleases (e.g. Cas-9) 1204 can also be engineered and manufactured and then the individual components can remain separated or they can be optionally be combined into an RNP complex 1206 prior to delivery to a cell 1214. In various embodiments, production of CRISPR guides can make use of PERTURB-seq protocols customized for specific targets of interest. In various embodiments, Cas endonucleases 1204 can be produced to comprise tags such as His tags or any kind of recognition tag. Further, there are many Cas endonucleases known in the art that can be utilized in connection with this disclosure. An example sequence of an aptamer including an anti-His binding domain includes 5′-TGA CTG ATT TAC GGC TAT GGG TGG TCT GGT TGG GAT TGG CCC CGG GAG CTG GC-3′ (SEQ ID NO: 4) as described in “A bispecific circular aptamer tethering a built-in universal molecular tag for functional protein delivery,” Chem. Sci., 2020, 11, 9648, the disclosure of which is herein incorporated by reference in its entirety for all purposes.

In various embodiments, any of the aptamers 1208, 1210, and optionally 1212 discussed herein can be selected or designed for a specified purpose. For example, in various embodiments Aptamer A 1208 can be selected or designed to bind to a specific transmembrane molecule (e.g. a tyrosine kinase receptor) of a cell, Aptamer B 1210 can be selected or designed to comprise a hybridization region for hybridizing to Aptamer A 1208. Aptamer B can further comprise a payload binding domain. Aptamer B can optionally further comprise a hybridization region for hybridizing to a hybridization sequence on Aptamer C 1212. Aptamer C 1212 can comprise a payload binding domain. The example described above, and similar embodiments, allow for generation of internalization complexes that can comprise multiple aptamers that enable delivery of multiple payloads in a single internalization event. In some embodiments, an internalization event involves an internalization complex (e.g., comprising one or more aptamers and one or more payloads) binding to a cell surface molecule on a cell, such as a transmembrane protein, and being delivered into the interior of the cell 1214. In some embodiments, the binding of the internalization complex to the cell surface molecule induces internalization of the cell surface molecule with the internalization complex bound thereto. In various embodiments, payloads can comprise proteins, oligonucleotides, or any molecule that can be bound to an aptamer.

In various embodiments, Aptamer A 1208 can include two hybridization sequences, wherein one of the hybridization sequences can bind to a hybridization sequence on Aptamer B 1210 and another hybridization sequence can bind to a hybridization sequence on Aptamer C 1212. In various embodiments, Aptamers B 1210 and C 1212 comprise payload binding domains and can bind payloads. In various embodiments, the payloads can comprise guide RNA molecules and Cas endonucleases 1204. In various embodiments, the payloads can comprise different RNP complexes.

In various embodiments, Aptamer A 1208 may be designed or selected to comprise a hybridization sequence that hybridizes with a hybridization sequence of Aptamer B 1210. In such an embodiment, an internalization complex can consist of two aptamers 1208, 1210 and can be internalized. In such embodiments, Aptamer B 1210 can comprise a payload-binding domain.

In various embodiments, Aptamer A 1208 can comprise both a transmembrane-binding domain and a payload-binding domain. In such embodiments, a single aptamer can be used to bind to a payload, as well as bind to a transmembrane protein, and then be internalized. In such embodiments, the internalization complex can consist of a single aptamer and a single payload.

In various embodiments, the internalization complexes described above are internalized into a cell 1214 where genome editing can occur using the payloads bound to one or more aptamers.

In various embodiments, payloads can comprise any molecule that can both bind to an aptamer and be internalized into a cell 1214.

### II. Compositions and Systems

Provided herein are compositions and systems for delivering payloads into the interior of cells (e.g. cytoplasm or nucleoplasm). In various embodiments, payloads can comprise a Cas molecule, a gRNA molecule, an RNP complex (e.g. gRNA bound to a Cas molecule), an mRNA molecule, an oligonucleotide such as RNA, miRNA, lncRNA, siRNA, ssDNA, or dsDNA, or a polypeptide. In various embodiments, payloads can comprise a drug molecule, a medical treatment molecule, a toxic molecule, and/or a detectable label.

### Two Aptamer Internalization Complexes

Aspects of the disclosure include aptamer internalization complexes wherein two aptamers are hybridized to one another via their respective hybridization sequences. In some embodiments, one of the aptamers binds to a molecule on the surface of a cell and the other binds to one or more payloads.

In various embodiments, the aptamer that binds to a molecule on the surface of the cell may bind to a carbohydrate, a lipid or a protein on the surface of the cell. In some instances, the molecule on the surface of the cell, to which the aptamer may bind, may be a protein, e.g., receptor, transmembrane protein, protein channel, protein pump, or carrier protein. In some instances, the aptamer may bind a protein on the surface of the cell and be internalized by the cell. Non-limiting examples of aptamers that bind to a protein on the surface of the cell, and are internalized, include: A10 and A10-3.2. which specifically bind to prostate-specific membrane antigen (PSMA; Dassie et al., Nat. Biotechnol. 27(2009):839-849); GL56, which specifically binds to insulin receptor (IR; Iaboni et al., Nucleic Acids 5(2016):e365); sgc8, which specifically binds to protein tyrosine kinase-7 (PTK7) also known as colon carcinoma kinase-4 (Shangguan et. al., J. Proteome Res. 7(2008):2133-2139); c2 and c2 min, which specifically bind to human transferrin receptor (Wilner et al., Molecular Therapy-Nucleic Acids, 1(2012):e21); E07, which specifically binds to epidermal growth factor receptor (EGFR; Li et al., PLOS One 6:(2011):e20299); GL21.T, which specifically binds to Axl tyrosine kinase receptor (Cerchia et al., Molecular Therapy 20(2012):2291-2303); DY-647, which specifically binds to epithelial cell adhesion molecule (EpCAM; Shigdar et al., Cancer Science 102(2011):991-998); AIR-3A, which specifically binds to interleukin-6 receptor (IL-6R; Meyer et al., RNA Biology 9(2012):67-80); and Gint4.T, which specifically binds to platelet-derived growth factor receptor beta (PDGFRb; (Romanelli et al., PLOS One 13(2018): e0193392). All references incorporated by reference in their entirety for all purposes. An aptamer that binds to a molecule on the surface of a cell may include a cell surface binding domain engineered from the appropriate domain of any of the aforementioned aptamers for the purpose of targeting the same protein as that aptamer. An aptamer that binds to a molecule on the surface of a cell, may include a cell surface binding domain prepared from, and selected, using the SELEX technique (described earlier herein), as were many of the aforementioned aptamers.

In various embodiments the aptamer that binds to one or more payloads may bind to, for example, a drug molecule, medical treatment molecule, toxic molecule, detectable label, polypeptide such as a Cas molecule, polynucleotide and/or any other payload molecule.

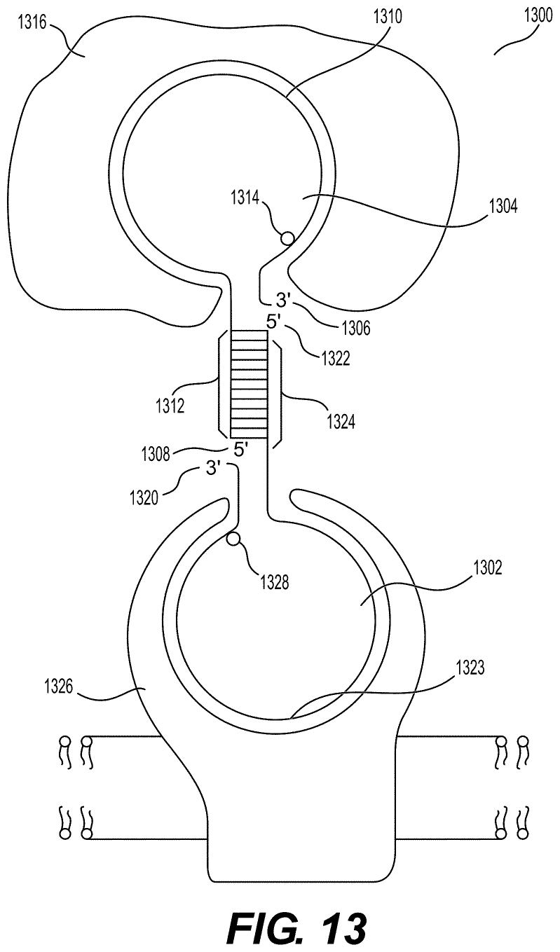

FIG. 13 is an illustration depicting an internalization complex 1300 comprising two aptamers with a payload bound to one aptamer and a cell surface molecule bound to another aptamer according to various embodiments.

In various embodiments, a first aptamer 1302 can hybridize to a second aptamer 1304 using hybridization sequences 1312, 1324. In various embodiments, a first aptamer 1302 can comprise a 3′ end 1320, a 5′ 1322 end, a hybridization sequence 1324, and a cell surface binding domain 1323. In various embodiments, a second aptamer 1304 can comprise a 3′ end 1306, a 5′ end 1308, and a payload binding domain 1310.

In various embodiments, the hybridization binding sequence 1324 of the first aptamer can be configured to hybridize with the hybridization sequence 1312 of the second aptamer.

In various embodiments, a first aptamer 1302 can comprise a detectable label 1328. In various embodiments, a second aptamer 1304 can comprise a detectable label 1314. For clarity, any of the aptamers described herein can comprise a detectable label (e.g., 1314, 1328). In various embodiments, a detectable label (e.g., 1314, 1328) can comprise a fluorescent molecule. In various embodiments, a detectable label can comprise a quenching agent configured to interact with the fluorescent molecule to decrease or limit fluorescence of the fluorescent molecule.

In various embodiments, a detectable label (e.g., 1314, 1328) can be activated or deactivated (e.g. triggered) upon internalization of at least one of the aptamers (e.g., 1302, 1304) into a cell. In various embodiments, the detectable label 1314 can be activated or deactivated upon the second aptamer 1304 binding a payload 1316 or payload molecule. In various embodiments, the detectable label 1328 can be activated or deactivated upon binding a cell surface molecule 1326.

In various embodiments, a detectable label (e.g., 1314, 1328) may generate a signal or create fluorescence as a default state. For example, a detectable label (e.g., 1314, 1328) can be detectable until the default state changes, such as one of the aptamers (e.g., 1302, 1304) going from an unbound state to a bound state or the internalization complex 1300 moving from outside of a cell to the cell's interior (e.g. release from the cell surface molecule). In various embodiments, a detectable label may not generate (e.g. be quenched) a signal or create fluorescence as a default state. For example, a detectable label (e.g., 1314, 1328) may not be detectable until the default state changes, such as one of the aptamers (e.g., 1302, 1304) going from an unbound state to a bound state or the internalization complex 1300 moving from outside of a cell to the cell's interior. In various embodiments, fluorescence resonance energy transfer (FRET), resonance energy transfer (RET) or electronic energy transfer (EET) can be used to detect molecules in proximity to one another (e.g. unbound state to bound state). Accordingly, aspects of the disclosure include methods that involve detecting a change in a signal emitted from a detectable label, such as, e.g., a change in a fluorescent molecule from emitting highly detectable levels of fluorescence to lower levels of fluorescence, or vice versa (i.e., a change in a fluorescent molecule from emitting low or undetectable levels of fluorescence to highly detectable levels of fluorescence). An aptamer that comprises a detectable label may be an aptamers having a domain, e.g., via having been engineered to include a domain, or variant of a domain of any aptamer, e.g., a spinach aptamer, broccoli aptamer, mango aptamer, corn aptamer, or any variant thereof, (e.g. baby spinach or spinach 2 aptamer), capable of binding a detectable label.

In various embodiments, one or more of the aptamers 1302, 1304 may comprise an aptamer barcode sequence. For clarity, any of the aptamers described herein can comprise an aptamer barcode sequence. In various embodiments, an aptamer barcode sequence can be located anywhere with the sequence of an aptamer 1302, 1304. In various embodiments, an aptamer barcode sequence can be located adjacent to the 3′ end 1306, 1320 of an aptamer 1302, 1304. In various embodiments, an aptamer barcode sequence can be located adjacent to the 5′ end 1306, 1320 of an aptamer 1302, 1304.

In various embodiments, aptamer barcode sequences can be positioned or located within about 50 nucleotides from a 3′ or 5′ end of the aptamer 1302, 1304, such as 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotides from the 3′ or 5′ end of an aptamer 1302, 1304. In some embodiments, an aptamer barcode sequence can be positioned at the 3′ or 5′ end of the aptamer 1302, 1304.

In various embodiments, an aptamer barcode sequence can be located at a position on an aptamer where there is no secondary structure.

In various embodiments, the hybridization sequence 1312 of the first aptamer 1302 is configured to hybridize to the hybridization sequence 1324 of the second aptamer 1304. In various embodiments, the hybridization sequence 1312 of the first aptamer 1302 can comprise a first aptamer barcode sequence the hybridization sequence 1324 of the second aptamer 1304 can comprise a second aptamer barcode sequence. In various embodiments, the hybridization sequence 1312 of the first aptamer 1302 can comprise an aptamer barcode sequence. In various embodiments, the hybridization sequence 1324 of the second aptamer 1304 can comprise an aptamer barcode sequence. In various embodiments, pairing of aptamer barcode sequences can be detectable and indicates correct aptamer pairing (e.g. the first aptamer is hybridized to the second aptamer). In various embodiments, aptamer barcodes can be used to indicate that internalization of one or more aptamers or internalization complexes has occurred.

In various embodiments, a hybridization sequence 1324 of the first aptamer 1302 can be positioned between a cell surface binding domain 1323 and a 3′ end 1320 of the first aptamer 1302.

In various embodiments, a hybridization sequence 1324 of a first aptamer 1302 can be positioned between the cell surface binding domain 1323 and the 5′ end 1322 of the first aptamer 1302.

In various embodiments, a hybridization sequence 1312 of a second aptamer 1304 can be positioned between the payload binding domain 1310 and the 3′ end 1306 of the second aptamer 1304.

In various embodiments, a hybridization sequence 1312 of a second aptamer 1304 can be positioned between the payload binding domain 1310 and the 5′ end 1308 of the second aptamer 1304.

In various embodiments, hybridization sequences 1324, 1312 can range in length from 12-20 nucleotides. In various embodiments, hybridization sequences 1324, 1312 can range in length from 8-30 nucleotides.

In various embodiments, a cell surface molecule 1326 can be bound to a cell surface binding domain 1323. In various embodiments, a cell surface molecule 1326 can be part of the internalization complex 1300. In some embodiments, cell surface molecule 1326 is a cell surface protein, e.g., an endogenous cell surface protein that can be internalized, e.g., by endocytosis. In some embodiments, the binding of the internalization complex to the cell surface molecule induces internalization of the cell surface molecule with the internalization complex bound thereto. In various embodiments, a cell surface molecule 1326 can comprise a receptor tyrosine kinase, e.g., PTK7, Axl, PDGFRb, IR, or EGFR. In various embodiments, a cell surface molecule 1326 can comprise a transmembrane protein, e.g., IL-6R, EpCAM or PSMA.

In various embodiments, a payload binding domain 1310 can comprise a payload handle. In various embodiments, a payload handle can act as a universal adaptor allowing one or more standard or known aptamer configurations to be used to bind as many different types and variants of payloads as necessary for internalization.

In various embodiments, a protein recognition sequence can be used as a payload handle. In various embodiments, a protein recognition sequence can be common throughout proteins of a specified class or type. In various embodiments, a protein recognition sequence can be designed to recognize a group of proteins of interest. In various embodiments, a protein recognition sequence of an aptamer can be designed to bind to a protein sequence or higher order structure inherent to a payload 1316. In various embodiments, a protein recognition sequence of an aptamer can be designed to bind to a protein sequence or higher order structure that has been artificially generated or added to a payload.

In various embodiments, a payload binding domain 1310 can comprise a first payload handle including biotin and a payload 1316 can comprise a second payload handle including a biotin-binding protein (e.g. avidin or streptavidin). In various non-limiting examples, the payload 1316 can be any molecule capable of binding the biotin-binding protein (e.g. several examples are described herein).

In various embodiments, a payload binding domain 1310 can comprise a first payload handle including a biotin-binding protein (e.g. avidin or streptavidin) and a payload 1316 can comprise a second payload handle including biotin. In various non-limiting examples, the payload 1316 can be any molecule capable of binding to biotin (e.g. several examples are described herein).

In various embodiments, a payload 1316 can comprise a payload handle. In various embodiments, a payload binding domain 1310 can comprise a payload handle. In various embodiments, both a payload 1316 and a payload binding domain 1310 can comprise payload handles that can be configured to interact with one another and act as universal adaptors. A skilled artisan will appreciate that a variety of payload handle combinations and permutations can be utilized for general and specific purposes.

In various embodiments, a payload handle can be covalently bound to a payload binding domain 1310. In various embodiments, a payload handle can be non-covalently bound to a payload binding domain 1310.

In various embodiments, a payload handle can be covalently bound to a 1316 payload. In various embodiments, a payload handle can be non-covalently bound to a payload 1316.

In various embodiments, an internalization complex 1300 can comprise a payload-binding domain 1310 bound to a payload 1316. In various embodiments, a payload 1316 can comprise a Cas molecule. In various embodiments, a payload 1316 can comprise a gRNA molecule. In various embodiments, a payload 1316 can comprise an RNP complex and the RNA complex can comprise a Cas molecule and a gRNA molecule. In various embodiments, a payload 1316 can comprise an mRNA molecule. In various embodiments, a payload 1316 can comprise a nucleotide. In various embodiments, a payload 1316 can comprise a protein. In various embodiments, a payload 1316 can comprise an RNAi molecule.

In various embodiments, a cell surface-binding domain 1323 can comprise a secondary structure. In various embodiments, the secondary structure can comprise one or more stem loops. In various embodiments, a cell surface-binding domain 1323 can comprise a tertiary structure.

In various embodiments, a payload-binding domain 1310 can comprise a secondary structure. In various embodiments, the secondary structure can comprise one or more stem loops. In various embodiments, a payload-binding domain 1310 can comprise a tertiary structure.

### Three Aptamer Internalization Complexes

Aspects of the disclosure comprise compositions having three aptamers hybridized to one another. Such embodiments can allow internalization complexes to carry multiple payloads capable of being internalized in a single internalization event.

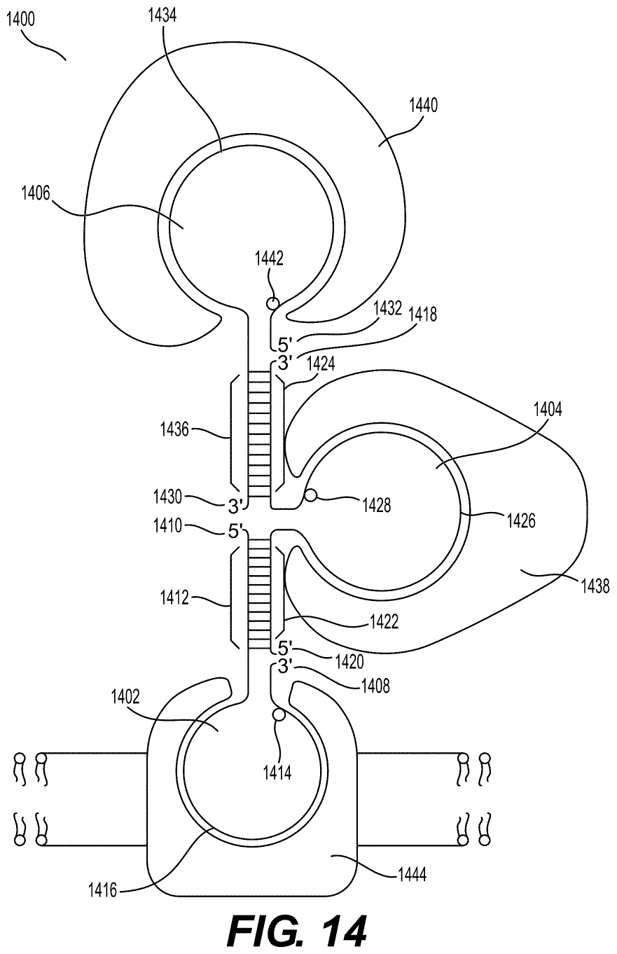

FIG. 14 is an illustration depicting an internalization complex 1400 comprising three aptamers in a non-branched format with two payloads each bound to an aptamer and a cell surface molecule bound to another aptamer according to various embodiments.

In various embodiments, compositions for internalizing a plurality of payloads into a cell are described herein. In a non-limiting example, three aptamers can be hybridized to one another to generate a non-branched format. In some embodiments, a first aptamer 1402 can be hybridized to a second aptamer 1404 and the second aptamer 1404 can be hybridized to a third aptamer 1406.

In various embodiments, a first aptamer 1402 can comprise a 3′ end 1408 and a 5′ end 1410. In various embodiments, a first aptamer 1402 can comprise a cell surface binding domain 1416 configured to bind with a cell surface molecule 1444. Aptamers having cell surface binding domains, and design of aptamers having cell surface binding domains, have been described herein. In various embodiments, a first aptamer 1402 can comprise a detectable label 1414. In some embodiments, a first aptamer 1402 can comprise a first aptamer hybridization sequence 1412 that can hybridize with a first hybridization sequence 1422 of a second aptamer 1404.

In various embodiments of the non-branched aptamer format, a second aptamer 1404 can comprise a 3′ end 1418 and a 5′ end 1420. In various embodiments, a second aptamer 1404 can comprise a payload binding domain 1426 configured to bind a payload 1438. In some embodiments, a second aptamer 1404 can comprise a detectable label 1428. In various embodiments, a second aptamer 1404 can comprise a second hybridization sequence 1424 configured to hybridize with a hybridization sequence 1436 of a third aptamer 1406.

In various embodiments, a third aptamer 1406 can comprise a 3′ end 1430 and a 5′ end 1432. In some embodiments, a third aptamer 1406 can comprise a payload binding domain 1434 configured to bind a payload 1440. In various embodiments, a third aptamer 1406 can comprise a detectable label 1442.

In various embodiments, detectable label (e.g., 1414, 1428, 1442) can comprise a fluorescent molecule. In some embodiments, the detectable label can comprise a quenching agent configured to interact with the fluorescent molecule and decrease fluorescence. In some embodiments, a detectable label (e.g., 1414, 1428, 1442) can be activated upon internalization of at least one of the aptamers (e.g., 1402, 1404, 1406) into a cell. In some embodiments, a detectable label (e.g., 1414, 1428, 1442) can be activated upon binding a payload to at least one of the aptamers (e.g., 1402, 1404, 1406). In some embodiments, a detectable label (e.g., 1414, 1428, 1442) can be activated upon binding a cell surface molecule (e.g., 1444) to at least one of the aptamers. As discussed herein, aptamers, e.g., first and/or second and/or third aptamers, that may comprise the detectable label may be aptamers that include, or may be engineered to include, a domain (or variant of a domain) such as that of a spinach aptamer, broccoli aptamer, mango aptamer, corn aptamer, or variant thereof, e.g. baby spinach or spinach 2 aptamers, capable of comprising a detectable label.

In various embodiments, one or more of the aptamers 1402, 1404, 1406 can comprise an aptamer barcode sequence. In some embodiments, an aptamer barcode sequence can be located adjacent to the 3′ end of the at least one aptamer. In some embodiments, an aptamer barcode sequence can be located adjacent to the 5′ end of the at least one aptamer. In some embodiments, an aptamer barcode sequence can be located within 50 nucleotides of either the 3′ end or the 5′ end of the at least one aptamer. In some embodiments, an aptamer barcode sequence can be located at a position on the at least one aptamer that does not comprise a secondary structure.

In various embodiments, a hybridization sequence 1412 of the first aptamer 1402 comprises a first aptamer barcode sequence and the hybridization sequence 1422 of the second aptamer 1406 comprises a second aptamer barcode sequence, wherein the first aptamer barcode sequence can be a reverse complement of the second aptamer barcode sequence. In various embodiments, hybridization of the first aptamer barcode sequence to the second aptamer barcode sequence can be detectable and indicates correct aptamer pairing.

In various embodiments a second hybridization sequence 1424 of the second aptamer 1406 comprises a first aptamer barcode sequence and the hybridization sequence 1436 of the third aptamer 1406 comprises a second aptamer barcode sequence, wherein the first aptamer barcode sequence can be a reverse complement to the second aptamer barcode sequence. In various embodiments a hybridization of the first aptamer barcode sequence to the second aptamer barcode sequence can be detectable and indicates correct aptamer pairing.

In various embodiments, aptamer barcode sequences can be configured to indicate internalization of the one or more aptamers into a cell.

In various embodiments, a hybridization sequence 1412 of the first aptamer 1402 can be positioned between the cell surface binding domain 1416 and the 3′ end 1408 of the first aptamer 1402.

In various embodiments, a hybridization sequence 1412 of the first aptamer 1402 can be positioned between the cell surface binding domain 1416 and the 5′ 1410 end of the first aptamer 1402.

In various embodiments, a hybridization sequence 1422, 1424 of the second aptamer can be positioned between the payload binding domain 1426 and the 3′ 1418 end of the second aptamer 1404.

In various embodiments, a hybridization sequence 1422, 1424 of the second aptamer 1404 can be positioned between the payload binding domain 1426 and the 5′ 1420 end of the second aptamer 1404.

In various embodiments, a hybridization sequence 1436 of the third aptamer 1406 is positioned between the payload binding domain 1434 and the 3′ 1430 end of the third aptamer 1406.

In various embodiments, a hybridization sequence 1436 of the third aptamer 1406 is positioned between the payload binding domain 1434 and the 5′ 1432 end of the third aptamer 1406.

In various embodiments, hybridization sequences 1412, 1422, 1424, 1436 can range in length from 12-20 nucleotides. In various embodiments, hybridization sequences 1412, 1422, 1424, 1436 can range in length from 8-30 nucleotides.

In various embodiments, a cell surface molecule 1444 can be bound to a cell surface binding domain 1416. In various embodiments, a cell surface molecule 1444 can be part of the internalization complex 1400. In some embodiments, cell surface molecule 1326 is a cell surface protein, e.g., an endogenous cell surface protein that can be internalized, e.g., by endocytosis. In some embodiments, the binding of the internalization complex to the cell surface molecule induces internalization of the cell surface molecule with the internalization complex bound thereto. In various embodiments, a cell surface molecule 1444 can comprise a receptor tyrosine kinase, e.g., PTK7, Axl, PDGFRb, IR, or EGFR. In various embodiments, a cell surface molecule 1444 can comprise a transmembrane protein, e.g., IL-6R, EpCAM or PSMA.

In various embodiments, at least one of the payload binding domains 1426, 1434 further comprises a payload handle. In some embodiments, a payload handle comprises a protein recognition sequence. In some embodiments, a payload handle comprises a biotinylated structure. In some embodiments, a payload handle comprises a poly-A tail capture sequence. In some embodiments, a payload can be bound to at least one the payload binding domains 1426, 1434.

In various embodiments, a payload binding domain (e.g., can comprise a payload handle. In various embodiments, a payload handle can act as a universal adaptor allowing one or more standard or known aptamer configurations to be used to bind as many different types and variants of payloads as necessary for internalization.

In various embodiments, a protein recognition sequence can be used as a payload handle. In various embodiments, a protein recognition sequence can be common throughout proteins of a specified class or type. In various embodiments, a protein recognition sequence can be designed to recognize a group of proteins of interest. In various embodiments, a protein recognition sequence of an aptamer can be designed to bind to a protein sequence or higher order structure inherent to a payload (e.g., 1438, 1440). In various embodiments, a protein recognition sequence of an aptamer can be designed to bind to a protein sequence or higher order structure that has been artificially generated or added to a payload.

In various embodiments, a payload binding domain (e.g., 1426, 1434) can comprise a first payload handle including biotin and a payload (e.g., 1438, 1440) can comprise a second payload handle including a biotin-binding protein (e.g. avidin or streptavidin). In various non-limiting examples, the payload (e.g., 1438, 1440) can be any molecule capable of binding the biotin-binding protein (e.g. several examples are described herein).

In various embodiments, a payload binding domain (e.g., 1426, 1434) can comprise a first payload handle including a biotin-binding protein (e.g. avidin or streptavidin) and a payload (e.g., 1438, 1440) can comprise a second payload handle including biotin. In various non-limiting examples, the payload (e.g., 1438, 1440) can be any molecule capable of binding to biotin (e.g. several examples are described herein).

In various embodiments, a payload (e.g., 1438, 1440) can comprise a payload handle. In various embodiments, a payload binding domain (e.g., 1426, 1434) can comprise a payload handle. In various embodiments, both a payload (e.g., 1438, 1440) and a payload binding domain (e.g., 1426, 1434) can comprise payload handles that can be configured to interact with one another and act as universal adaptors. A skilled artisan will appreciate that a variety of payload handle combinations and permutations can be utilized for general and specific purposes.

In various embodiments, a payload handle can be covalently bound to a payload binding domain (e.g., 1426, 1434). In various embodiments, a payload handle can be non-covalently bound to a payload binding domain (e.g., 1426, 1434).

In various embodiments, a payload handle can be covalently bound to a payload (e.g., 1438, 1440). In various embodiments, a payload handle can be non-covalently bound to a payload (e.g., 1438, 1440).

In some embodiments, a payload 1438, 1440 comprises a Cas molecule. In some embodiments, a payload 1438, 1440 comprises a gRNA molecule. In some embodiments, a 1438, 1440 payload comprises an RNP complex, wherein the RNP complex comprises a Cas molecule associated with a gRNA molecule. In some embodiments, a payload 1438, 1440 comprises an mRNA molecule. In some embodiments, a payload 1438, 1440 comprises an oligonucleotide. In some embodiments, a payload 1438, 1440 comprises a protein. In some embodiments, a payload 1438, 1440 comprises an RNAi molecule.

In some embodiments, a cell surface binding domain 1416 comprises a secondary structure. In some embodiments, a secondary structure of the cell surface binding domain 1416 comprises a stem loop. In some embodiments, a cell surface binding domain 1444 comprises a tertiary structure. Aptamers having cell surface binding domains, and design of aptamers having cell surface binding domains, have been described herein.

In some embodiments, a payload binding domain 1426, 1434 comprises a secondary structure. In some embodiments, a payload binding domain 1426, 1434 comprises a secondary structure of the payload binding domain comprises a stem loop. In some embodiments, a payload binding domain 1426, 1434 comprises a tertiary structure. In some embodiments, a payload 1438, 1440 can be bound to each of the payload binding domains 1426, 1434. In some embodiments, a payload 1438 of the second aptamer 1404 can be different from a payload 1440 of the third aptamer 1406. In some embodiments, a payload 1438 of the second aptamer 1404 can be the same as the payload 1440 of the third aptamer 1406.

In some embodiments, a sequence similarity between the hybridization sequence 1412 of the first aptamer 1402 and the second hybridization sequence 1424 of the second aptamer 1404 is less than 25%. In some embodiments, a sequence similarity between the hybridization sequence 1412 of the first aptamer 1402 and the hybridization sequence 1436 of the third aptamer 1406 is less than 25%. In some embodiments, a sequence similarity between the hybridization sequence 1436 of the third aptamer and the first hybridization sequence 1422 of the second aptamer 1404 is less than 25%.

In various embodiments, a sequence similarity between the hybridization sequence 1412 of the first aptamer 1402 and the second hybridization sequence 1424 of the second aptamer 1404 is less than 20%. In various embodiments, a sequence similarity between the hybridization sequence 1412 of the first aptamer 1402 and the hybridization sequence 1436 of the third aptamer 1406 is less than 20%. In various embodiments, a sequence similarity between the hybridization sequence 1436 of the third aptamer 1406 and the first hybridization sequence 1422 of the second aptamer 1404 is less than 20%.

In various embodiments, a sequence similarity between the hybridization sequence 1412 of the first aptamer 1402 and the second hybridization sequence 1424 of the second aptamer 1404 comprises fewer than 8 consecutive complementary bases. In various embodiments, a sequence similarity between the hybridization sequence 1412 of the first aptamer 1402 and the hybridization sequence 1436 of the third aptamer 1406 comprises fewer than 8 consecutive complementary bases. In various embodiments, a sequence similarity between the hybridization sequence 1436 of the third aptamer 1406 and the first hybridization sequence 1422 of the second aptamer 1404 comprises fewer than 8 consecutive complementary bases.

In various embodiments, a hybridization sequence 1412 of the first aptamer and the first hybridization sequence 1422 of the second aptamer 1404 have a sequence complementarity that ranges from 75% to 100%.

In various embodiments, a hybridization sequence 1436 of the third aptamer 1406 and the second hybridization sequence 1424 of the second aptamer 1404 have a sequence complementarity that ranges from 75% to 100%.

In various embodiments, a composition 1400 can comprise a bead and the bead can comprise an oligonucleotide. In various embodiments, an oligonucleotide can comprise a nucleic acid barcode molecule comprising a bead specific barcode. In various embodiments, an oligonucleotide can comprise a capture sequence and the capture sequence can be complementary to at least a portion of at least one aptamer 1402, 1404, 1406. In some embodiments, an oligonucleotide can further comprise a unique molecular identifier (UMI).

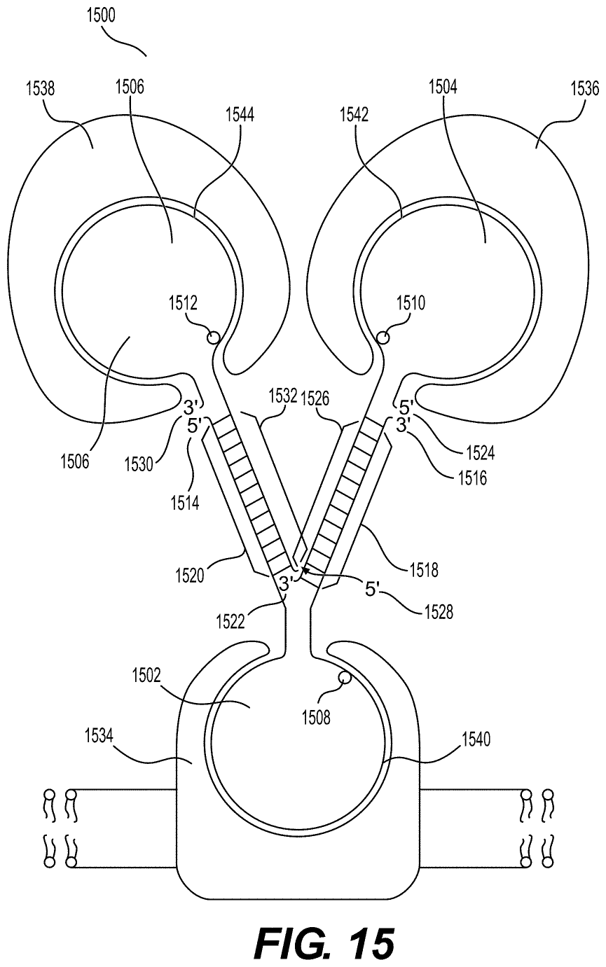

FIG. 15 is an illustration depicting an internalization complex 1500 comprising three aptamers in a branched format with two payloads each bound to an aptamer and a cell surface molecule bound to another aptamer according to various embodiments.

In various embodiments, a composition for internalizing a plurality of payloads into a cell is provided. In a non-limiting example, three aptamers can be hybridized to one another to generate a branched format. In some embodiments, a first aptamer 1502 can be hybridized to a second aptamer 1504 and a third aptamer 1506.

In various embodiments, a first aptamer 1502 can comprise a 3′ end 1516 and a 5′ end 1514. In various embodiments, a first aptamer 1502 can comprise a cell surface binding domain 1540 configured to bind with a cell surface molecule 1534. In various embodiments, a first aptamer 1502 can comprise a detectable label 1508. In some embodiments, a first aptamer 1502 can comprise a first aptamer hybridization sequence 1518 that can hybridize with a hybridization sequence 1526 of a second aptamer 1504. In some embodiments, a first aptamer 1502 can comprise a second aptamer hybridization sequence 1520 that can hybridize with a hybridization sequence 1532 of a third aptamer 1506.

In various embodiments of the branched aptamer format, a second aptamer 1504 can comprise a 3′ end 1522 and a 5′ end 1524. In various embodiments, a second aptamer 1504 can comprise a payload binding domain 1542 configured to bind a payload 1536. In some embodiments, a second aptamer 1504 can comprise a detectable label 1510.

In various embodiments, a third aptamer 1506 can comprise a 3′ end 1530 and a 5′ end 1528. In some embodiments, a third aptamer 1506 can comprise a payload binding domain 1544 configured to bind a payload 1538. In various embodiments, a third aptamer 1506 can comprise a detectable label 1512.

In various embodiments, the detectable labels 1508, 1510, 1512 can comprise a fluorescent molecule. In some embodiments, the detectable labels 1508, 1510, 1512 can comprise a quenching agent configured to interact with the fluorescent molecule and decrease fluorescence. In some embodiments, the detectable labels 1508, 1510, 1512 can be activated upon internalization of at least one of the aptamers 1502, 1504, 1506 into a cell. In some embodiments, the detectable labels 1508, 1510, 1512 can be activated upon binding a payload to at least one of the aptamers 1502, 1504, 1506. In some embodiments, a detectable label 1508, 1510, 1512 can be activated upon binding a cell surface molecule 1534 to at least one of the aptamers. As discussed earlier herein, aptamers, e.g., first and/or second and/or third aptamers, may comprise the detectable label due to inclusion of a domain (or variant of a domain) of an aptamer such as a spinach aptamer, broccoli aptamer, mango aptamer, corn aptamer, or variant thereof, e.g. baby spinach or spinach 2 aptamers, capable of comprising a detectable label.

In various embodiments, one or more of the aptamers 1502, 1504, 1506 can comprise an aptamer barcode sequence. In some embodiments, an aptamer barcode sequence can be located adjacent to the 3′ 1522, 1516, 1530 end of the at least one aptamer 1502, 1504, 1506. In some embodiments, an aptamer barcode sequence can be located adjacent to the 5′ 1514, 1524, 1528 end of the at least one aptamer. In some embodiments, an aptamer barcode sequence can be located within 50 nucleotides of either the 3′ end 1522, 1516, 1530 or the 5′ end 1514, 1524, 1528 of the at least one aptamer 1502, 1504, 1506. In some embodiments, an aptamer barcode sequence can be located at a position on an aptamer 1502, 1504, 1506 that does not comprise a secondary structure.

In various embodiments, a first hybridization sequence 1518 of the first aptamer 1502 comprises a first aptamer barcode sequence and a hybridization sequence 1526 of the second aptamer 1504 comprises a second aptamer barcode sequence. In some embodiments, a first aptamer barcode sequence can be a reverse complement to a second aptamer barcode sequence. In some embodiments, a first aptamer barcode sequence hybridized to a second aptamer barcode sequence can be detectable and indicates correct aptamer pairing.

In various embodiments, a second hybridization sequence 1520 of a first aptamer 1502 comprises a first aptamer barcode sequence and a hybridization sequence 1532 of a third aptamer 1506 comprises a second aptamer barcode sequence. In some embodiments, a first aptamer barcode sequence can be a reverse complement to a second aptamer barcode sequence. In some embodiments, a first aptamer barcode sequence hybridized to a second aptamer barcode sequence can be detectable and indicates correct aptamer pairing.

In various embodiments, aptamer barcode sequences can be configured to indicate internalization of the one or more aptamers 1502, 1504, 1506 into a cell.

In various embodiments, a hybridization sequence 1518, 1520 of the first aptamer 1502 can be positioned between the cell surface binding domain 1540 and the 3′ end 1516 of the first aptamer 1502.

In various embodiments, a hybridization sequence 1518, 1520 of the first aptamer 1502 can be positioned between the cell surface binding domain 1540 and the 5′ 1514 end of the first aptamer 1502.

In various embodiments, a hybridization sequence 1526 of the second aptamer 1504 can be positioned between the payload binding domain 1542 and the 3′ 1522 end of the second aptamer 1504.

In various embodiments, a hybridization sequence 1526 of the second aptamer 1504 can be positioned between the payload binding domain 1542 and the 5′ 1524 end of the second aptamer 1504.

In various embodiments, a hybridization sequence 1532 of the third aptamer 1506 can be positioned between the payload binding domain 1544 and the 3′ 1530 end of the third aptamer 1506.

In various embodiments, a hybridization sequence 1532 of the third aptamer 1506 can be positioned between the payload binding domain 1544 and the 5′ 1528 end of the third aptamer 1506.

In various embodiments, hybridization sequences 1518, 1520, 1526, 1532 can range in length from 12-20 nucleotides. In various embodiments, hybridization sequences 1518, 1520, 1526, 1532 can range in length from 8-30 nucleotides.

In various embodiments, a cell surface molecule 1534 can be bound to a cell surface binding domain 1540. In some embodiments, a cell surface molecule 1534 is part of an internalization complex 1500. In some embodiments, a cell surface molecule 1534 comprises a receptor tyrosine kinase. In some embodiments, a cell surface molecule 1534 comprises a transmembrane protein.

In various embodiments, at least one of the payload binding domains 1542, 1544 further comprises a payload handle. In some embodiments, a payload handle comprises a protein recognition sequence. In some embodiments, a payload handle comprises a biotinylated structure. In some embodiments, a payload handle comprises a poly-A tail capture sequence. In some embodiments, a payload 1536, 1538 can be bound to at least one the payload binding domains 1542, 1544.

In some embodiments, a payload 1536, 1538 comprises a Cas molecule. In some embodiments, a payload 1536, 1538 comprises a gRNA molecule. In some embodiments, a 1536, 1538 payload comprises an RNP complex, wherein the RNP complex comprises a Cas molecule associated with a gRNA molecule. In some embodiments, a payload 1536, 1538 comprises an mRNA molecule. In some embodiments, a payload 1536, 1538 comprises an oligonucleotide. In some embodiments, a payload 1536, 1538 comprises a protein. In some embodiments, a payload 1536, 1538 comprises an RNAi molecule.

In some embodiments, a cell surface binding domain 1540 comprises a secondary structure. In some embodiments, a secondary structure of the cell surface binding domain 1540 comprises a stem loop. In some embodiments, a cell surface binding domain 1540 comprises a tertiary structure. Aptamers having a cell surface binding domain, and design of aptamers having xa cell surface binding domain, have been described herein.

In some embodiments, a payload binding domain 1542, 1544 comprises a secondary structure. In some embodiments, a payload binding domain 1542, 1544 comprises a secondary structure of the payload binding domain comprises a stem loop. In some embodiments, a payload binding domain 1542, 1544 comprises a tertiary structure. In some embodiments, a payload 1536, 1538 can be bound to each of the payload binding domains 1542, 1544. In some embodiments, a payload 1536 of the second aptamer 1504 can be different from a payload 1538 of the third aptamer 1506. In some embodiments, a payload 1536 of the second aptamer 1504 can be the same as the payload 1538 of the third aptamer 1506.

In various embodiments, a sequence similarity between a second hybridization sequence 1520 of the first aptamer 1502 and a hybridization sequence 1526 of the second aptamer 1504 can be less than 25%. In various embodiments, a sequence similarity between a first hybridization sequence 1518 of a first aptamer 1502 and a hybridization sequence 1532 of the third aptamer 1506 can be less than 25%. In various embodiments, a sequence similarity between a hybridization sequence 1532 of a third aptamer 1506 and a hybridization sequence 1526 of a second aptamer 1504 can be less than 25%.

In various embodiments, a sequence similarity between a second hybridization sequence 1520 of the first aptamer 1502 and a hybridization sequence 1526 of the second aptamer 1504 can be less than 20%. In various embodiments, a sequence similarity between a first hybridization sequence 1518 of a first aptamer 1502 and a hybridization sequence 1532 of the third aptamer 1506 can be less than 20%. In various embodiments, a sequence similarity between a hybridization sequence 1532 of a third aptamer 1506 and a hybridization sequence 1526 of a second aptamer 1504 can be less than 20%.

In various embodiments, a sequence similarity between a second hybridization sequence 1520 of the first aptamer 1502 and a hybridization sequence 1526 of the second aptamer 1504 comprises fewer than 8 consecutive complementary bases. In various embodiments, a sequence similarity between a first hybridization sequence 1518 of the first aptamer 1502 and a hybridization sequence 1532 of the third aptamer 1506 comprises fewer than 8 consecutive complementary bases. In various embodiments, a sequence similarity between a hybridization sequence 1532 of the third aptamer 1506 and a hybridization sequence 1526 of a second aptamer 1504 comprises fewer than 8 consecutive complementary bases.

In various embodiments, a first hybridization sequence 1518 of a first aptamer 1502 and a hybridization sequence 1526 of a second aptamer 1504 have a sequence complementarity that ranges from 75% to 100%. In various embodiments, a second hybridization sequence 1520 of a first aptamer 1502 and a hybridization sequence 1532 of a third aptamer 1506 have a sequence complementarity that ranges from 75% to 100%.

In various embodiments, a composition 1500 can comprise a bead and the bead can comprise an oligonucleotide. In various embodiments, an oligonucleotide can comprise a nucleic acid barcode molecule comprising a bead specific barcode. In various embodiments, an oligonucleotide can comprise a capture sequence and the capture sequence can be complementary to at least a portion of at least one aptamer 1502, 1504, 1506. In some embodiments, an oligonucleotide can further comprise a unique molecular identifier (UMI).

### Multiple Aptamer Internalization Complexes—Various Configurations

Aspects of the disclosure comprise systems including multiple different (or the same) compositions having one or more aptamers hybridized to one another. Such embodiments can allow internalization complexes to carry multiple payloads capable of being internalized in a single internalization event. Such embodiments can be particularly useful when multiple payloads interact with one another in a biological process. For a non-limiting example, many cell signaling pathways involve multiple molecules that can be delivered by multi-aptamer internalization complexes. For another non-limiting example, many gene regulatory elements can be required to up or down regulate one or more genes and the related elements can be delivered via the systems disclosed herein. Skilled artisans will appreciate the versatility of the multi-aptamer internalization complexes described here as there are countless applications.

Additional embodiments can comprise systems including any of the two or three aptamers internalization complexes described herein to achieve the purposes described above. Other embodiments may comprise systems comprising 4, 5, 6, 7, 8, 9, 10 any number of internalization complexes.

In various embodiments, systems for internalizing one or more payload molecules into a cell can comprise a plurality of internalization complexes comprising a first aptamer and a second aptamer. In some embodiments, systems for internalizing a variety of different payload molecules can comprise a plurality of internalization complexes comprising a first aptamer and a second aptamer and a plurality internalization complexes comprising a first aptamer, a second aptamer, and a third aptamer. In some embodiments, the internalization complexes may vary greatly and bind a variety of different payloads with their payload binding domains. In some embodiments, internalization complexes may vary widely in their affinity for the same or different cell surface molecules. A skilled artisan will appreciate that almost an infinite number of permutations systems can offer and those systems can be designed to achieve any number of purposes. In some embodiments, the purposes may be experimental in nature (e.g. used to understand biological systems). In some embodiments, the purposes can be therapeutic (e.g. used to delivery cell therapy molecules). In some embodiments, the purposes can be for gene editing (e.g. delivery of CRISPR related molecules).

In embodiments comprising elaborate systems comprising many different internalization complexes, design of aptamer hybridization sequences can be done in such a way as to ensure that aptamers correctly hybridize to one another. In systems involving two-aptamer complexes, design of hybridization sequences for the aptamers can be less complicated because there can be a potential for less cross-talk between aptamers. In some embodiments involving three or more aptamers, aptamer hybridization sequences can be carefully designed such that the correct aptamers couple to one another through hybridization. In some embodiments, hybridization sequences of the various aptamers can be selected to ensure correct hybridization.

In various systems, aptamers can comprise labels such as those described herein, via the example domains (or variants thereof) described herein, and be used in the same ways described (e.g. in previous sections). In various embodiments, labels can be designed to differentiate between different aptamers in systems involving a multitude of aptamers.

In various systems, aptamers can comprise aptamer barcode sequences. Aptamer barcode sequences can be used as described in previous sections here. In embodiments involving a plurality of internalization complexes, aptamer barcodes can be especially useful. In some embodiments, aptamer barcode sequences can be sequenced. In some embodiments, sequencing can be done after internalization has occurred.

A non-limiting example for use of an aptamer barcode sequence can comprise a hybridization sequence of at least one of the aptamers described herein comprising a first aptamer barcode sequence and specifically targets a hybridization sequence of at least another aptamer comprising a second aptamer barcode sequence. In some embodiments, a first aptamer barcode sequence can be a reverse complement to a second aptamer barcode sequence. In some embodiments, a first aptamer barcode sequence hybridized to a second aptamer barcode sequence is detectable and indicates correct aptamer pairing. In some embodiments, aptamer barcode sequences can be configured to indicate internalization of the one or more aptamers into a cell.

In various embodiments, systems for internalizing a plurality of payloads can comprise sets of aptamer complexes (e.g. internalization complexes). In some embodiments, sets of aptamer complexes can comprise two-aptamer internalization complexes. In some embodiments, sets of aptamer complexes can comprises three-aptamer internalization complexes having a non-branched configuration. In some embodiments, sets of aptamer complexes can comprises three-aptamer internalization complexes having a branched configuration.

In some embodiments, different aptamer complexes (e.g. one or more aptamers hybridized to one another) can be generated and then pooled together to prevent incorrect aptamer hybridization. In alternative embodiments, aptamers can be pooled together and then hybridized into different aptamer complexes.

### III. Methods

Aspects of the disclosure comprise methods of use for internalization of a payload(s) into a cell. In various embodiments, the methods described herein can be applied to any of the compositions and systems described throughout this application.

Methods for Internalizing a Payload into a Cell

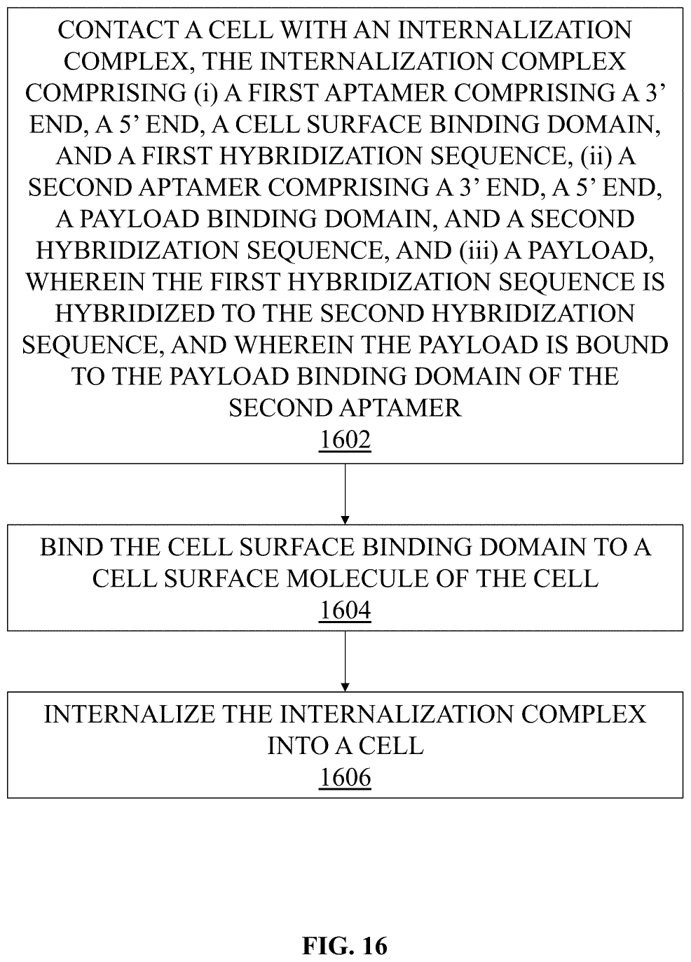

Referring to FIG. 16, aspects of the disclosure comprise methods for internalizing a payload into a cell. In various embodiments, a method for internalization of a payload into a cell can comprise contacting a cell with an internalization complex, the internalization complex comprising (i) a first aptamer comprising a 3′ end, a 5′ end, a cell surface binding domain, and a first hybridization sequence, (ii) a second aptamer comprising a 3′ end, a 5′ end, a payload binding domain, and a second hybridization sequence, and (iii) a payload, wherein the first hybridization sequence is hybridized to the second hybridization sequence, and wherein the payload is bound to the payload binding domain of the second aptamer 1602.

In various embodiments, a method for internalization of a payload into a cell can comprise binding the cell surface binding domain to a cell surface molecule of the cell 1604.

In various embodiments, a method for internalization of a payload into a cell can comprise internalize the internalization complex into a cell 1606.

In various embodiments, a method for internalization of a payload into a cell can further comprise contacting a cell with an internalization complex. In some embodiments, a method for internalization of a payload into a cell can comprise binding a cell surface binding domain to a cell surface molecule. In some embodiments, a method for internalization of a payload into a cell can comprise internalizing an internalization complex into a cell.

In various embodiments, a method for internalization of a payload into a cell can comprise activating a detectable label upon binding a payload binding domain to a payload.

In various embodiments, a method for internalization of a payload into a cell can comprise activating a detectable label upon binding a cell surface binding domain to a cell surface molecule.

In various embodiments, a method for internalization of a payload into a cell can comprise activating a detectable label upon internalization of an internalization complex.

In some embodiments, a first aptamer comprises a detectable label. In some embodiments, a second aptamer comprises a detectable label. The first and/or second aptamer may comprise the detectable label via inclusion of a domain (or variant of a domain) of an aptamer such as a spinach aptamer, broccoli aptamer, mango aptamer, corn aptamer, or variant thereof, e.g. baby spinach or spinach 2 aptamers, capable of binding a detectable label.

In various embodiments, a method for internalization of a payload into a cell can comprise emitting a light signal from a detectable label using a fluorescent molecule. In some embodiments, quenching an emitted light signal from a detectable label can be accomplished by interacting a quenching agent with the fluorescent molecule. In some embodiments, a fluorescent molecule can be quenched prior to activating. In some embodiments, a fluorescent molecule can be quenched after activating.

In various embodiments, at least one of the first aptamer and the second aptamer comprises an aptamer barcode sequence. In various embodiments, a method for internalization of a payload into a cell can comprise confirming internalization of an internalization complex. In some embodiments, a confirming step can use at least one aptamer barcode sequence. In some embodiments, at least one aptamer is detectable. In some embodiments, an aptamer barcode sequence can be located on at least one of the aptamers. In some embodiments, a method can comprise sequencing at least one aptamer barcode sequence.

In various embodiments, aptamers can be sequenced in their entireties. In some embodiments, aptamers can be partially sequenced. Aspects of the disclose can comprise a variety of different sequencing methods. Some non-limiting examples of sequence methods can comprise rolling circle sequencing or using SMRT™ systems produced by PacBio™.

In various embodiments, a method for internalization of a payload into a cell can comprise confirming correct aptamer pairing. In some embodiments, the step of confirming correct aptamer pairing comprises a fluorescence in situ hybridization method.

In various embodiments, a method for internalization of a payload into a cell can comprise binding a capture sequence to one of the aptamers, wherein the capture sequence can be complementary to at least a portion of an aptamer.

In various embodiments, a method can comprise partitioning the cell into a partition with a plurality of nucleic acid barcode molecules, wherein a nucleic acid barcode molecule of the plurality of nucleic acid barcode molecules comprises a partition-specific barcode sequence and a capture sequence that can be complementary to at least a portion of one of the aptamers. In various embodiments, the method can comprise hybridizing the capture sequence of the nucleic acid barcode molecule to the at least a portion of the one of the aptamers. In various embodiments, the method can comprise using the nucleic acid barcode molecule and the one of the aptamers to generate a barcoded product comprising the partition-specific barcode sequence or a complement thereof and the aptamer barcode sequence or a complement thereof.

In various embodiments, the method can comprise partitioning the cell and a bead into the partition, wherein the bead comprises the plurality of nucleic acid barcode molecules. In some embodiments, the nucleic acid barcode molecule of the plurality of nucleic acid barcode molecules further comprises a unique molecular identifier (UMI).

In various embodiments, the method can comprise determining the sequence of the barcoded product or a portion thereof. In some embodiments, the method can comprise confirming internalization of the internalization complex in the cell if the sequence of the barcoded product or portion thereof contains (i) the partition-specific barcode sequence or complement thereof and (ii) the aptamer barcode sequence or complement thereof.

In some embodiments, the method comprises generating one or more of the aptamers with a polynucleotide synthesis procedure.

In various embodiments, a method of preparing an internalization complex for internalization of a payload into a cell can comprise hybridizing a hybridization sequence of a first aptamer to a hybridization sequence of a second aptamer, wherein the first aptamer comprises a 3′ end, a 5′ end, a cell surface binding domain, and the hybridization sequence of the first aptamer, wherein the second aptamer comprises a 3′ end, a 5′ end, a payload binding domain, and the hybridization sequence of the second aptamer.

Methods for Internalizing Multiple Payloads into a Cell

In various embodiments, a method for internalization of a payload into a cell can comprise generating one or more of the aptamers with a polynucleotide synthesis procedure.

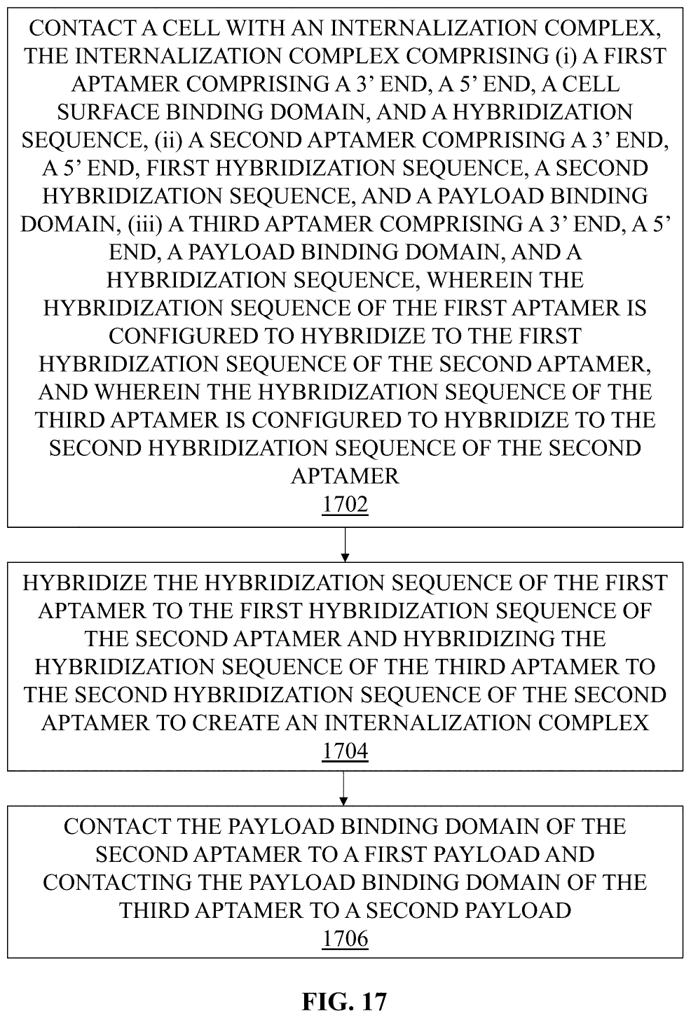

Referring to FIG. 17, aspects of the disclosure include methods for internalization of a plurality of payloads into a cell. In various embodiments, methods include use of the compositions of systems described herein. In some embodiments, methods include use of one or more two-aptamer internalization complexes. In some embodiments, methods can comprise one or more three-aptamer internalization complexes. In some embodiments, methods can comprise aptamer complexes in a non-branched format. In some embodiments, methods can comprise aptamer complexes in a branched format.

In various embodiments, a method for internalization of a plurality of payloads into a cell can comprise contacting a cell with an internalization complex, the internalization complex comprising (i) a first aptamer comprising a 3′ end, a 5′ end, a cell surface binding domain, and a hybridization sequence, (ii) a second aptamer comprising a 3′ end, a 5′ end, first hybridization sequence, a second hybridization sequence, and a payload binding domain, (iii) a third aptamer comprising a 3′ end, a 5′ end, a payload binding domain, and a hybridization sequence, wherein the hybridization sequence of the first aptamer is configured to hybridize to the first hybridization sequence of the second aptamer, and wherein the hybridization sequence of the third aptamer is configured to hybridize to the second hybridization sequence of the second aptamer 1702.

In various embodiments, a method for internalization of a plurality of payloads into a cell can comprise hybridizing the hybridization sequence of the first aptamer to the first hybridization sequence of the second aptamer and hybridizing the hybridization sequence of the third aptamer to the second hybridization sequence of the second aptamer to create an internalization complex 1704.

In various embodiments, a method for internalization of a plurality of payloads into a cell can comprise contacting the payload binding domain of the second aptamer to a first payload and contacting the payload binding domain of the third aptamer to a second payload 1706.

In various embodiments, a method for internalization of a plurality of payloads into a cell can comprise contacting a cell with an internalization complex. In some embodiments, the method can comprise binding a cell surface binding domain to a cell surface molecule. In some embodiments, the method can comprise internalizing internalization complexes.

In various embodiments, a method for internalization of a plurality of payloads into a cell can comprise activating a detectable label upon binding a payload binding domain a payload.

In various embodiments, a method for internalization of a plurality of payloads into a cell can comprise activating a detectable label upon binding the cell surface binding domain to the cell surface molecule.

In various embodiments, a method for internalization of a plurality of payloads into a cell can comprise activating a detectable label upon internalization of an internalization complex.

In some embodiments, a first aptamer comprises a detectable label. In some embodiments, a second aptamer comprises the detectable label. In some embodiments, a third aptamer comprises the detectable label. The first and/or second and/or third aptamer may comprise the detectable label via inclusion of domain as discussed earlier herein.

In various embodiments, detectable labels can be used to label any of the aptamers described herein. In some embodiments, detectable labels can be used to identify aptamers. In some embodiments comprising methods using a variety of different aptamers, labels can be especially useful in identifying aptamers and which aptamers have been internalized into a cell.

In various embodiments, a method for internalization of a plurality of payloads into a cell can comprise emitting a light signal from a detectable label using a fluorescent molecule. In various embodiments, a method for internalization of a plurality of payloads into a cell can comprise quenching an emitted light signal from the detectable label by interacting a quenching agent with a fluorescent molecule. In some embodiments, a fluorescent molecule can be quenched prior to activating. In some embodiments, a fluorescent molecule can be quenched after activating.

In various embodiments, at least one of the first aptamer and the second aptamer comprises an aptamer barcode sequence. In various embodiments, a method for internalization of a plurality of payloads into a cell can comprise confirming internalization of an internalization complex. In some embodiments, a confirming step can use at least one aptamer barcode sequence. In some embodiments, at least one aptamer is detectable. In some embodiments, an aptamer barcode sequence can be located on at least one aptamer. In some embodiments, the method can comprise sequencing the at least one aptamer barcode sequences. In some embodiments, the method can comprise confirming correct aptamer pairing. In some embodiments, confirming correct aptamer pairing comprises a fluorescence in situ hybridization method.

In various embodiments, a method for internalization of a plurality of payloads into a cell can comprise binding a capture sequence to at least one of the aptamers. In some embodiments, a capture sequence can be complementary to a portion of at least one aptamer.

In various embodiments, a method for internalization of a plurality of payloads into a cell can comprise a bead comprising an oligonucleotide. In some embodiments, an oligonucleotide can comprise a nucleic acid barcode molecule comprising a bead specific barcode. In some embodiments, an oligonucleotide can comprise a capture sequence. In some embodiments, an oligonucleotide can comprise a unique molecular identifier (UMI).

In some embodiments, an oligonucleotide can comprise generating one or more of the aptamers with a polynucleotide synthesis procedure.

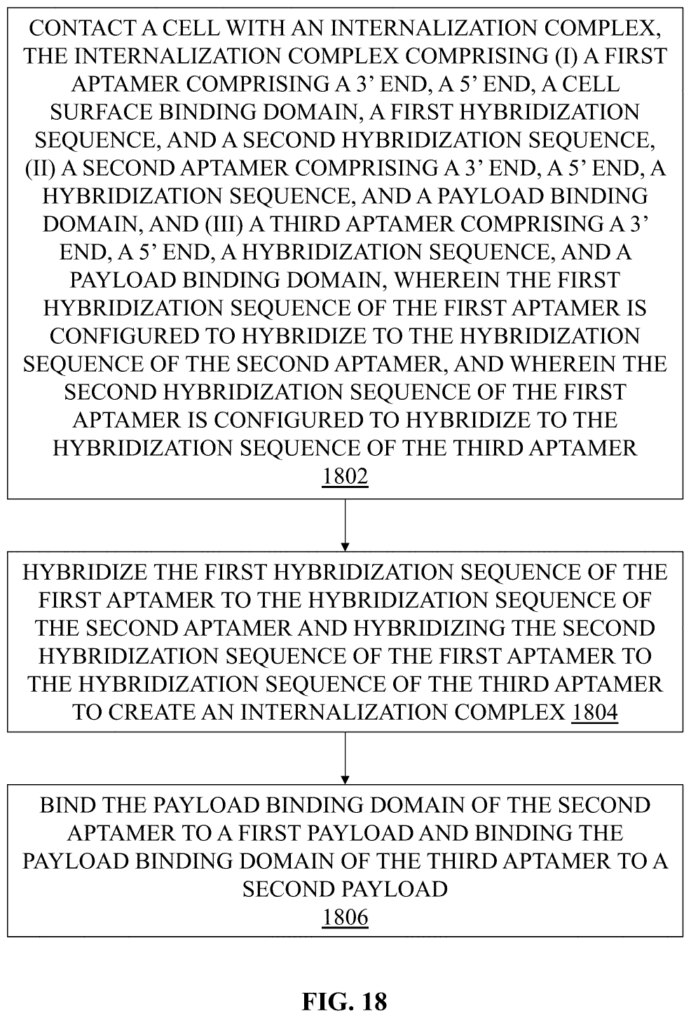

Referring to FIG. 18, aspects of the disclosure can comprise a method for internalization of a plurality of payloads into a cell wherein internalization complexes can comprise branched configurations. In various embodiments, a method for internalization of a plurality of payloads into a cell can comprise contacting a cell with an internalization complex, the internalization complex comprising (i) a first aptamer comprising a 3′ end, a 5′ end, a cell surface binding domain, a first hybridization sequence, and a second hybridization sequence, (ii) a second aptamer comprising a 3′ end, a 5′ end, a hybridization sequence, and a payload binding domain, and (iii) a third aptamer comprising a 3′ end, a 5′ end, a hybridization sequence, and a payload binding domain, wherein the first hybridization sequence of the first aptamer is configured to hybridize to the hybridization sequence of the second aptamer, and wherein the second hybridization sequence of the first aptamer is configured to hybridize to the hybridization sequence of the third aptamer 1802.

In various embodiments, a method for internalization of a plurality of payloads into a cell can comprise hybridizing the first hybridization sequence of the first aptamer to the hybridization sequence of the second aptamer and hybridizing the second hybridization sequence of the first aptamer to the hybridization sequence of the third aptamer to create an internalization complex 1804.

In various embodiments, a method for internalization of a plurality of payloads into a cell can comprise binding the payload binding domain of the second aptamer to a first payload and binding the payload binding domain of the third aptamer to a second payload 1806.

In various embodiments, a method for internalization of a plurality of payloads into a cell can comprise contacting a cell with an internalization complex. In some embodiments, methods can comprise, binding a cell surface binding domain to a cell surface molecule. In some embodiments, methods can comprise internalizing an internalization complex.

In various embodiments, a method for internalization of a plurality of payloads into a cell can comprise activating a detectable label upon binding one or more payload binding domains to one or more of the payloads.

In various embodiments, a method for internalization of a plurality of payloads into a cell can comprise activating a detectable label upon binding a cell surface binding domain to a cell surface molecule.

In various embodiments, a method for internalization of a plurality of payloads into a cell can comprise activating a detectable label upon internalization of the internalization complex.

In some embodiments, a first aptamer comprises the detectable label. In some embodiments, a second aptamer comprises the detectable label. In some embodiments, a third aptamer comprises the detectable label. In embodiments, the first and/or second and/or third aptamer may comprise the detectable by inclusion of a domain as discussed earlier herein.

In various embodiments, a method for internalization of a plurality of payloads into a cell can comprise emitting a light signal from the detectable label using a fluorescent molecule.

In various embodiments, a method for internalization of a plurality of payloads into a cell can comprise quenching an emitted light signal from a detectable label by interacting a quenching agent with a fluorescent molecule. In some embodiments, a fluorescent molecule can be quenched prior to activating. In some embodiments, a fluorescent molecule can be quenched after activating.

In various embodiments, at least one of the first aptamer and the second aptamer comprises an aptamer barcode sequence. In various embodiments, a method for internalization of a plurality of payloads into a cell can comprise confirming internalization of an internalization complex. In some embodiments, a confirming step can use at least one aptamer barcode sequence. In some embodiments, at least one aptamer is detectable. In some embodiments, an aptamer barcode sequence can be located on at least one aptamer.

In various embodiments, a method for internalization of a plurality of payloads into a cell can comprise sequencing the at least one aptamer barcode sequences.

In various embodiments, a method for internalization of a plurality of payloads into a cell can comprise confirming correct aptamer pairing. In some embodiments, confirming correct aptamer pairing comprises a fluorescence in situ hybridization method.

In various embodiments, a method for internalization of a plurality of payloads into a cell can comprise binding a capture sequence to at least one of the aptamers described herein. In some embodiments a capture sequence can be complementary to at least a portion of at least one aptamer. In some embodiments, the method can comprise a bead comprising an oligonucleotide. In some embodiments, an oligonucleotide can comprise a nucleic acid barcode molecule comprising a bead specific barcode. In some embodiments, an oligonucleotide can comprise a capture sequence. In some embodiments, an oligonucleotide further comprises a unique molecular identifier (UMI).

In various embodiments, a method can comprise partitioning the cell into a partition with a plurality of nucleic acid barcode molecules, wherein a nucleic acid barcode molecule of the plurality of nucleic acid barcode molecules comprises a partition-specific barcode sequence and a capture sequence that can be complementary to at least a portion of one of the aptamers. In various embodiments, the method can comprise hybridizing the capture sequence of the nucleic acid barcode molecule to the at least a portion of the one of the aptamers. In various embodiments, the method can comprise using the nucleic acid barcode molecule and the one of the aptamers to generate a barcoded product comprising the partition-specific barcode sequence or a complement thereof and the aptamer barcode sequence or a complement thereof.

In various embodiments, the method can comprise partitioning the cell and a bead into the partition, wherein the bead comprises the plurality of nucleic acid barcode molecules. In some embodiments, the nucleic acid barcode molecule of the plurality of nucleic acid barcode molecules further comprises a unique molecular identifier (UMI).

In various embodiments, the method can comprise determining the sequence of the barcoded product or a portion thereof. In some embodiments, the method can comprise confirming internalization of the internalization complex in the cell if the sequence of the barcoded product or portion thereof contains (i) the partition-specific barcode sequence or complement thereof and (ii) the aptamer barcode sequence or complement thereof.

In some embodiments, the method comprises generating one or more of the aptamers with a polynucleotide synthesis procedure.

In various embodiments, a method for internalization of a plurality of payloads into a cell can comprise generating one or more of the aptamers with a polynucleotide synthesis procedure.

In various embodiments, a method of preparing an internalization complex for internalization of a payload into a cell can comprise hybridizing a hybridization sequence of a first aptamer to a first hybridization sequence of a second aptamer and hybridizing a hybridization sequence of a third aptamer to a second hybridization sequence of the second aptamer, wherein the first aptamer comprises a 3′ end, a 5′ end, a cell surface binding domain, and the hybridization sequence of the first aptamer, wherein the second aptamer comprises a 3′ end, a 5′ end, the first hybridization sequence of the second aptamer, the second hybridization sequence of the second aptamer, and a payload binding domain, wherein the third aptamer comprises a 3′ end, a 5′ end, the hybridization sequence of the third aptamer, and a payload binding domain of the third aptamer. In various embodiments, the method can comprise binding the payload binding domain of the second aptamer to a payload, thereby preparing the internalization complex for internalization of the payload into the cell. In various embodiments, the method can comprise binding the payload binding domain of the third aptamer to an additional payload.

In various embodiments, a method of preparing an internalization complex for internalization of a payload into a cell can comprise hybridizing a first hybridization sequence of a first aptamer to a hybridization sequence of a second aptamer and hybridizing a second hybridization sequence of the first aptamer to a hybridization sequence of the third aptamer, wherein the first aptamer comprises a 3′ end, a 5′ end, a cell surface binding domain, the first hybridization sequence, and the second hybridization sequence, wherein the second aptamer comprises a 3′ end, a 5′ end, the hybridization sequence of the second aptamer, and a payload binding domain, wherein the third aptamer comprises a 3′ end, a 5′ end, the hybridization sequence of the third aptamer, and a payload binding domain of the third aptamer.

In various embodiments, the method can comprise binding the payload binding domain of the second aptamer to a payload, thereby preparing the internalization complex for internalization of the payload into the cell. In various embodiments, the method can comprise binding the payload binding domain of the third aptamer to an additional payload.

### IV. Kits

Aspects of the disclosure comprise kits for internalizing a payload into a cell. In various embodiments, kits can comprise a first aptamer and a second aptamer. In various embodiments, a first aptamer can comprise a 3′ end, 5′ end, cell surface binding domain, and a hybridization sequence. In various embodiments, a kit can comprise a second aptamer comprising, a 3′ end, a 5′ end, a payload binding domain, and a hybridization sequence. In various embodiments, the hybridization sequence of the first aptamer can be configured to hybridize to the hybridization sequence of the second aptamer. In various embodiments, a kit can comprise a payload.

In some embodiments, a first aptamer can comprise a detectable label. In some embodiments, a second aptamer can comprise a detectable label. In various embodiments, a detectable label comprises a fluorescent molecule. In various embodiments, a detectable label comprises a quenching agent configured to interact with a fluorescent molecule and decrease fluorescence. The aptamer, e.g., first and/or second aptamer, may comprise the detectable label as it may have, or may be engineered to include, a domain capable of comprising a detectable label as described elsewhere herein.

In various embodiments, aptamers can comprise aptamer barcode sequences.

In various embodiments, a payload binding domain can comprise a payload handle. In some embodiments, a payload handle can comprise a protein recognition sequence. In some embodiments, a payload handle can comprise a biotinylated structure. In some embodiments, a payload handle can comprise a poly-A tail capture sequence. In some embodiments, a payload handle can comprise a Cas molecule. In some embodiments, a payload handle can comprise a gRNA molecule. In some embodiments, a payload handle can comprise an RNP complex comprising a Cas molecule associated with a gRNA molecule. In some embodiments, a payload handle can comprise an mRNA molecule.

In some embodiments, a payload can comprise an oligonucleotide. In some embodiments, a payload can comprise a protein. In some embodiments, a payload can comprise an RNAi molecule.

In various embodiments, a kit can comprise a bead comprising an oligonucleotide. In some embodiments, an oligonucleotide can comprise a nucleic acid barcode molecule comprising a bead specific barcode. In some embodiments, an oligonucleotide can comprise a capture sequence. In some embodiments, a capture sequence can be complementary to at least a portion an aptamer. In some embodiments, an oligonucleotide can comprise a unique molecular identifier (UMI).

In various embodiments, a kit can comprise a first aptamer, a second aptamer, and a third aptamer. In various embodiments, a first aptamer can comprise a 3′ end, a 5′ end, a cell surface binding domain, and a hybridization sequence. In various embodiments, a second aptamer can comprise a 3′ end, a 5′ end, a first hybridization sequence, second hybridization sequence, and a payload binding domain. In various embodiments, a third aptamer can comprise a 3′ end, a 5′ end, a payload binding domain, and a hybridization sequence. In various embodiments, a hybridization sequence of a first aptamer can be configured to hybridize to a first hybridization sequence of a second aptamer, and a hybridization sequence of a third aptamer can be configured to hybridize to a second hybridization sequence of a second aptamer. In various embodiments, a kit can comprise a plurality of payloads.

In some embodiments, a first aptamer further comprises a detectable label. In some embodiments, a second aptamer further comprises a detectable label. In some embodiments, a third aptamer further comprises a detectable label. The aptamer, e.g., first and/or second and/or third aptamer, may comprise the detectable label via inclusion of a domain (or a variant of a domain) of an aptamer such as a spinach aptamer, broccoli aptamer, mango aptamer, corn aptamer, or variant thereof, that can bind a detectable label. In some embodiments, the detectable label comprises a fluorescent molecule. In some embodiments, a detectable label comprises a quenching agent configured to interact with a fluorescent molecule and decrease fluorescence.

In various embodiments, at least one of the aptamers comprises an aptamer barcode sequence.

In various embodiments, a payload binding domain further comprises a payload handle. In some embodiments, a payload handle comprises a protein recognition sequence. In some embodiments, a payload handle comprises a biotinylated structure. In some embodiments, a payload handle comprises a poly-A tail capture sequence. In some embodiments, a payload handle comprises a Cas molecule. In some embodiments, a payload handle comprises a gRNA molecule.

In some embodiments, a payload comprises an RNP complex. In some embodiments an RNP complex comprises a Cas molecule associated with a gRNA molecule. In some embodiments a payload comprises an mRNA molecule. In some embodiments a payload comprises an oligonucleotide. In some embodiments a payload comprises a protein. In some embodiments an RNAi molecule.

In various embodiments, kits can comprise a bead comprising an oligonucleotide. In some embodiments, an oligonucleotide can comprise a nucleic acid barcode molecule comprising a bead specific barcode. In some embodiments, an oligonucleotide can comprise a capture sequence. In some embodiments, a capture sequence can be complementary to at least a portion of at least one of the aptamers. In some embodiments, an oligonucleotide can further comprise a unique molecular identifier (UMI).

In various embodiments, a kit for internalizing a plurality of payloads into a cell can include a first aptamer, a second aptamer, and a third aptamer. In various embodiments, a first aptamer comprises a 3′ end, a 5′ end, a cell surface binding domain, a first hybridization sequence, and a second hybridization sequence. In various embodiments, a second aptamer comprises a 3′ end, a 5′ end, a hybridization sequence, and a payload binding domain. In various embodiments, a third aptamer comprises a 3′ end, a 5′ end, a hybridization sequence, and a payload binding domain. In various embodiments, a first hybridization sequence can be configured to hybridize to a hybridization sequence of the second aptamer. In various embodiments, a second hybridization sequence can be configured to hybridize to a hybridization sequence of a third aptamer. In various embodiments, a kit can comprise a plurality of payloads.

In various embodiments, a first aptamer can comprise a detectable label. In various embodiments, a second aptamer can comprise a detectable label. In various embodiments, a third aptamer can comprise a detectable label. The aptamer, e.g., first and/or second and/or third aptamer, may comprise the detectable label via inclusion of a domain (or a variant of a domain) of an aptamer such as a spinach aptamer, broccoli aptamer, mango aptamer, corn aptamer, or variant thereof, that can bind a detectable label. In some embodiments, a detectable label comprises a fluorescent molecule. In some embodiments, a detectable label comprises a quenching agent configured to interact with a fluorescent molecule and decrease fluorescence.

In various embodiments, an aptamer can comprise an aptamer barcode sequence.

In various embodiments, a payload binding domain further comprises a payload handle. In some embodiments, a payload handle comprises a protein recognition sequence. In some embodiments, a payload handle comprises a biotinylated structure. In some embodiments, a payload handle comprises a poly-A tail capture sequence.

In various embodiments, a payload comprises a Cas molecule. In various embodiments, a payload comprises a gRNA molecule. In various embodiments, a payload comprises an RNP complex. In various embodiments, an RNP complex comprises a Cas molecule associated with a gRNA molecule. In various embodiments, a payload comprises an mRNA molecule. In various embodiments, a payload comprises an oligonucleotide. In various embodiments, a payload comprises a protein. In various embodiments, a payload comprises an RNAi molecule.

In various embodiments, a kit can comprise a bead comprising an oligonucleotide. In various embodiments, an oligonucleotide can comprise a nucleic acid barcode molecule comprising a bead specific barcode. In various embodiments, an oligonucleotide can comprise a capture sequence. In various embodiments, a capture sequence can be complementary to at least a portion of an aptamer. In some embodiments, an oligonucleotide further comprises a unique molecular identifier (UMI).

### V. Systems and Methods for Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. A partition can be a volume wherein diffusion of contents beyond the volume is inhibited. For example, the partitions can include a porous matrix that is capable of entraining and/or retaining materials within its matrix. In particular embodiments, biological particles include cells or macromolecular constituents of cells that have been contacted with an aptamer-payload complex or internalization complex disclosed herein, e.g., depicted in FIG. 1, 13, 14, 15. The partition can be a droplet in an emulsion or a well. A partition can comprise one or more other partitions.

A partition can include one or more particles. A partition can include one or more types of particles. For example, a partition of the present disclosure can comprise one or more biological particles and/or macromolecular constituents thereof. A partition can comprise one or more beads. A partition can comprise one or more gel beads. A partition can comprise one or more cell beads. A partition can include a single gel bead, a single cell bead, or both a single cell bead and single gel bead. A partition can include one or more reagents. Alternatively, a partition can be unoccupied. For example, a partition may not comprise a bead.

Unique identifiers, such as barcodes, can be injected into the droplets prior to, subsequent to, or concurrently with droplet generation, such as via a bead, as described elsewhere herein.

The methods and systems of the present disclosure can comprise methods and systems for generating one or more partitions such as droplets. The droplets can comprise a plurality of droplets in an emulsion. In various examples, the droplets can comprise droplets in a colloid. In various cases, the emulsion can comprise a microemulsion or a nanoemulsion. In various examples, the droplets can be generated with aid of a microfluidic device and/or by subjecting a mixture of immiscible phases to agitation (e.g., in a container). In various cases, a combination of the mentioned methods can be used for droplet and/or emulsion formation.

The partitions described herein may comprise small volumes, for example, less than about 10 microliters (μL), 5 μL, 1 μL, 10 nanoliters (nL), 5 nL, 1 nL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet-based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with beads, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

Droplets can be formed by creating an emulsion by mixing and/or agitating immiscible phases. Mixing or agitation may comprise various agitation techniques, such as vortexing, pipetting, tube flicking, or other agitation techniques. In various cases, mixing or agitation may be performed without using a microfluidic device. In various examples, the droplets may be formed by exposing a mixture to ultrasound or sonication. Systems and methods for droplet and/or emulsion generation by agitation are described in International Application No. PCT/US20/17785, which is entirely incorporated by reference herein for all purposes.

### Microfluidic Systems:

Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particles in partitions, methods of increasing the throughput of droplet generation, and various geometries, architectures, and configurations of microfluidic devices and channels are described in U.S. Patent Publication Nos. 2019/0367997 and 2019/0064173, each of which is entirely incorporated herein by reference for all purposes.

In various examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as biological particles, in various cases, individual biological particles such as single cells. In various examples, reagents may be encapsulated and/or partitioned (e.g., co-partitioned with biological particles) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In various cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In various examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In various cases, partitions among a plurality of partitions may contain at most one biological particle (e.g., bead, DNA, cell or cellular material). In various embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

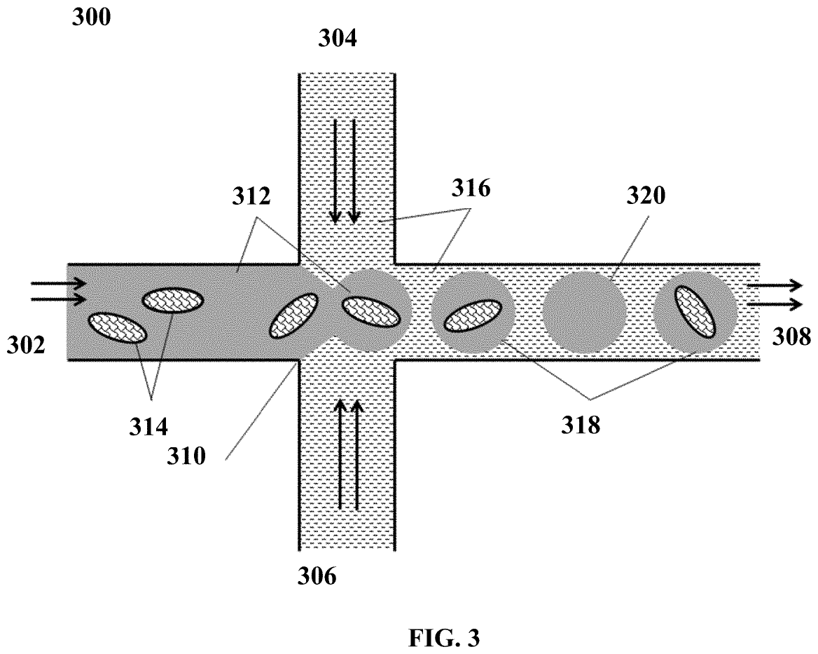

FIG. 3 shows an example of a microfluidic channel structure 306 for partitioning individual biological particles (e.g., cells or macromolecular constituents of cells that have been contacted with an aptamer-payload complex or internalization complex disclosed herein, e.g., depicted in FIG. 1, 13, 14, 15) according to various embodiments. The channel structure 306 can include channel segments 302, 304, 306 and 308 communicating at a channel junction 310. In operation, a first aqueous fluid 312 that includes suspended biological particles (or cells) 314 may be transported along channel segment 302 into junction 310, while a second fluid 316 that is immiscible with the aqueous fluid 312 is delivered to the junction 310 from each of channel segments 304 and 306 to create discrete droplets 318, 320 of the first aqueous fluid 312 flowing into channel segment 308, and flowing away from junction 310. The channel segment 308 may be fluidically coupled to an outlet reservoir where the discrete droplets can be stored and/or harvested. A discrete droplet generated may include an individual biological particle 314 (such as droplets 318). A discrete droplet generated may include more than one individual biological particle 314 (not shown in FIG. 3). A discrete droplet may contain no biological particle 314 (such as droplet 320). Each discrete partition may maintain separation of its own contents (e.g., individual biological particle 314) from the contents of other partitions.

The second fluid 316 can comprise an oil, such as a fluorinated oil, that includes a fluorosurfactant for stabilizing the resulting droplets, for example, inhibiting subsequent coalescence of the resulting droplets 318, 320. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 318, containing one or more biological particles 314, and (2) unoccupied droplets 320, not containing any biological particles 314. Occupied droplets 318 may comprise singly occupied droplets (having one biological particle) and multiply occupied droplets (having more than one biological particle). As described elsewhere herein, in various cases, the majority of occupied partitions can include no more than one biological particle per occupied partition and various of the generated partitions can be unoccupied (of any biological particle). In various cases, though, various of the occupied partitions may include more than one biological particle. In various cases, the partitioning process may be controlled such that fewer than about 25% of the occupied partitions contain more than one biological particle, and in many cases, fewer than about 20% of the occupied partitions have more than one biological particle, while in various cases, fewer than about 10% or even fewer than about 5% of the occupied partitions include more than one biological particle per partition.

In various cases, it may be desirable to minimize the creation of excessive numbers of empty partitions, such as to reduce costs and/or increase efficiency. While this minimization may be achieved by providing a sufficient number of biological particles (e.g., biological particles 314) at the partitioning junction 310, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In various cases, flows can be controlled so as to present a non-Poissonian distribution of single-occupied partitions while providing lower levels of unoccupied partitions (e.g., no more than about 50%, about 25%, or about 10% unoccupied). The above noted ranges of unoccupied partitions can be achieved while still providing any of the single occupancy rates described above.

As will be appreciated, the above-described occupancy rates are also applicable to partitions that include both biological particles and additional reagents, such as beads (e.g., gel beads) carrying nucleic acid barcode molecules (e.g., oligonucleotides).

In various examples, a partition of the plurality of partitions may comprise a single biological particle (e.g., a single cell or a single nucleus of a cell). In various examples, a partition of the plurality of partitions may comprise multiple biological particles. Such partitions may be referred to as multiply occupied partitions, and may comprise, for example, two, three, four or more cells and/or beads (e.g., beads) comprising nucleic acid barcode molecules within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in various cases greater than about 80%, 90%, 95%, or higher.

Microfluidic systems for partitioning are further described in U.S. Patent Application Pub. No. US 2015/0376609, which is hereby incorporated by reference in its entirety.

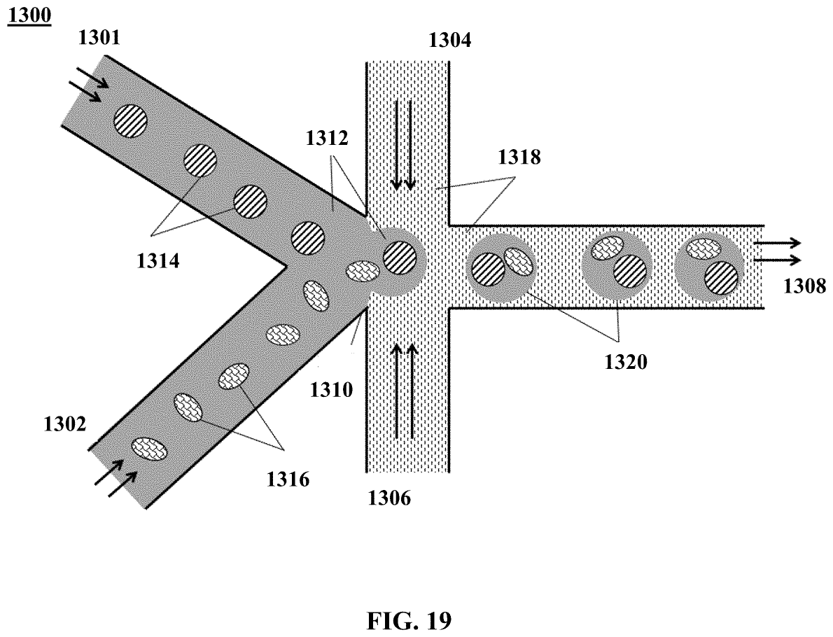

FIG. 19 shows an example of a microfluidic channel structure 1300 for delivering barcode carrying beads to droplets. The channel structure 1300 can include channel segments 1301, 1302, 1304, 1306 and 1308 communicating at a channel junction 1310. In operation, the channel segment 1301 may transport an aqueous fluid 1312 that includes a plurality of beads 1314 (e.g., with nucleic acid molecules, e.g., nucleic acid barcode molecules or barcoded oligonucleotides, molecular tags) along the channel segment 1301 into junction 1310. The plurality of beads 1314 may be sourced from a suspension of beads. For example, the channel segment 1401 may be connected to a reservoir comprising an aqueous suspension of beads 1314. The channel segment 1302 may transport the aqueous fluid 1312 that includes a plurality of biological particles 1316 (e.g., cells or macromolecular constituents of cells that have been contacted with an aptamer-payload complex or internalization complex disclosed herein, e.g., depicted in FIG. 1, 13, 14, 15) along the channel segment 1302 into junction 1310. The plurality of biological particles 1316 may be sourced from a suspension of biological particles. For example, the channel segment 1302 may be connected to a reservoir comprising an aqueous suspension of biological particles 1316. In some instances, the aqueous fluid 1312 in either the first channel segment 1301 or the second channel segment 1302, or in both segments, can include one or more reagents, as further described below. A second fluid 1318 that is immiscible with the aqueous fluid 1312 (e.g., oil) can be delivered to the junction 1310 from each of channel segments 1304 and 1406. Upon meeting of the aqueous fluid 1312 from each of channel segments 1301 and 1402 and the second fluid 1318 from each of channel segments 1304 and 1306 at the channel junction 1310, the aqueous fluid 1312 can be partitioned as discrete droplets 1420 in the second fluid 1318 and flow away from the junction 1310 along channel segment 1308. The channel segment 1308 may deliver the discrete droplets to an outlet reservoir fluidly coupled to the channel segment 1308, where they may be harvested. As an alternative, the channel segments 1301 and 1302 may meet at another junction upstream of the junction 1310. At such junction, beads and biological particles may form a mixture that is directed along another channel to the junction 1310 to yield droplets 1420. The mixture may provide the beads and biological particles in an alternating fashion, such that, for example, a droplet comprises a single bead and a single biological particle.

### Controlled Partitioning:

In various aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

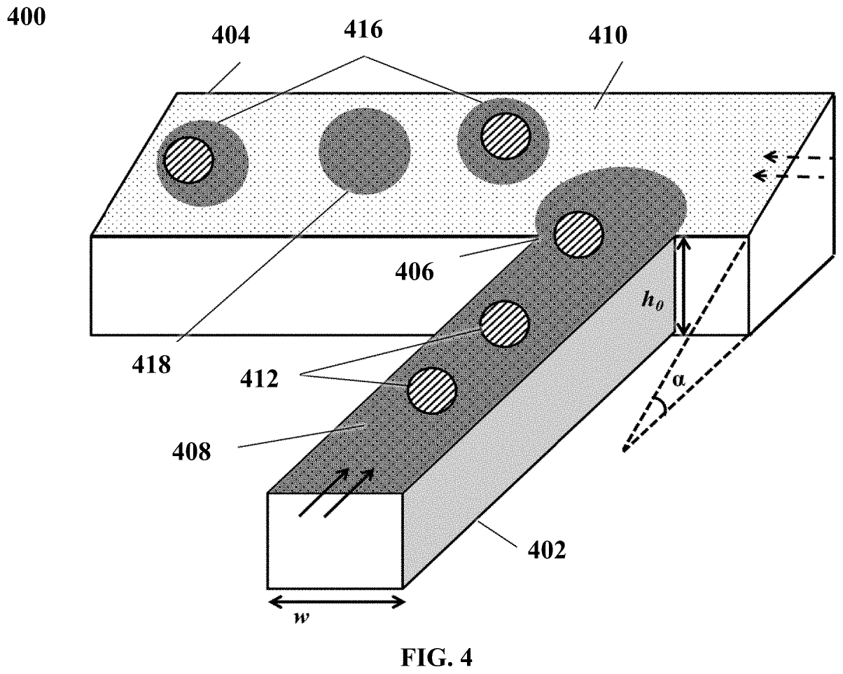

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., ho, a, etc.) of the channel structure 400. A plurality of droplets can be collected in the reservoir 404 by continuously injecting the aqueous fluid 408 from the channel segment 402 through the junction 406.

In various instances, the aqueous fluid 408 can have a substantially uniform concentration or frequency of beads 412. The beads 412 can be introduced into the channel segment 402 from a separate channel (not shown in FIG. 4). The frequency of beads 412 in the channel segment 402 may be controlled by controlling the frequency in which the beads 412 are introduced into the channel segment 202 and/or the relative flow rates of the fluids in the channel segment 402 and the separate channel. In various instances, the beads can be introduced into the channel segment 402 from a plurality of different channels, and the frequency controlled accordingly.

In various instances, the aqueous fluid 408 in the channel segment 402 can comprise biological particles (e.g., cells or macromolecular constituents of cells that have been contacted with an aptamer-payload complex or internalization complex disclosed herein, e.g., depicted in FIG. 1, 13, 14, 15). In various instances, the aqueous fluid 408 can have a substantially uniform concentration or frequency of biological particles. As with the beads, the biological particles can be introduced into the channel segment 402 from a separate channel. The frequency or concentration of the biological particles in the aqueous fluid 408 in the channel segment 402 may be controlled by controlling the frequency in which the biological particles are introduced into the channel segment 402 and/or the relative flow rates of the fluids in the channel segment 402 and the separate channel. In various instances, the biological particles can be introduced into the channel segment 402 from a plurality of different channels, and the frequency controlled accordingly. In various instances, a first separate channel can introduce beads and a second separate channel can introduce biological particles into the channel segment 402. The first separate channel introducing the beads may be upstream or downstream of the second separate channel introducing the biological particles.

The second fluid 410 can comprise an oil, such as a fluorinated oil, that includes a fluorosurfactant for stabilizing the resulting droplets, for example, inhibiting subsequent coalescence of the resulting droplets.

In various instances, the second fluid 410 may not be subjected to and/or directed to any flow in or out of the reservoir 404. For example, the second fluid 410 may be substantially stationary in the reservoir 404. In various instances, the second fluid 410 may be subjected to flow within the reservoir 404, but not in or out of the reservoir 404, such as via application of pressure to the reservoir 404 and/or as affected by the incoming flow of the aqueous fluid 408 at the junction 406. Alternatively, the second fluid 410 may be subjected and/or directed to flow in or out of the reservoir 404. For example, the reservoir 404 can be a channel directing the second fluid 410 from upstream to downstream, transporting the generated droplets.

Systems and methods for controlled partitioning are described further in PCT/US2018/047551, the disclosure of which is hereby incorporated by reference in its entirety.

### Beads:

Nucleic acid barcode molecules may be delivered to a partition (e.g., a droplet or well) via a solid support or carrier (e.g., a bead). In various cases, nucleic acid barcode molecules are initially associated with the solid support and then released from the solid support upon application of a stimulus, which allows the nucleic acid barcode molecules to dissociate or to be released from the solid support. In specific examples, nucleic acid barcode molecules are initially associated with the solid support (e.g., bead) and then released from the solid support upon application of a biological stimulus, a chemical stimulus, a thermal stimulus, an electrical stimulus, a magnetic stimulus, and/or a photo stimulus.

The solid support may be a bead. A solid support, e.g., a bead, may be porous, non-porous, hollow, solid, semi-solid, and/or a combination thereof. Beads may be solid, semi-solid, semi-fluidic, fluidic, and/or a combination thereof. In various instances, a solid support, e.g., a bead, may be at least partially dissolvable, disruptable, and/or degradable. In various cases, a solid support, e.g., a bead, may not be degradable. In various cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In various cases, the solid support, e.g., the bead, may be a silica bead. In various cases, the solid support, e.g., a bead, can be rigid. In other cases, the solid support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., via a nucleic acid barcode molecule), to a partition via any suitable mechanism. Nucleic acid barcode molecules can be delivered to a partition via a bead. Beads are described in further detail below.

In various cases, nucleic acid barcode molecules can be initially associated with the bead and then released from the bead. Release of the nucleic acid barcode molecules can be passive (e.g., by diffusion out of the bead). In addition or alternatively, release from the bead can be upon application of a stimulus which allows the nucleic acid barcode molecules to dissociate or to be released from the bead. Such stimulus may disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

Methods and systems for partitioning barcode carrying beads into droplets are provided herein, and in in US. Patent Publication Nos. 2019/0367997 and 2019/0064173, and International Application No. PCT/US20/17785, the disclosure of which are herein incorporated by reference in their entireties for all purposes.

A bead may be porous, non-porous, solid, semi-solid, semi-fluidic, fluidic, and/or a combination thereof. In various instances, a bead may be dissolvable, disruptable, and/or degradable. Degradable beads, as well as methods for degrading beads, are described in PCT/US2014/044398, the disclosure of which is hereby incorporated by reference in its entirety. In various cases, any combination of stimuli, e.g., stimuli described in PCT/US2014/044398 and US Patent Application Pub. No. 2015/0376609, the disclosures of which are hereby incorporated by reference in their entireties, may trigger degradation of a bead. For example, a change in pH may enable a chemical agent (e.g., DTT) to become an effective reducing agent.

In various cases, a bead may not be degradable. In various cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In various cases, the bead may be a silica bead. In various cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In various cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In various cases, a bead may have a diameter of less than about 10 nm, 100 nm, 500 nm, 1 μm, 10 μm, 20 μm, 30 μm, 40 μm, 50 μm, 60 μm, 70 μm, 80 μm, 90 μm, 100 μm, 250 μm, 500 μm, 1 mm, or less. In various cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in various cases less than 15%, less than 10%, less than 5%, or less.

A bead may comprise natural and/or synthetic materials. For example, a bead can comprise a natural polymer, a synthetic polymer or both natural and synthetic polymers. See, e.g., PCT/US2014/044398, the disclosure of which is hereby incorporated by reference in its entirety. Beads may also be formed from materials other than polymers, including lipids, micelles, ceramics, glass-ceramics, material composites, metals, other inorganic materials, and others.

In various cases, the bead may comprise covalent or ionic bonds between polymeric precursors (e.g., monomers, oligomers, linear polymers), nucleic acid barcode molecules (e.g., oligonucleotides), primers, and other entities. In various cases, the covalent bonds can be carbon-carbon bonds, thioether bonds, or carbon-heteroatom bonds.

In various cases, a plurality of nucleic acid barcode molecules may be attached to a bead. The nucleic acid barcode molecules may be attached directly or indirectly to the bead. In various cases, the nucleic acid barcode molecules may be covalently linked to the bead. In various cases, the nucleic acid barcode molecules are covalently linked to the bead via a linker. In various cases, the linker is a degradable linker. In various cases, the linker comprises a labile bond configured to release said nucleic acid barcode molecule of said plurality of nucleic acid barcode molecules. In various cases, the labile bond comprises a disulfide linkage.

Activation of disulfide linkages within a bead can be controlled such that only a small number of disulfide linkages are activated. Methods of controlling activation of disulfide linkages within a bead are described in PCT/US2014/044398, the disclosure of which is hereby incorporated by reference in its entirety.

In various cases, a bead may comprise an acrydite moiety, which in certain aspects may be used to attach one or more nucleic acid barcode molecules (e.g., barcode sequence, nucleic acid barcode molecule, barcoded oligonucleotide, primer, or other oligonucleotide) to the bead. Acrydite moieties, as well as their uses in attaching nucleic acid molecules to beads, are described in PCT/US2014/044398, the disclosure of which is hereby incorporated by reference in its entirety.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule, e.g., a nucleic acid barcode molecule described herein.

In various cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. Exemplary precursors comprising functional groups are described in PCT/US2014/044398, the disclosure of which is hereby incorporated by reference in its entirety.

Other non-limiting examples of labile bonds that may be coupled to a precursor or bead are described in PCT/US2014/044398, the disclosure of which is hereby incorporated by reference in its entirety. A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. See, e.g., PCT/US2014/044398, the disclosure of which is hereby incorporated by reference in its entirety. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Alternatively, or in addition, species may be partitioned in a partition (e.g., droplet) during or subsequent to partition formation. Such species may include, without limitation, the abovementioned species that may also be encapsulated in a bead.

In various cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In various cases, the pre-defined concentration of the primer can be limited by the process of producing oligonucleotide bearing beads.

### Nucleic Acid Barcode Molecules:

A nucleic acid barcode molecule may contain one or more barcode sequences. A plurality of nucleic acid barcode molecules may be coupled to a bead. The one or more barcode sequences may include sequences that are the same for all nucleic acid molecules coupled to a given bead and/or sequences that are different across all nucleic acid molecules coupled to the given bead. The nucleic acid molecule may be incorporated into the bead.

Nucleic acid barcode molecules can comprise one or more functional sequences for coupling to an analyte or analyte tag such as a reporter oligonucleotide. Such functional sequences can include, e.g., a template switch oligonucleotide (TSO) sequence, a primer sequence (e.g., a poly T sequence, or a nucleic acid primer sequence complementary to a target nucleic acid sequence and/or for amplifying a target nucleic acid sequence, a random primer, and a primer sequence for messenger RNA).

In various cases, the nucleic acid barcode molecule can further comprise a unique molecular identifier (UMI). In various cases, the nucleic acid barcode molecule can comprise one or more functional sequences, for example, for attachment to a sequencing flow cell, such as, for example, a P5 sequence (or a portion thereof) for Illumina® sequencing. In various cases, the nucleic acid barcode molecule or derivative thereof (e.g., oligonucleotide or polynucleotide generated from the nucleic acid molecule) can comprise another functional sequence, such as, for example, a P7 sequence (or a portion thereof) for attachment to a sequencing flow cell for Illumina sequencing. In various cases, the nucleic acid molecule can comprise an R1 primer sequence for Illumina sequencing. In various cases, the nucleic acid molecule can comprise an R2 primer sequence for Illumina sequencing. In various cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In various cases, a downstream procedure may extend the partial sequence, or derivative thereof, to achieve a full sequence of the partial sequence, or derivative thereof.

Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, the disclosures of which are incorporated by reference herein in their entireties.

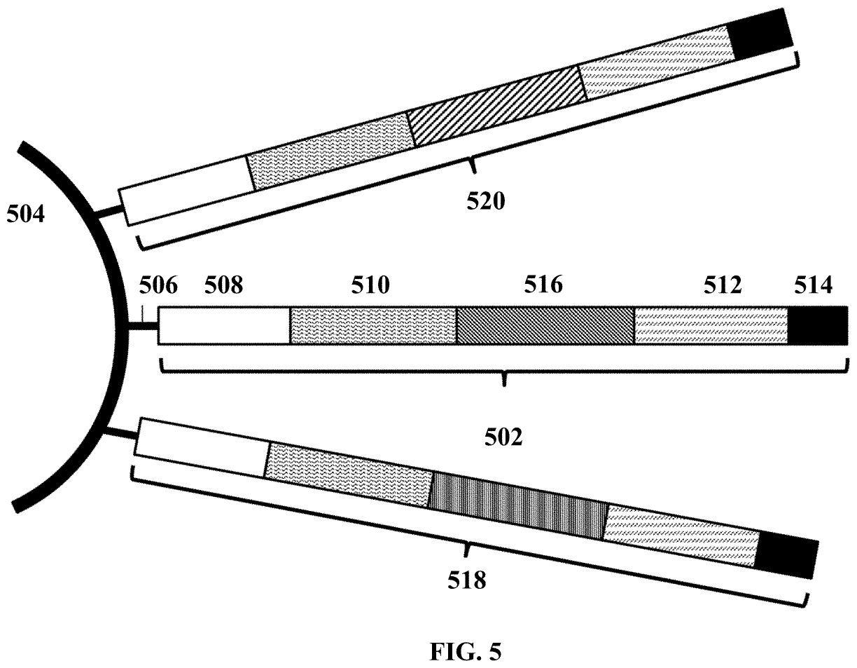

FIG. 5 illustrates an example of a barcode carrying bead. A nucleic acid barcode molecule 502 can be coupled to a bead 504 by a releasable linkage 506, such as, for example, a disulfide linker. The same bead 504 may be coupled (e.g., via releasable linkage) to one or more other nucleic acid barcode molecules 518, 520. The nucleic acid barcode molecule 502 may be or comprise a barcode. As noted elsewhere herein, the structure of the barcode may comprise a number of sequence elements. The nucleic acid barcode molecule 502 may comprise a functional sequence 508 that may be used in subsequent processing. For example, the functional sequence 508 may include one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof. The nucleic acid barcode molecule 502 may comprise a barcode sequence 510 for use in barcoding the sample (e.g., DNA, RNA, protein, etc.). In various cases, the barcode sequence 510 can be bead-specific such that the barcode sequence 510 is common to all nucleic acid barcode molecules (e.g., including nucleic acid barcode molecule 502) coupled to the same bead 504. Alternatively or in addition, the barcode sequence 510 can be partition-specific such that the barcode sequence 510 is common to all nucleic acid barcode molecules coupled to one or more beads that are partitioned into the same partition. The nucleic acid barcode molecule 502 may comprise sequence 512 complementary to an analyte of interest, e.g., a priming sequence. Sequence 512 can be a poly-T sequence complementary to a poly-A tail of an mRNA analyte, a targeted priming sequence, and/or a random priming sequence. The nucleic acid barcode molecule 502 may comprise an anchoring sequence 514 to ensure that the specific priming sequence 512 hybridizes at the sequence end (e.g., of the mRNA). For example, the anchoring sequence 514 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid barcode molecule 502 may comprise a unique molecular identifying (UMI) sequence 516. In various cases, the unique molecular identifying sequence 516 may comprise from about 5 to about 8 nucleotides. Alternatively, the unique molecular identifying sequence 516 may compress less than about 5 or more than about 8 nucleotides. The unique molecular identifying sequence 516 may be a unique sequence that varies across individual nucleic acid barcode molecules (e.g., 502, 518, 520, etc.) coupled to a single bead (e.g., bead 504). In various cases, the unique molecular identifying sequence 516 may be a random sequence (e.g., such as a random N-mer sequence). For example, the UMI 516 may provide a unique identifier of the starting analyte (e.g., mRNA) molecule that was captured, in order to allow quantitation of the number of original expressed RNA molecules. As will be appreciated, although FIG. 5 shows three nucleic acid barcode molecules 502, 518, 520 coupled to the surface of the bead 504, an individual bead may be coupled to any number of individual nucleic acid barcode molecules, for example, from one to tens to hundreds of thousands, millions, or even a billion of individual nucleic acid barcode molecules. The respective barcodes for the individual nucleic acid barcode molecules can comprise both common sequence segments or relatively common sequence segments (e.g., 508, 510, 512, etc.) and variable or unique sequence segments (e.g., 516) between different individual nucleic acid barcode molecules coupled to the same bead.

In operation, a biological particle (e.g., cell, DNA, RNA, etc.) can be co-partitioned along with a barcode bearing bead 504. The nucleic acid barcode molecules 502, 518, 520 can be released from the bead 504 in the partition. By way of example, in the context of analyzing sample RNA, the poly-T segment (e.g., 512) of one of the released nucleic acid barcode molecules (e.g., 502) can hybridize to the poly-A tail of a mRNA molecule. Reverse transcription may result in a cDNA transcript of the mRNA, but which transcript includes each of the sequence segments 508, 510, 516 of the nucleic acid barcode molecule 502. Because the nucleic acid barcode molecule 502 comprises an anchoring sequence 514, it will more likely hybridize to and prime reverse transcription at the sequence end of the poly-A tail of the mRNA. Within any given partition, all of the cDNA transcripts of the individual mRNA molecules may include a common barcode sequence segment 510. However, the transcripts made from the different mRNA molecules within a given partition may vary at the unique molecular identifying sequence 516 segment (e.g., UMI segment). Beneficially, even following any subsequent amplification of the contents of a given partition, the number of different UMIs 516 can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in various cases, the nucleic acid barcode molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where analytes from the sample are captured by the nucleic acid barcode molecules in a partition (e.g., by hybridization), captured analytes from various partitions may be collected, pooled, and subjected to further processing (e.g., reverse transcription, adapter attachment, amplification, clean up, sequencing). For example, in cases wherein the nucleic acid molecules from the sample remain attached to the bead, the beads from various partitions may be collected, pooled, and subjected to further processing (e.g., reverse transcription, adapter attachment, amplification, clean up, sequencing). In other instances, one or more of the processing methods, e.g., reverse transcription, may occur in the partition. For example, conditions sufficient for barcoding, adapter attachment, reverse transcription, or other nucleic acid processing operations may be provided in the partition and performed prior to clean up and sequencing.

In various instances, a bead may comprise a capture sequence or binding sequence configured to bind to a corresponding capture sequence or binding sequence. In various instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, etc. A bead may comprise any number of different capture sequences. In various instances, a bead may comprise at least 2, 3, 4, 5, 6, 7, 8, 9, 10 or more different capture sequences or binding sequences configured to bind to different respective capture sequences or binding sequences, respectively. Alternatively or in addition, a bead may comprise at most about 10, 9, 8, 7, 6, 5, 4, 3, or 2 different capture sequences or binding sequences configured to bind to different respective capture sequences or binding sequences. In various instances, the different capture sequences or binding sequences may be configured to facilitate analysis of a same type of analyte. In various instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, an biological particle (e.g., cell, cell bead, etc.) for performing different assays in various formats (e.g., barcoded antibodies comprising the corresponding capture sequence, barcoded WIC dextramers comprising the corresponding capture sequence, barcoded guide RNA molecules comprising the corresponding capture sequence, etc.), such that the corresponding capture sequence may later interact with the capture sequence associated with the bead. In various instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules.

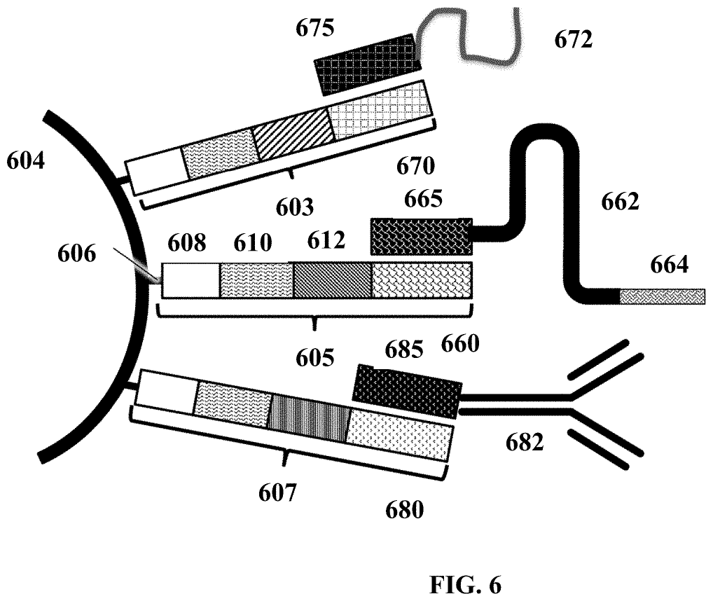

FIG. 6 illustrates another example of a barcode carrying bead. A nucleic acid barcode molecule 605, such as an oligonucleotide, can be coupled to a bead 604 by a releasable linkage 606, such as, for example, a disulfide linker. The nucleic acid barcode molecule 605 may comprise a first capture sequence 660. The same bead 604 may be coupled (e.g., via releasable linkage) to one or more other nucleic acid molecules 603, 607 comprising other capture sequences. The nucleic acid barcode molecule 605 may be or comprise a barcode. As noted elsewhere herein, the structure of the barcode may comprise a number of sequence elements, such as a functional sequence 608 (e.g., flow cell attachment sequence, sequencing primer sequence, etc.), a barcode sequence 610 (e.g., bead-specific sequence common to bead, partition-specific sequence common to partition, etc.), and a unique molecular identifier 612 (e.g., unique sequence within different molecules attached to the bead), or partial sequences thereof. The capture sequence 660 may be configured to attach to a corresponding capture sequence 665. In various instances, the corresponding capture sequence 665 may be coupled to another molecule that may be an analyte or an intermediary carrier. For example, as illustrated in FIG. 6, the corresponding capture sequence 665 is coupled to a guide RNA molecule 662 comprising a target sequence 664, wherein the target sequence 664 is configured to attach to the analyte. Another oligonucleotide molecule 607 attached to the bead 604 comprises a second capture sequence 680 which is configured to attach to a third corresponding capture sequence 685. As illustrated in FIG. 6, the second corresponding capture sequence 685 is coupled to an antibody 682. In various cases, the antibody 682 may have binding specificity to an analyte (e.g., surface protein). Alternatively, the antibody 682 may not have binding specificity. Another oligonucleotide molecule 603 attached to the bead 604 comprises a third capture sequence 670 which is configured to attach to a second corresponding capture sequence 675. As illustrated in FIG. 6, the third corresponding capture sequence 675 is coupled to a molecule 672. The molecule 672 may or may not be configured to target an analyte. The other oligonucleotide molecules 603, 607 may comprise the other sequences (e.g., functional sequence, barcode sequence, UMI, etc.) described with respect to oligonucleotide molecule 605. While a single oligonucleotide molecule comprising each capture sequence is illustrated in FIG. 6, it will be appreciated that, for each capture sequence, the bead may comprise a set of one or more oligonucleotide molecules each comprising the capture sequence. For example, the bead may comprise any number of sets of one or more different capture sequences. Alternatively, or in addition, the bead 604 may comprise other capture sequences. Alternatively, or in addition, the bead 604 may comprise fewer types of capture sequences (e.g., two capture sequences). Alternatively, or in addition, the bead 604 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. See, e.g., PCT/US2014/044398, the disclosure of which is hereby incorporated by reference in its entirety.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. See, e.g., PCT/US2014/044398, the disclosure of which is hereby incorporated by reference in its entirety.

As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In various cases, a species (e.g., oligonucleotide molecules comprising barcodes) that are attached to a solid support (e.g., a bead) may comprise a U-excising element that allows the species to release from the bead. In various cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In various cases, the length of a barcode sequence may be about 6, 7, 8, 9, 10, 11, 12, 13, 14, 15, 16, 17, 18, 19, 20 nucleotides or longer. In various cases, the length of a barcode sequence may be at least about 6, 7, 8, 9, 10, 11, 12, 13, 14, 15, 16, 17, 18, 19, 20 nucleotides or longer. In various cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In various cases, separated barcode subsequences can be from about 4 to about 16 nucleotides in length. In various cases, the barcode subsequence may be about 4, 5, 6, 7, 8, 9, 10, 11, 12, 13, 14, 15, 16 nucleotides or longer. In various cases, the barcode subsequence may be at least about 4, 5, 6, 7, 8, 9, 10, 11, 12, 13, 14, 15, 16 nucleotides or longer. In various cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, beads, such as beads, are provided that each include large numbers of the above described nucleic acid barcode molecules releasably attached to the beads, where all of the nucleic acid barcode molecules attached to a particular bead will include a common nucleic acid barcode sequence, but where a large number of diverse barcode sequences are represented across the population of beads used. In various embodiments, hydrogel beads, e.g., comprising polyacrylamide polymer matrices, are used as a solid support and delivery vehicle for the nucleic acid barcode molecules into the partitions, as they are capable of carrying large numbers of nucleic acid barcode molecules, and may be configured to release those nucleic acid molecules upon exposure to a particular stimulus, as described elsewhere herein. In various cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In various cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in various cases at least about 1 billion nucleic acid molecules, or more. In various embodiments, the number of nucleic acid molecules including the barcode sequence on an individual bead is between about 1,000 to about 10,000 nucleic acid molecules, about 5,000 to about 50,000 nucleic acid molecules, about 10,000 to about 100,000 nucleic acid molecules, about 50,000 to about 1,000,000 nucleic acid molecules, about 100,000 to about 10,000,000 nucleic acid molecules, about 1,000,000 to about 1 billion nucleic acid molecules.

Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acids, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in various cases at least about 1 billion nucleic acid barcode molecules.

In various cases, the resulting population of partitions provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences. Additionally, each partition of the population can include between about 1,000 to about 10,000 nucleic acid barcode molecules, about 5,000 to about 50,000 nucleic acid barcode molecules, about 10,000 to about 100,000 nucleic acid barcode molecules, about 50,000 to about 1,000,000 nucleic acid barcode molecules, about 100,000 to about 10,000,000 nucleic acid barcode molecules, about 1,000,000 to about 1 billion nucleic acid barcode molecules.

In various cases, it may be desirable to incorporate multiple different barcodes within a given partition, either attached to a single or multiple beads within the partition. For example, in various cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In various cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

### Reagents:

In accordance with certain aspects, biological particles (e.g., cells or macromolecular constituents of cells that have been contacted with an aptamer-payload complex or internalization complex disclosed herein, e.g., depicted in FIG. 1, 13, 14, 15) may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 410), such as through an additional channel or channels upstream of the channel junction. In accordance with other aspects, additionally or alternatively, biological particles may be partitioned along with other reagents, as will be described further below.

The methods and systems of the present disclosure may comprise microfluidic devices and methods of use thereof, which may be used for co-partitioning biological particles with reagents. Such systems and methods are described in U.S. Patent Publication No. US/20190367997, the disclosure of which is herein incorporated by reference in its entirety for all purposes.

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments of the microfluidic devices described elsewhere herein may be coupled to any of a variety of different fluid sources or receiving components, including reservoirs, tubing, manifolds, or fluidic components of other systems. As will be appreciated, the microfluidic channel structures may have various geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in various cases, surfactant-based lysis solutions may be used to lyse cells, although these may be less desirable for emulsion-based systems where the surfactants can interfere with stable emulsions. In various cases, lysis solutions may include non-ionic surfactants such as, for example, TritonX-100 and Tween 20. In various cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion-based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., a cell or a nucleus in a polymer matrix), the biological particles may be exposed to an appropriate stimulus to release the biological particles or their contents from a co-partitioned bead. For example, in various cases, a chemical stimulus may be co-partitioned along with an encapsulated biological particle to allow for the degradation of the bead and release of the cell or its contents into the larger partition. In various cases, this stimulus may be the same as the stimulus described elsewhere herein for release of nucleic acid molecules (e.g., oligonucleotides) from their respective bead. In alternative examples, this may be a different and non-overlapping stimulus, in order to allow an encapsulated biological particle to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. No. 20190100632, the disclosures of which are hereby incorporated by reference in their entireties.

Additional reagents may also be co-partitioned with the biological particle, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In various cases, template switching can be used to increase the length of a cDNA. In various cases, template switching can be used to append a predefined nucleic acid sequence to the cDNA. Template switching is further described in PCT/US2017/068320, the disclosure of which is hereby incorporated by reference in its entirety. Template switching oligonucleotides may comprise a hybridization region and a template region. Template switching oligonucleotides are further described in PCT/US2017/068320, the disclosure of which is hereby incorporated by reference in its entirety.

Any of the reagents described in this disclosure may be encapsulated in, or otherwise coupled to, a droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, and oligonucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles. In various aspects, this is performed by co-partitioning the individual biological particle or groups of biological particles with the unique identifiers, such as described above (with reference to FIG. 3 or 4).

In various cases, additional beads can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of beads from each source, while ensuring a given pairing or combination of such beads into a partition with a given number of biological particles (e.g., one biological particle and one bead per partition).

### Wells:

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). In some embodiments, a well of a fluidic device is fluidically connected to another well of the fluidic device. Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In various instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In various instances, a microwell array or plate comprises a single variety of microwells. In various instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a droplet, bead, or other reagent. In various cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

A well may comprise free reagents and/or reagents encapsulated in, or otherwise coupled to or associated with, beads, beads, or droplets.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In various cases, a well comprises a bead, or droplet that comprises a set of reagents that has a similar attribute (e.g., a set of enzymes, a set of minerals, a set of oligonucleotides, a mixture of different barcode molecules, a mixture of identical barcode molecules). In other cases, a bead or droplet comprises a heterogeneous mixture of reagents. In various cases, the heterogeneous mixture of reagents can comprise all components necessary to perform a reaction. In various cases, such mixture can comprise all components necessary to perform a reaction, except for 1, 2, 3, 4, 5, or more components necessary to perform a reaction. In various cases, such additional components are contained within, or otherwise coupled to, a different droplet or bead, or within a solution within a partition (e.g., microwell) of the system.

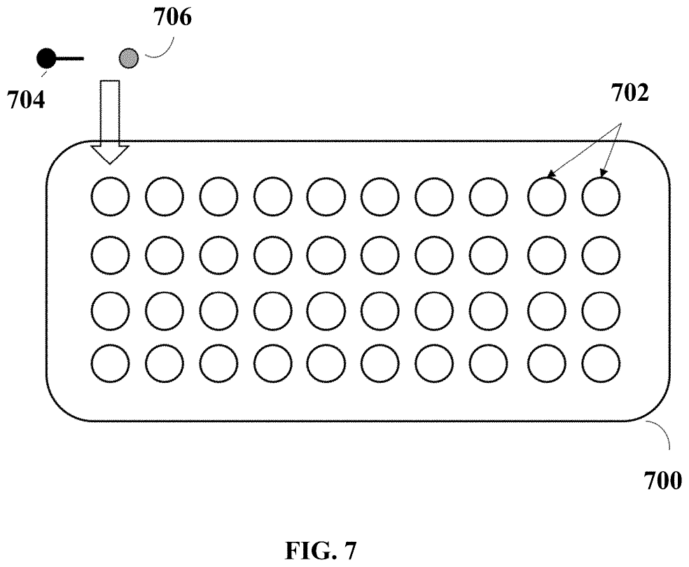

FIG. 7 schematically illustrates an example of a microwell array. The array can be contained within a substrate 700. The substrate 700 comprises a plurality of wells 702. The wells 702 may be of any size or shape, and the spacing between the wells, the number of wells per substrate, as well as the density of the wells on the substrate 700 can be modified, depending on the particular application. In one such example application, a sample molecule 706, which may comprise a cell or cellular components (e.g., nucleic acid molecules) is co-partitioned with a bead 704, which may comprise a nucleic acid barcode molecule coupled thereto. The wells 702 may be loaded using gravity or other loading technique (e.g., centrifugation, liquid handler, acoustic loading, optoelectronic, etc.). In various instances, at least one of the wells 702 contains a single sample molecule 706 (e.g., cell) and a single bead 704.

Reagents may be loaded into a well either sequentially or concurrently. In various cases, reagents are introduced to the device either before or after a particular operation. In various cases, reagents (which may be provided, in certain instances, in droplets, or beads) are introduced sequentially such that different reactions or operations occur at different steps. The reagents (or droplets, or beads) may also be loaded at operations interspersed with a reaction or operation step. For example, beads (or droplets) comprising reagents for fragmenting polynucleotides (e.g., restriction enzymes) and/or other enzymes (e.g., transposases, ligases, polymerases, etc.) may be loaded into the well or plurality of wells, followed by loading of droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead, or droplet. These beads, or droplets may be loaded into a partition (e.g., a microwell) before, after, or concurrently with the loading of a cell, such that each cell is contacted with a different bead, or droplet. This technique may be used to attach a unique nucleic acid barcode molecule to nucleic acid molecules obtained from each cell. Alternatively, or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in various instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in various instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In various cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or bead may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In various instances, the wells can comprise nucleic acid barcode molecules attached thereto. The nucleic acid barcode molecules may be attached to a surface of the well (e.g., a wall of the well). The nucleic acid barcode molecules may be attached to a droplet or bead that has been partitioned into the well. The nucleic acid barcode molecule (e.g., a partition barcode sequence) of one well may differ from the nucleic acid barcode molecule of another well, which can permit identification of the contents contained with a single partition or well. In various cases, the nucleic acid barcode molecule can comprise a spatial barcode sequence that can identify a spatial coordinate of a well, such as within the well array or well plate. In various cases, the nucleic acid barcode molecule can comprise a unique molecular identifier for individual molecule identification. In various instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In various instances, the nucleic acid barcode molecules may be releasable from the microwell. In some instances, the nucleic acid barcode molecules may be releasable from the bead or droplet. For instance, the nucleic acid barcode molecules may comprise a chemical cross-linker which may be cleaved upon application of a stimulus (e.g., photo-, magnetic, chemical, biological, stimulus). The nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In some instances nucleic acid barcode molecules attached to a bead in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In various instances, imaging may be used to characterize live cells in the wells, including, but not limited to: dynamic live-cell tracking, cell-cell interactions (when two or more cells are co-partitioned), cell proliferation, etc. Alternatively, or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

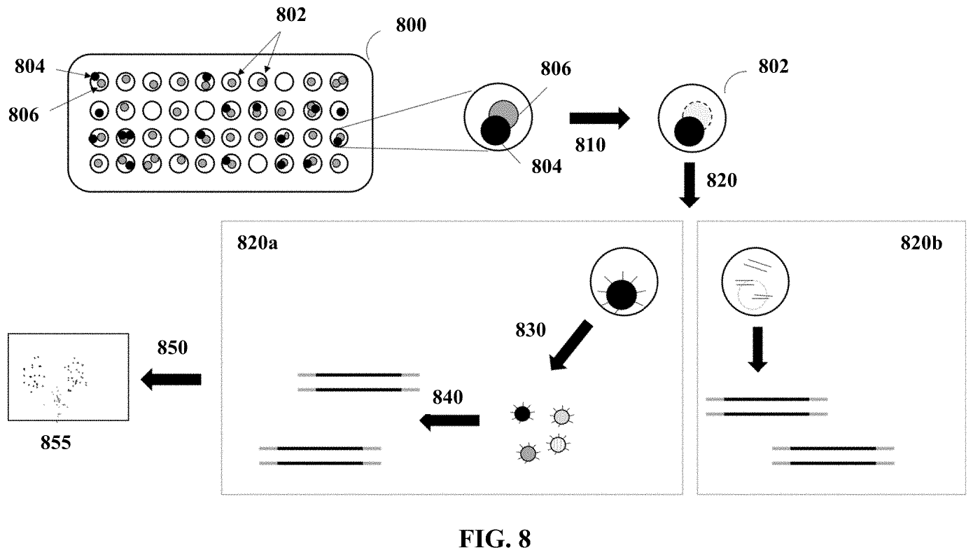

FIG. 8 schematically shows an example workflow for processing nucleic acid molecules within a sample. A substrate 800 comprising a plurality of microwells 802 may be provided. A sample 806 which may comprise a cell, cell bead, cellular components or analytes (e.g., proteins and/or nucleic acid molecules) can be co-partitioned, in a plurality of microwells 802, with a plurality of beads 804 comprising nucleic acid barcode molecules. During process 810, the sample 806 may be processed within the partition. For instance, in the case of live cells, the cell may be subjected to conditions sufficient to lyse the cells and release the analytes contained therein. In process 820, the bead 804 may be further processed. By way of example, processes 820a and 820b schematically illustrate different workflows, depending on the properties of the bead 804.

In 820a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 830, the beads 804 from multiple wells 802 may be collected and pooled. Further processing may be performed in process 840. For example, one or more nucleic acid reactions may be performed, such as reverse transcription, nucleic acid extension, amplification, ligation, transposition, etc. In various instances, adapter sequences are ligated to the nucleic acid molecules, or derivatives thereof, as described elsewhere herein. For instance, sequencing primer sequences may be appended to each end of the nucleic acid molecule. In process 850, further characterization, such as sequencing may be performed to generate sequencing reads. The sequencing reads may yield information on individual cells or populations of cells, which may be represented visually or graphically, e.g., in a plot 855.

In 820b, the bead comprises nucleic acid barcode molecules that are releasably attached thereto, as described below. The bead may degrade or otherwise release the nucleic acid barcode molecules into the well 802; the nucleic acid barcode molecules may then be used to barcode nucleic acid molecules within the well 802. Further processing may be performed either inside the partition or outside the partition. For example, one or more nucleic acid reactions may be performed, such as reverse transcription, nucleic acid extension, amplification, ligation, transposition, etc. In various instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 855.

### Targeted Enrichment

The methods provided herein may comprise the use of a targeting process to, for example, enrich selected nucleic acid molecules within a sample.

An exemplary target enrichment method may comprise providing a plurality of barcoded nucleic acid molecules and hybridizing barcoded nucleic acid molecules comprising targeted regions of interest to oligonucleotide probes (“baits”) which are complementary to the targeted regions of interest (or to regions near or adjacent to the targeted regions of interest). Baits may be attached to a capture molecule, including without limitation a biotin molecule. The capture molecule (e.g., biotin) can be used to selectively pull down the targeted regions of interest (for example, with magnetic streptavidin beads) to thereby enrich the resultant population of barcoded nucleic acid molecules for those containing the targeted regions of interest.

### Multiplexing:

The present disclosure provides methods and systems for multiplexing, and otherwise increasing throughput in, analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cell features may be used to characterize biological particles and/or cell features. In various instances, cell features include cell surface features. In various instances, a cell feature is a cell surface molecule. Exemplary cell surface molecules are disclosed herein. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In various instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, an aptamer-payload complex disclosed herein, an internalization complex disclosed herein, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, the disclosures of which are incorporated by reference in their entireties for all purposes.

In a particular example, a library of potential cell feature labelling agents (e.g., a library of aptamer-payload complexes, a library of internalization complexes) may be provided, where the respective cell feature labelling agents are associated with nucleic acid reporter molecules (e.g., comprising an aptamer barcode sequence), such that a different reporter oligonucleotide sequence (e.g., a different aptamer barcode sequence) is associated with each labelling agent (e.g., each aptamer-payload complex or each internalization complex) capable of binding to a specific cell feature. In various aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

Labelling agents capable of binding to or otherwise coupling to one or more biological particles may be used to characterize a biological particle as belonging to a particular set of biological particles. For example, labeling agents may be used to label a sample of cells or a group of cells. In this way, a group of cells may be labeled as different from another group of cells. In an example, a first group of cells may originate from a first sample and a second group of cells may originate from a second sample. Labelling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling a biological particle may comprise subjecting the antibody-linked barcode molecule or the epitope binding fragment-linked barcode molecule to conditions suitable for binding the antibody to a molecule present on a surface of the biological particle. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant may be less than about 10 μM.

In another example, a reporter oligonucleotide may be coupled to a cell-penetrating peptide (CPP), and labeling cells may comprise delivering the CPP coupled reporter oligonucleotide into a biological particle. Labeling biological particles may comprise delivering the CPP conjugated oligonucleotide into a cell and/or cell bead by the cell-penetrating peptide. A cell-penetrating peptide that can be used in the methods provided herein can comprise at least one non-functional cysteine residue, which may be either free or derivatized to form a disulfide link with an oligonucleotide that has been modified for such linkage. Non-limiting examples of cell-penetrating peptides that can be used in embodiments herein include penetratin, transportan, plsl, TAT (48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The cell-penetrating peptide may be an arginine-rich peptide transporter. The cell-penetrating peptide may be Penetratin or the Tat peptide.

In another example, a reporter oligonucleotide may be coupled to a fluorophore or dye, and labeling cells may comprise subjecting the fluorophore-linked barcode molecule to conditions suitable for binding the fluorophore to the surface of the biological particle. In various instances, fluorophores can interact strongly with lipid bilayers and labeling biological particles may comprise subjecting the fluorophore-linked barcode molecule to conditions such that the fluorophore binds to or is inserted into a membrane of the biological particle. In various cases, the fluorophore is a water-soluble, organic fluorophore. In various instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is hereby incorporated by reference in its entirety for all purposes, for a description of organic fluorophores.

A reporter oligonucleotide may be coupled to a lipophilic molecule, and labeling biological particles may comprise delivering the nucleic acid barcode molecule to a membrane of the biological particle or a nuclear membrane by the lipophilic molecule. Lipophilic molecules can associate with and/or insert into lipid membranes such as cell membranes and nuclear membranes. In various cases, the insertion can be reversible. In various cases, the association between the lipophilic molecule and biological particle may be such that the biological particle retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells may be incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, the disclosure of which is hereby incorporated by reference in its entirety for all purposes.

As described elsewhere herein, libraries of labelling agents may be associated with a particular cell feature as well as be used to identify analytes as originating from a particular biological particle, population, or sample. The biological particles may be incubated with a plurality of libraries and a given biological particle may comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In various instances, these reporter oligonucleotides may comprise nucleic acid barcode sequences that permit identification of the labelling agent to which the reporter oligonucleotide is coupled. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment), e.g., via a linker, using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, the disclosure of which is incorporated by reference herein in its entirety for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In various embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In various instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In various cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In various cases, a label is conjugated to an oligonucleotide that is complementary to a sequence of the reporter oligonucleotide, and the oligonucleotide may be allowed to hybridize to the reporter oligonucleotide.

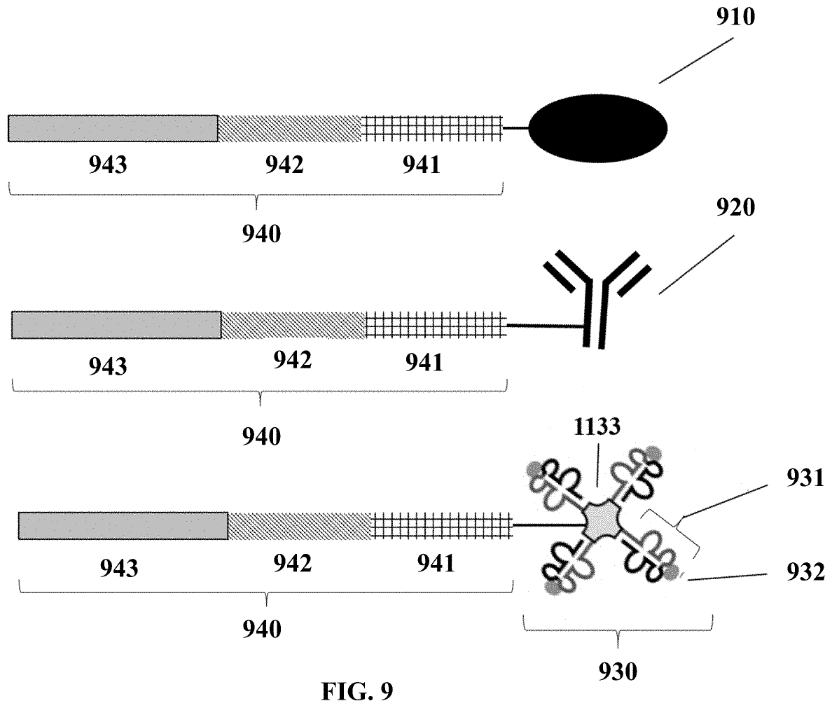

FIG. 9 describes exemplary labelling agents (910, 920, 930) comprising reporter oligonucleotides (940) attached thereto. Labelling agent 910 (e.g., any of the labelling agents described herein) is attached (either directly, e.g., covalently attached, or indirectly) to reporter oligonucleotide 940. Reporter oligonucleotide 940 may comprise barcode sequence 942 that identifies labelling agent 910. Reporter oligonucleotide 940 may also comprise one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer binding sequence (such as an R1, R2, or partial R1 or R2 sequence).

Referring to FIG. 9, in various instances, reporter oligonucleotide 940 conjugated to a labelling agent (e.g., 910, 920, 930) comprises a functional sequence 941 (e.g., a primer sequence), a barcode sequence that identifies the labelling agent (e.g., 910, 920, 930), and functional sequence 943. Functional sequence 943 can be a reporter capture handle sequence configured to hybridize to a complementary sequence, such as a complementary sequence present on a nucleic acid barcode molecule 990 (not shown), such as those described elsewhere herein. In various instances, nucleic acid barcode molecule 990 is attached to a support (e.g., a bead, such as a gel bead), such as those described elsewhere herein. For example, nucleic acid barcode molecule 990 may be attached to the support via a releasable linkage (e.g., comprising a labile bond), such as those described elsewhere herein. In various instances, reporter oligonucleotide 940 comprises one or more additional functional sequences, such as those described above.

In various instances, the labelling agent 910 is a protein or polypeptide (e.g., an antigen or prospective antigen) comprising reporter oligonucleotide 940. Reporter oligonucleotide 940 comprises barcode sequence 942 that identifies polypeptide 910 and can be used to infer the presence of an analyte, e.g., a binding partner of polypeptide 910 (i.e., a molecule or compound to which polypeptide 910 can bind). In various instances, the labelling agent 910 is a lipophilic moiety (e.g., cholesterol) comprising reporter oligonucleotide 940, where the lipophilic moiety is selected such that labelling agent 910 integrates into a membrane of a cell or nucleus. Reporter oligonucleotide 940 comprises barcode sequence 942 that identifies lipophilic moiety 910 which in various instances is used to tag cells (e.g., groups of cells, cell samples, etc.) and may be used for multiplex analyses as described elsewhere herein. In various instances, the labelling agent is an antibody 920 (or an epitope binding fragment thereof) comprising reporter oligonucleotide 940. Reporter oligonucleotide 940 comprises barcode sequence 942 that identifies antibody 920 and can be used to infer the presence of, e.g., a target of antibody 920 (i.e., a molecule or compound to which antibody 920 binds). In other embodiments, labelling agent 930 comprises an MHC molecule 931 comprising peptide 932 and reporter oligonucleotide 940 that identifies peptide 932. In various instances, the MEW molecule is coupled to a support 933. In various instances, support 933 may be or comprise a polypeptide, such as streptavidin, avidin, neutravidin, or a polysaccharide, such as dextran. In some embodiments, support 933 further comprises a detectable label, e.g., a detectable label described herein, e.g., fluorescent label. In various instances, reporter oligonucleotide 940 may be directly or indirectly coupled to MHC labelling agent 930 in any suitable manner. For example, reporter oligonucleotide 940 may be coupled to MHC molecule 931, support 933, or peptide 932. In various embodiments, labelling agent 930 comprises a plurality of MHC molecules described herein, (e.g. is an MEW multimer, which may be coupled to a support (e.g., 933)). In some embodiments, reporter oligonucleotide 940 and an antigen (e.g., protein, polypeptide) are attached to polypeptide or polysaccharide of support 933. In some embodiments, reporter oligonucleotide 940 and an antigen (e.g., protein, polypeptide) are attached to the detectable label of support 933. There are many possible configurations of Class I and/or Class II MHC multimers that can be utilized with the compositions, methods, and systems disclosed herein, e.g., MHC tetramers, MHC pentamers (MEW assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969, the disclosures of which are herein incorporated by reference in their entireties for all purposes.

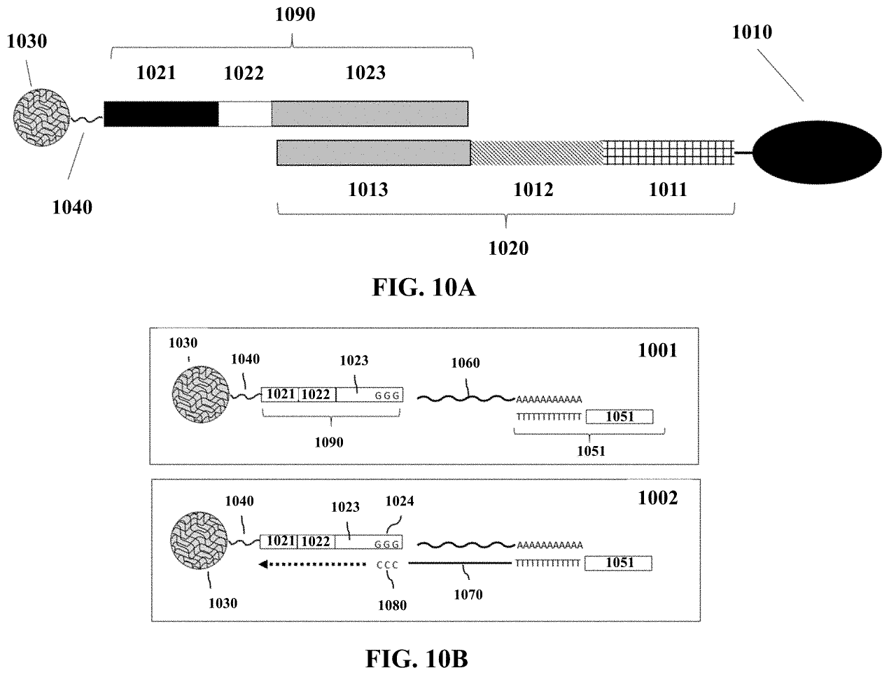

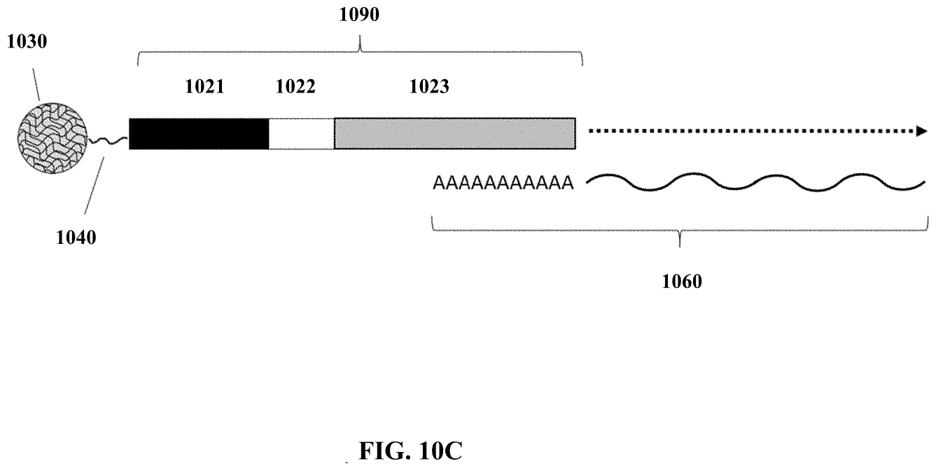

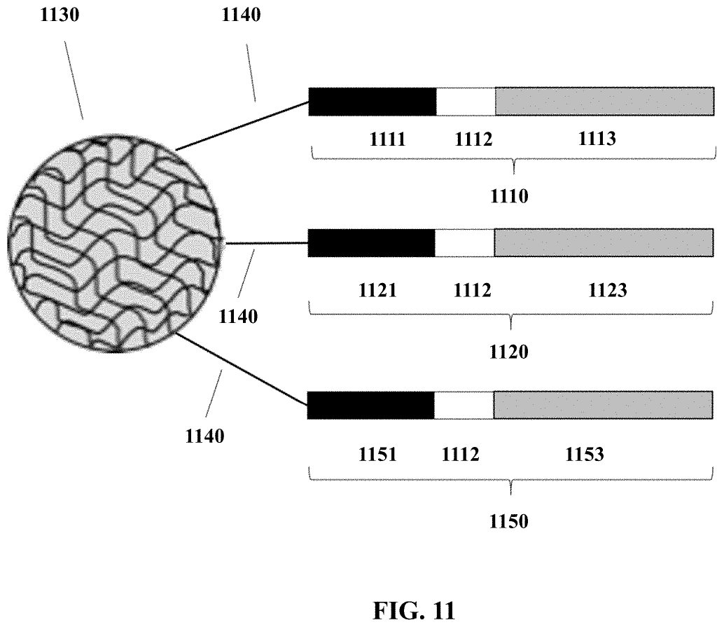

FIG. 11 illustrates another example of a barcode carrying bead. In various embodiments, analysis of multiple analytes (e.g., RNA and one or more analytes using labelling agents described herein) may comprise nucleic acid barcode molecules as generally depicted in FIG. 11. In various embodiments, nucleic acid barcode molecules 1110 and 1120 are attached to support 1130 via a releasable linkage 1140 (e.g., comprising a labile bond) as described elsewhere herein. Nucleic acid barcode molecule 1110 may comprise adapter sequence 1111, barcode sequence 1112 and capture sequence 1113. Nucleic acid barcode molecule 1120 may comprise adapter sequence 1121, barcode sequence 1112, and capture sequence 1123, wherein capture sequence 1123 comprises a different sequence than capture sequence 1113. In various instances, adapter 1111 and adapter 1121 comprise the same sequence. In various instances, adapter 1111 and adapter 1121 comprise different sequences. Although support 1130 is shown comprising nucleic acid barcode molecules 1110 and 1120, any suitable number of barcode molecules comprising common barcode sequence 1112 are contemplated herein. For example, in various embodiments, support 1130 further comprises nucleic acid barcode molecule 1150. Nucleic acid barcode molecule 1150 may comprise adapter sequence 1151, barcode sequence 1112 and capture sequence 1153, wherein capture sequence 1153 comprises a different sequence than capture sequence 1113 and 1123. In various instances, nucleic acid barcode molecules (e.g., 1110, 1120, 1150) comprise one or more additional functional sequences, such as a UMI or other sequences described herein. The nucleic acid barcode molecules 1110, 1120 or 1150 may interact with analytes as described elsewhere herein, for example, as depicted in FIGS. 10A-C.

Referring to FIG. 10A, in an instance where cells are labelled with labeling agents, capture sequence 1023 may be complementary to an adapter sequence of a reporter oligonucleotide. Cells may be contacted with one or more reporter oligonucleotide 1020 conjugated labelling agents 1010 (e.g., polypeptide, antibody, or others described elsewhere herein). In various cases, the cells may be further processed prior to barcoding. For example, such processing steps may include one or more washing and/or cell sorting steps. In various instances, a cell that is bound to labelling agent 1010 which is conjugated to oligonucleotide 1020 and support 1030 (e.g., a bead, such as a gel bead) comprising nucleic acid barcode molecule 1090 is partitioned into a partition amongst a plurality of partitions (e.g., a droplet of a droplet emulsion or a well of a microwell array). In various instances, the partition comprises at most a single cell bound to labelling agent 1010. In various instances, reporter oligonucleotide 1020 conjugated to labelling agent 1010 (e.g., polypeptide, an antibody, pMHC molecule such as an MHC multimer, etc.) comprises a first adapter sequence 1011 (e.g., a primer sequence), a barcode sequence 1012 that identifies the labelling agent 1010 (e.g., the polypeptide, antibody, or peptide of a pMHC molecule or complex), and an capture handle sequence 1013. Capture handle sequence 1013 may be configured to hybridize to a complementary sequence, such as a capture sequence 1023 present on a nucleic acid barcode molecule 1090. In various instances, oligonucleotide 1020 comprises one or more additional functional sequences, such as those described elsewhere herein.

Barcoded nucleic may be generated (e.g., via a nucleic acid reaction, such as nucleic acid extension or ligation) from the constructs described in FIGS. 10A-C. For example, capture handle sequence 1013 may then be hybridized to complementary sequence, such as capture sequence 1023 to generate (e.g., via a nucleic acid reaction, such as nucleic acid extension or ligation) a barcoded nucleic acid molecule comprising cell (e.g., partition specific) barcode sequence 1022 (or a reverse complement thereof) and reporter barcode sequence 1012 (or a reverse complement thereof). In various embodiments, the nucleic acid barcode molecule 1090 (e.g., partition-specific barcode molecule) further includes a UMI (not shown). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, the disclosure of which is hereby incorporated by reference in its entirety herein for all purposes. Barcoded nucleic acid molecules, or derivatives generated therefrom, can then be sequenced on a suitable sequencing platform.

In various instances, analysis of multiple analytes (e.g., nucleic acids and one or more analytes using labelling agents described herein) may be performed. For example, the workflow may comprise a workflow as generally depicted in any of FIGS. 10A-C, or a combination of workflows for an individual analyte, as described elsewhere herein. For example, by using a combination of the workflows as generally depicted in FIGS. 10A-C, multiple analytes can be analyzed.

In various instances, analysis of an analyte (e.g. a nucleic acid, a polypeptide, a carbohydrate, a lipid, etc.) comprises a workflow as generally depicted in FIG. 10A. A nucleic acid barcode molecule 1090 may be co-partitioned with the one or more analytes. In various instances, nucleic acid barcode molecule 1090 is attached to a support 1030 (e.g., a bead, such as a gel bead), such as those described elsewhere herein. For example, nucleic acid barcode molecule 1090 may be attached to support 1030 via a releasable linkage 1040 (e.g., comprising a labile bond), such as those described elsewhere herein. Nucleic acid barcode molecule 1090 may comprise a functional sequence 1021 and optionally comprise other additional sequences, for example, a barcode sequence 1022 (e.g., common barcode, partition-specific barcode, or other functional sequences described elsewhere herein), and/or a UMI sequence (not shown). The nucleic acid barcode molecule 1090 may comprise a capture sequence 1023 that may be complementary to another nucleic acid sequence, such that it may hybridize to a particular sequence, e.g., capture handle sequence 1013.

For example, capture sequence 1023 may comprise a poly-T sequence and may be used to hybridize to mRNA. Referring to FIG. 10C, in various embodiments, nucleic acid barcode molecule 1090 comprises capture sequence 1023 complementary to a sequence of RNA molecule 1060 from a cell. In various instances, capture sequence 1023 comprises a sequence specific for an RNA molecule. Capture sequence 1023 may comprise a known or targeted sequence or a random sequence. In various instances, a nucleic acid extension reaction may be performed, thereby generating a barcoded nucleic acid product comprising capture sequence 1023, the functional sequence 1021, barcode sequence 1022, any other functional sequence, and a sequence corresponding to the RNA molecule 1060.

In another example, capture sequence 1023 may be complementary to an overhang sequence or an adapter sequence that has been appended to an analyte. For example, referring to FIG. 10B, panel 1001, in various embodiments, primer 1050 comprises a sequence complementary to a sequence of nucleic acid molecule 1060 (such as an RNA encoding for a BCR sequence) from a biological particle. In various instances, primer 1050 comprises one or more sequences 1051 that are not complementary to RNA molecule 1060. Sequence 1051 may be a functional sequence as described elsewhere herein, for example, an adapter sequence, a sequencing primer sequence, or a sequence the facilitates coupling to a flow cell of a sequencer. In various instances, primer 1050 comprises a poly-T sequence. In various instances, primer 1050 comprises a sequence complementary to a target sequence in an RNA molecule. In various instances, primer 1050 comprises a sequence complementary to a region of an immune molecule, such as the constant region of a TCR or BCR sequence. Primer 1050 is hybridized to nucleic acid molecule 1060 and complementary molecule 1070 is generated (see Panel 1202). For example, complementary molecule 1070 may be cDNA generated in a reverse transcription reaction. In various instances, an additional sequence may be appended to complementary molecule 1070. For example, the reverse transcriptase enzyme may be selected such that several non-templated bases 1080 (e.g., a poly-C sequence) are appended to the cDNA. In another example, a terminal transferase may also be used to append the additional sequence. Nucleic acid barcode molecule 1090 comprises a sequence 1024 complementary to the non-templated bases, and the reverse transcriptase performs a template switching reaction onto nucleic acid barcode molecule 1090 to generate a barcoded nucleic acid molecule comprising cell (e.g., partition specific) barcode sequence 1022 (or a reverse complement thereof) and a sequence of complementary molecule 1070 (or a portion thereof). In various instances, sequence 1023 comprises a sequence complementary to a region of an immune molecule, such as the constant region of a TCR or BCR sequence. Sequence 1023 is hybridized to nucleic acid molecule 1060 and a complementary molecule 1070 is generated. For example, complementary molecule 1070 may be generated in a reverse transcription reaction generating a barcoded nucleic acid molecule comprising cell (e.g., partition specific) barcode sequence 1022 (or a reverse complement thereof) and a sequence of complementary molecule 1070 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969, the disclosures of which are herein incorporated by reference in their entireties for all purposes.

### Combinatorial Barcoding:

In some instances, barcoding of a nucleic acid molecule may be done using a combinatorial approach. In such instances, one or more nucleic acid molecules (which may be comprised in a biological particle, e.g., a cell, e.g., a fixed cell, organelle, nucleus, or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The barcoded nucleic acid molecules may thus comprise two barcode sequences (e.g., from the first nucleic acid barcode molecules and the second nucleic acid barcode molecules).

Additional barcode sequences may be attached to the barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 5, 6, 7, 8, 9, 10 or more operations of splitting (e.g., partitioning) and/or pooling (e.g., from the partitions). Additional examples of combinatorial barcoding may also be found in International Patent Publication Nos. WO2019/165318, each of which is herein entirely incorporated by reference for all purposes.

Beneficially, the combinatorial barcode approach may be useful for generating greater barcode diversity, and synthesizing unique barcode sequences on nucleic acid molecules derived from a cell or partition. For example, combinatorial barcoding comprising three operations, each with 100 partitions, may yield up to 106 unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g., a sample or target nucleic acid molecule) in successive operations within a partition (e.g., droplet or well) to generate a barcoded nucleic acid molecule. A second unique nucleic acid molecule comprising one or more nucleic acid bases may be attached to the barcoded nucleic acid molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in PCT/US2018/067356 and U.S. Pat. Pub. No. 2019/0330694, which are hereby incorporated by reference in its entirety.

### Alternative Systems and Methods for Partitioning

In some instances wherein a partition is a volume wherein diffusion of contents beyond the volume is inhibited, the partition contains a diffusion resistant material. Such partition may also be referred to herein as a diffusion resistant partition. The diffusion resistant material may have an increased viscosity. The diffusion resistant material may be or comprise a matrix, e.g., a polymeric matrix, or a gel. Suitable polymers or gels are disclosed herein. The matrix can be a porous matrix capable of entraining and/or retaining materials within its matrix. In some embodiments, a diffusion resistant partition comprises a single biological particle and a single bead, the single bead comprising a plurality of nucleic acid barcode molecules comprising a partition specific barcode sequence. In some embodiments the partition specific barcode sequence is unique to the diffusion resistant partition. In some embodiments, partitioning comprises contacting a plurality of biological particles with a plurality of beads in a diffusion resistant material to provide a diffusion resistant partition comprising a single biological particle and a single bead. In some embodiments, partitioning comprises contacting a plurality of biological particles with a plurality of beads in a liquid comprising a polymeric precursor material that may be capable of being formed into a gel or other solid or semi-solid matrix, and subjecting the liquid to conditions sufficient to polymerize or gel the precursors, e.g., as described herein. In some embodiments, the biological particle may be lysed or permeabilized in the diffusion resistant partition. In some embodiments, a nucleic acid analyte of the biological particle (which may include a reporter oligonucleotide associated with a labelling agent disclosed herein) may be coupled with a nucleic acid barcode molecule in the diffusion resistant partition. In some cases, further processing, e.g., generation of barcoded nucleic acid molecules, may be performed in the diffusion resistant partition or in bulk. For example, nucleic acid analytes, once coupled to nucleic acid barcode molecules in partitions, may be pooled and then subjected to further processing in bulk (e.g, extension, reverse transcription, or other processing) to generate barcoded nucleic acid molecules. For other example, nucleic acid analytes, one coupled to nucleic acid barcode molecules in diffusion resistant partitions, may be subjected to further processing in the diffusion resistant partitions to generate barcoded nucleic acid molecules

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification can have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences can be varied and still remain within the spirit and scope of the various embodiments.

1. A method for internalization of a plurality of payloads into a cell, the method comprising:
(a) contacting a cell with an internalization complex, the internalization complex comprising (i) a first aptamer comprising a 3′ end, a 5′ end, a cell surface binding domain, and a hybridization sequence, (ii) a second aptamer comprising a 3′ end, a 5′ end, first hybridization sequence, a second hybridization sequence, and a payload binding domain, (iii) a third aptamer comprising a 3′ end, a 5′ end, a payload binding domain, and a hybridization sequence,
wherein the hybridization sequence of the first aptamer is configured to hybridize to the first hybridization sequence of the second aptamer, and
wherein the hybridization sequence of the third aptamer is configured to hybridize to the second hybridization sequence of the second aptamer;
(b) hybridizing the hybridization sequence of the first aptamer to the first hybridization sequence of the second aptamer and hybridizing the hybridization sequence of the third aptamer to the second hybridization sequence of the second aptamer to create an internalization complex; and
(c) contacting the payload binding domain of the second aptamer to a first payload and contacting the payload binding domain of the third aptamer to a second payload.

2. A method for internalization of a plurality of payloads into a cell, the method comprising:
(a) contacting a cell with an internalization complex, the internalization complex comprising (i) a first aptamer comprising a 3′ end, a 5′ end, a cell surface binding domain, a first hybridization sequence, and a second hybridization sequence, (ii) a second aptamer comprising a 3′ end, a 5′ end, a hybridization sequence, and a payload binding domain, and (iii) a third aptamer comprising a 3′ end, a 5′ end, a hybridization sequence, and a payload binding domain,
wherein the first hybridization sequence of the first aptamer is configured to hybridize to the hybridization sequence of the second aptamer, and
wherein the second hybridization sequence of the first aptamer is configured to hybridize to the hybridization sequence of the third aptamer;
(b) hybridizing the first hybridization sequence of the first aptamer to the hybridization sequence of the second aptamer and hybridizing the second hybridization sequence of the first aptamer to the hybridization sequence of the third aptamer to create an internalization complex; and
(c) binding the payload binding domain of the second aptamer to a first payload and binding the payload binding domain of the third aptamer to a second payload.

3. The method of claim 1 or 2, further comprising the steps of:
(d) contacting a cell with the internalization complex;
(e) binding the cell surface binding domain to a cell surface molecule; and
(f) internalizing the internalization complex.

4. The method of claim 1 or 2, further comprising the step of activating a detectable label upon:
binding one of the payload binding domains to one of the payloads; or
binding the cell surface binding domain to the cell surface molecule.

5. The method of claim 3, further comprising the step of activating a detectable label upon internalization of the internalization complex.

6. The method of claim 4, wherein the first, the second or the third aptamer comprises the detectable label.

7. The method of claim 5, wherein the first, the second or the third aptamer comprises the detectable label.

8. A method for internalization of a payload into a cell, the method comprising:
(a) contacting a cell with an internalization complex, the internalization complex comprising (i) a first aptamer comprising a 3′ end, a 5′ end, a cell surface binding domain, and a first hybridization sequence, (ii) a second aptamer comprising a 3′ end, a 5′ end, a payload binding domain, and a second hybridization sequence, and (iii) a payload, wherein the first hybridization sequence is hybridized to the second hybridization sequence, and wherein the payload is bound to the payload binding domain of the second aptamer;
(b) binding the cell surface binding domain to a cell surface molecule of the cell; and
(c) internalizing the internalization complex into a cell.

9. The method according to claim 8, further comprising the step of activating a detectable label upon:
binding the payload binding domain to the payload; or
binding the cell surface binding domain to the cell surface molecule; or
internalization of the internalization complex.

10. The method according to claim 9, wherein the first, or the second, aptamer comprises the detectable label.

11. The method of claim 4, further comprising the step of emitting a light signal from the detectable label using a fluorescent molecule.

12. The method of claim 5, further comprising the step of emitting a light signal from the detectable label using a fluorescent molecule.

13. The method according to claim 11, further comprising the step of quenching the emitted light signal from the detectable label by interacting a quenching agent with the fluorescent molecule.

14. The method according to claim 12, further comprising the step of quenching the emitted light signal from the detectable label by interacting a quenching agent with the fluorescent molecule.

15. The method of claim 3, further comprising the step of confirming internalization of the internalization complex.

16. The method of claim 15, wherein at least one of the first aptamer, the second aptamer, and the third aptamer comprises an aptamer barcode sequence.

17. The method according to claim 16, further comprising the step of sequencing the at least one of the aptamer barcode sequence.

18. The method according to any one of claims claim 1 or 2, further comprising the step of confirming correct aptamer pairing, and
optionally, wherein the confirming correct aptamer pairing comprises a fluorescence in situ hybridization method.

19. The method according to claim 3, further comprising, after the internalizing:
partitioning the cell into a partition with a plurality of nucleic acid barcode molecules, wherein a nucleic acid barcode molecule of the plurality of nucleic acid barcode molecules comprises a partition-specific barcode sequence and a capture sequence that is complementary to at least a portion of at least one of the first aptamer, the second aptamer, or the third aptamer one of the aptamers; and
hybridizing the capture sequence of the nucleic acid barcode molecule to the at least a portion of the one of the aptamers; and
using the nucleic acid barcode molecule and the one of the aptamers to generate a barcoded product comprising the partition-specific barcode sequence or a complement thereof and the aptamer barcode sequence or a complement thereof.

20. The method of claim 19, wherein the partitioning the cell into the partition comprises partitioning the cell and a bead into the partition, wherein the bead comprises the plurality of nucleic acid barcode molecules;
optionally, wherein the nucleic acid barcode molecule of the plurality of nucleic acid barcode molecules further comprises a unique molecular identifier (UMI).

21. The method of claim 19, further comprising:
determining the sequence of the barcoded product or a portion thereof; and
confirming internalization of the internalization complex in the cell if the sequence of the barcoded product or portion thereof contains (i) the partition-specific barcode sequence or complement thereof and (ii) the aptamer barcode sequence or complement thereof.

22. A composition for internalizing a payload into a cell, the composition comprising:
(I) (a) a first aptamer comprising:
(i) a 3′ end;
(ii) a 5′ end;
(iii) a cell surface binding domain; and
(iv) a hybridization sequence; and
(b) a second aptamer comprising:
(i) a 3′ end;
(ii) a 5′ end;
(iii) a payload binding domain; and
(iv) a hybridization sequence;
wherein the hybridization sequence of the first aptamer is configured to hybridize to the hybridization sequence of the second aptamer; or
(II)(a) a first aptamer comprising:
(i) a 3′ end;
(ii) a 5′ end;
(iii) a cell surface binding domain; and
(iv) a hybridization sequence;
(b) a second aptamer comprising:
(i) a 3′ end;
(ii) a 5′ end;
(iii) a first hybridization sequence;
(iv) a second hybridization sequence; and
(v) a payload binding domain;
(c) a third aptamer comprising:
(i) a 3′ end;
(ii) a 5′ end;
(iii) a payload binding domain; and
(iv) a hybridization sequence,
wherein the hybridization sequence of the first aptamer is configured to hybridize to the first hybridization sequence of the second aptamer, and
wherein the hybridization sequence of the third aptamer is configured to hybridize to the second hybridization sequence of the second aptamer; or
(III) (a) a first aptamer comprising:
a 3′ end;
(ii) a 5′ end;
(iii) a cell surface binding domain;
(iv) a first hybridization sequence; and
(v) a second hybridization sequence;
(b) a second aptamer comprising:
(i) a 3′ end;
(ii) a 5′ end;
(iii) a hybridization sequence; and
(iv) a payload binding domain;
(c) a third aptamer comprising:
(i) a 3′ end;
(ii) a 5′ end;
(iii) a hybridization sequence; and
(iv) a payload binding domain,
wherein the first hybridization sequence is configured to hybridize to the hybridization sequence of the second aptamer, and
wherein the second hybridization sequence is configured to hybridize to the hybridization sequence of the third aptamer.

23. The composition of claim 22(I), wherein the first or the second aptamer further comprises a detectable label,
optionally wherein the detectable label comprises a fluorescent molecule,
optionally wherein the detectable label comprising the fluorescent molecule further comprises a quenching agent configured to interact with the fluorescent molecule and decrease fluorescence.

24. The composition of claim 22(II) or 22(III), wherein the first, the second or the third aptamer further comprises a detectable label,
optionally wherein the detectable label comprises a fluorescent molecule, and
optionally wherein the detectable label comprising the fluorescent molecule further comprises a quenching agent configured to interact with the fluorescent molecule and decrease fluorescence.

25. The composition according to any one of claim 23 or 24, wherein the detectable label is activated upon: internalization of at least one of the aptamers into a cell, binding of the second aptamer to a payload, or binding of the first aptamer to a cell surface molecule.

26. The composition of claim 22, wherein at least one of the aptamers comprises an aptamer barcode sequence.

27. The composition of claim 22, further comprising a cell surface molecule bound to the cell surface binding domain,
optionally wherein the cell surface molecule is part of an internalization complex.

28. The composition of claim 22, wherein the payload binding domain further comprises a payload handle, and
optionally, wherein the payload handle comprises a protein recognition sequence, a biotinylated structure, or a poly-A tail capture sequence.

29. The composition of claim 22, further comprising a payload bound to the payload binding domain, and
optionally, wherein the payload comprises a Cas molecule, a gRNA molecule, an RNP complex comprising a Cas molecule associated with a gRNA molecule, an mRNA molecule, an oligonucleotide a protein or an RNAi molecule.

30. The composition of claim 22, further comprising a bead comprising an oligonucleotide, wherein the oligonucleotide comprises:
a nucleic acid barcode molecule comprising a bead specific barcode; and
a capture sequence, wherein the capture sequence is complementary to at least a portion of at least one of the aptamers.

31. A kit for internalizing a payload into a cell, comprising:
(i) the first and the second aptamer as recited in the composition of claim 22(I),
a payload, and
instructions for use;
or
(ii) the first, the second and the third aptamer as recited in the composition of claim 22(II) or claim 22(III),
a plurality of payloads, and
instructions for use.
